SEQUENCE LISTING (262 sequences)

SEQ ID NO: 1 - protein, 25 aa, Artificial Sequence [sequence omitted]

SEQ ID NO: 2 - protein, 8 aa, Artificial Sequence
  Description of Artificial Sequence Synthetic peptide
GGTFSSYA

SEQ ID NO: 3 - protein, 17 aa, Artificial Sequence
  Description of Artificial Sequence Synthetic peptide
ISWVRQAPGQGLEWMGG

SEQ ID NO: 4 - protein, 8 aa, Artificial Sequence
  Description of Artificial Sequence Synthetic peptide
IIPIFGTA

SEQ ID NO: 5 - protein, 38 aa, Artificial Sequence
  Description of Artificial Sequence Synthetic polypeptide
NYAQKFQGRVTITADKSTSTAYMELSSLRSEDTAVYYC

SEQ ID NO: 6 - protein, 13 aa, Artificial Sequence
  Description of Artificial Sequence Synthetic peptide
ARGRQMFGAGIDF

SEQ ID NO: 7 - protein, 11 aa, Artificial Sequence
  Description of Artificial Sequence Synthetic peptide
WGPGTLVTVSS

SEQ ID NO: 8 - protein, 120 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGRQMFGAGIDFWGPGTLVTVSS

SEQ ID NO: 9 - protein, 25 aa, Artificial Sequence
  Description of Artificial Sequence Synthetic peptide
QVQLVQSGGEVKKPGASVKVSCKAS

SEQ ID NO: 10 - protein, 8 aa, Artificial Sequence
  Description of Artificial Sequence Synthetic peptide
GYTLSSHG

SEQ ID NO: 11 - protein, 25 aa, Artificial Sequence
  Description of Artificial Sequence Synthetic peptide
ITWVRQAPGQGLEWMGWGYTLSSHG

SEQ ID NO: 12 - protein, 8 aa, Artificial Sequence
  Description of Artificial Sequence Synthetic peptide
ISAHNGHA

SEQ ID NO: 13 - protein, 38 aa, Artificial Sequence
  Description of Artificial Sequence Synthetic polypeptide
SNAQKVEDRVTMTTDTSTNTAYMELRSLTADDTAVYYC

SEQ ID NO: 14 - protein, 13 aa, Artificial Sequence
  Description of Artificial Sequence Synthetic peptide
ARVHAALYYGMDV

SEQ ID NO: 15 - protein, 11 aa, Artificial Sequence
  Description of Artificial Sequence Synthetic peptide
WGQGTLVTVSS

SEQ ID NO: 16 - protein, 120 aa, Artificial Sequence
  Description of Artificial Sequence Synthetic polypeptide
QVQLVQSGGEVKKPGASVKVSCKASGYTLSSHGITWVRQAPGQGLEWMGWISAHNGHASNAQKVEDRVTMTTDTSTNTAYMELRSLTADDTAVYYCARVHAALYYGMDVWGQGTLVTVSS

SEQ ID NO: 17 - protein, 25 aa, Artificial Sequence
  Description of Artificial Sequence Synthetic peptide
NFMLTQPHSVSESPGKTVTISCTRS

SEQ ID NO: 18 - protein, 8 aa, Artificial Sequence
  Description of Artificial Sequence Synthetic peptide
SGSIDSNY

SEQ ID NO: 19 - protein, 17 aa, Artificial Sequence
  Description of Artificial Sequence Synthetic peptide
VQWYQQRPGSAPTTVIY

SEQ ID NO: 20 - protein, 3 aa, Artificial Sequence
  Description of Artificial Sequence Synthetic peptide
EDN

SEQ ID NO: 21 - protein, 38 aa, Artificial Sequence
  Description of Artificial Sequence Synthetic polypeptide
QRPSGVPDRFSGSIDSSSNSASLTISGLKTEDEADYYC

SEQ ID NO: 22 - protein, 11 aa, Artificial Sequence
  Description of Artificial Sequence Synthetic peptide
QSYDSNNRHVI

SEQ ID NO: 23 - protein, 10 aa, Artificial Sequence
  Description of Artificial Sequence Synthetic peptide
FGGGTKLTVL

SEQ ID NO: 24 - protein, 112 aa, Artificial Sequence
  Description of Artificial Sequence Synthetic polypeptide
NFMLTQPHSVSESPGKTVTISCTRSSGSIDSNYVQWYQQRPGSAPTTVIYEDNQRPSGVPDRFSGSIDSSSNSASLTISGLKTEDEADYYCQSYDSNNRHVIFGGGTKLTVL

SEQ ID NO: 25 - protein, 25 aa, Artificial Sequence
  Description of Artificial Sequence Synthetic peptide
SYELTQPPSVSLAPGQSARISCGGD

SEQ ID NO: 26 - protein, 6 aa, Artificial Sequence
  Description of Artificial Sequence Synthetic peptide
NIGSKG

SEQ ID NO: 27 - protein, 17 aa, Artificial Sequence
  Description of Artificial Sequence Synthetic peptide
VHWYQQKPGQAPVVVVY

SEQ ID NO: 28 - protein, 3 aa, Artificial Sequence
  Description of Artificial Sequence Synthetic peptide
DDR

SEQ ID NO: 29 - protein, 36 aa, Artificial Sequence
  Description of Artificial Sequence Synthetic polypeptide
DRPSGIPERFSGSNSGNTATLTISRVEAGDEADYYC

SEQ ID NO: 30 - protein, 11 aa, Artificial Sequence
  Description of Artificial Sequence Synthetic peptide
QVWDSGSDHWV

SEQ ID NO: 31 - protein, 108 aa, Artificial Sequence
  Description of Artificial Sequence Synthetic polypeptide
SYELTQPPSVSLAPGQSARISCGGDNIGSKGVHWYQQKPGQAPVVVVYDDRDRPSGIPERFSGSNSGNTATLTISRVEAGDEADYYCQVWDSGSDHWVFGGGTKLTVL

SEQ ID NO: 32 - protein, 25 aa, Artificial Sequence
  Description of Artificial Sequence Synthetic peptide
LPVLTQPPSVSAAPGQTARISCGGS

SEQ ID NO: 33 - protein, 6 aa, Artificial Sequence
  Description of Artificial Sequence Synthetic peptide
NIGDKG

SEQ ID NO: 34 - protein, 17 aa, Artificial Sequence
  Description of Artificial Sequence Synthetic peptide
VHWYQQKPGQAPVLVIY

SEQ ID NO: 35 - protein, 3 aa, Artificial Sequence
  Description of Artificial Sequence Synthetic peptide
DDS

SEQ ID NO: 36 - protein, 36 aa, Artificial Sequence
  Description of Artificial Sequence Synthetic polypeptide
SRRSGIPERFSGSHSGSAATLTISRVEAGDEADYYC

SEQ ID NO: 37 - protein, 11 aa, Artificial Sequence
  Description of Artificial Sequence Synthetic peptide
QVWDSSSDHWV

SEQ ID NO: 38 - protein, 108 aa, Artificial Sequence
  Description of Artificial Sequence Synthetic polypeptide
LPVLTQPPSVSAAPGQTARISCGGSNIGDKGVHWYQQKPGQAPVLVIYDDSDRPSGIPERFSGSNSGNTATLTISRVEAGDEADYYCQVWDSSSDHWVFGGGTKLTVL

SEQ ID NO: 39 - protein, 25 aa, Artificial Sequence
  Description of Artificial Sequence Synthetic peptide
SYELTQPPSVSVAPGQTARITCGGN

SEQ ID NO: 40 - protein, 6 aa, Artificial Sequence
  Description of Artificial Sequence Synthetic peptide
NIGNKG

SEQ ID NO: 41 - protein, 17 aa, Artificial Sequence
  Description of Artificial Sequence Synthetic peptide
VHWYQQKPGQAPVLVVY

SEQ ID NO: 42 - protein, 108 aa, Artificial Sequence
  Description of Artificial Sequence Synthetic polypeptide
SYELTQPPSVSVAPGQTARITCGGNNIGNKGVHWYQQKPGQAPVLVVYDDSDRPSGIPERFSGSNSGNTATLTISRVEAGDEADYYCQVWDSSSDHWVFGGGTKLTVL

SEQ ID NO: 43 - protein, 25 aa, Artificial Sequence
  Description of Artificial Sequence Synthetic peptide
LPVLTQPPSVSVALGQTARITCRGN

SEQ ID NO: 44 - protein, 6 aa, Artificial Sequence
  Description of Artificial Sequence Synthetic peptide
NIGGKG

SEQ ID NO: 45 - protein, 3 aa, Artificial Sequence
  Description of Artificial Sequence Synthetic peptide
DDY

SEQ ID NO: 46 - protein, 108 aa, Artificial Sequence
  Description of Artificial Sequence Synthetic polypeptide
LPVLTQPPSVSVALGQTARITCRGNNIGGKGVHWYQQKPGQAPVLVVYDDYSRRSGIPERFSGSHSGSAATLTISRVEAGDEADYYCQVWDSSSDHWVFGGGTKLTVL

SEQ ID NO: 47 - protein, 8 aa, Artificial Sequence
  Description of Artificial Sequence Synthetic peptide
  MOD_RES (2)..(2): Any amino acid
  MOD_RES (4)..(4): Any amino acid
  MOD_RES (7)..(8): Any amino acid
GXTXSSXX

SEQ ID NO: 48 - protein, 6 aa, Artificial Sequence
  Description of Artificial Sequence Synthetic peptide
  MOD_RES (4)..(4): Any amino acid
  MOD_RES (6)..(6): Any amino acid
NIGXKX

SEQ ID NO: 49 - protein, 3 aa, Artificial Sequence
  Description of Artificial Sequence Synthetic peptide
  MOD_RES (3)..(3): Any amino acid
DDX

SEQ ID NO: 50 - protein, 11 aa, Artificial Sequence
  Description of Artificial Sequence Synthetic peptide
  MOD_RES (6)..(6): Any amino acid
QVWDSXSDHWV

SEQ ID NO: 51 - protein, 8 aa, Artificial Sequence
  Description of Artificial Sequence Synthetic peptide
  MOD_RES (2)..(5): Any amino acid
  MOD_RES (7)..(7): Any amino acid
IXXXXGXA

SEQ ID NO: 52 - protein, 120 aa, Artificial Sequence
  Description of Artificial Sequence Synthetic polypeptide
EVQLVQSGGEVKKPGASVKVSCKASGYTLSSHGITWVRQAPGQGLEWMGWISAHNGHASNAQKVEDRVTMTTDTSTNTAYMELRSLTPDDTAVYYCARVHAALYYGMDVWGQGTLVTVSS

SEQ ID NO: 53 - protein, 108 aa, Artificial Sequence
  Description of Artificial Sequence Synthetic polypeptide
SYELTQPPSVSVAPRQTAKITCTRDNIESRSVNWYQQRAGQAPAVIVYDDTERPSGITVRYSGSNSGNTATLTISRVEAGDEADYYCQVWDSSGDLWVFGGGTKLTVL

SEQ ID NO: 54 - protein, 120 aa, Artificial Sequence
  Description of Artificial Sequence Synthetic polypeptide
QVQLVQSGGEVKKPGASVKVSCKASGYTLSSHGITWVRQAPGQGLEWMGWISAHNGHASNAQKVEDRVTMTTDTSTNTAYMELRSLTADDTAVYYCARVHAALYYGMDVWGQGTTVTVSS

SEQ ID NO: 55 - protein, 108 aa, Artificial Sequence
  Description of Artificial Sequence Synthetic polypeptide
QSVLTQPPSVSVAPGQTARITCGGNNIGSKGVHWYQQKPGQAPVLVVYDDSDRPSGIPERFSGSNSGNTATLTISRVEAGDEADYYCQVWDSSSDHWVFGGGTKLTVL

SEQ ID NO: 56 - protein, 120 aa, Artificial Sequence
  Description of Artificial Sequence Synthetic polypeptide
EVQLVQSGAEVKKPGASVKVSCKASGYTLSSHGITWVRQAPGQGLEWMGWISAHNGHASNAQKVEDRVTMTTDTSTNTAYMELRSLTPDDTAVYYCARVHAALYYGMDVWGQGTLVTVSS

SEQ ID NO: 57 - protein, 108 aa, Artificial Sequence
  Description of Artificial Sequence Synthetic polypeptide
LPVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVL

SEQ ID NO: 58 - protein, 17 aa, Artificial Sequence
  Description of Artificial Sequence Synthetic peptide
ITWVRQAPGQGLEWMGW

SEQ ID NO: 59 - protein, 108 aa, Artificial Sequence; identical to SEQ ID NO: 55
  Description of Artificial Sequence Synthetic polypeptide

SEQ ID NO: 60 - protein, 126 aa, Artificial Sequence
  Description of Artificial Sequence Synthetic polypeptide
QVQLVQSGGGLVKPGGSLRLSCVGSDFAFSSAWMNWVRQAPGKGLEWVGRIKSKTDGETTDYAAPVKDRFIISRDDSKNTLYLEMNSLKTEDTGVYYCTTGGLGLVYPYYNYIDVWGEGTTVTVSS

SEQ ID NO: 61 - protein, 110 aa, Artificial Sequence
  Description of Artificial Sequence Synthetic polypeptide
LPVLTQPPSASGTPGQRVTISCSGSSSNIGSNYVYWYQQLPGTAPKLLIYRNNQRPSGVPDRFSGSKSGTSASLAISGLRSEDEADYYCAAWDDSLNGLVFGTGTRVTVL

SEQ ID NO: 62 - protein, 120 aa, Artificial Sequence
  Description of Artificial Sequence Synthetic polypeptide
QVQLVQSGAEVKKPGASVKVSCKASGYTFTSYGISWVRQAPGQGLEWMGWTSPHNGLTAFAQILEGRVTMTTDTSTNTAYMELRNLTFDDTAVYFCAKVHPVFSYALDVWGQGTLVTVSS

SEQ ID NO: 63 - protein, 110 aa, Artificial Sequence
  Description of Artificial Sequence Synthetic polypeptide
NFMLTQPHSVSESPGKTVTISCTRSSGSIASNYVQWYQQRPGSSPTTVIYEDNQRPSGVPDRFSGSIDTSSNSASLTISGLKTKDEADYYCQSYDGITVIFGGGTKLTVL

SEQ ID NO: 64 - protein, 118 aa, Artificial Sequence
  Description of Artificial Sequence Synthetic polypeptide
QVQLVQSGAEVKKPGSSVKVSCKASGGTFSRYALTWVRQAPGQGLEWMGGIIPIFGRANYAQKFQGRVTITADKSTSTAYMELGSLTSDDTAVYYCAEEGAFNSLAIWGQGTMVTVSS

SEQ ID NO: 65 - protein, 111 aa, Artificial Sequence
  Description of Artificial Sequence Synthetic polypeptide
NFMLTQPHSVSESPGKTVTISCTRSSGSIASNYVQWYQQRPGSAPTTVIFADNQRPSGVPARFSGSIDSSSNSASLTISGLKTEDEADYYCQSYDSSNHWVFGGGTQLTVL

SEQ ID NO: 66 - protein, 120 aa, Artificial Sequence
  Description of Artificial Sequence Synthetic polypeptide
QVQLVQSGGEVKKPGASVKVSCKASGYTLSSHGITWVRQAPGQGLEWMGWISAHNGHASNAQKVEDRVTMTTDTSTNTAYMELRSLTPDDTAVYYCARVHAALYYGMDVWGQGTLVTVSS

SEQ ID NO: 67 - protein, 108 aa, Artificial Sequence
  Description of Artificial Sequence Synthetic polypeptide
LPVLTQPPSVSVAPGQTARITCGGNNIGSKSVHWYQQKPGQAPVLVVYDDSDRPSGIPERFSGSNSGNTATLTINRVEAGDEADYYCQVWDSSSDHWVFGGGTKLTVL

SEQ ID NO: 68 - protein, 120 aa, Artificial Sequence
  Description of Artificial Sequence Synthetic polypeptide
QVQLVQSGAEVKKPGASVKVSCKASGYTFTSYGISWVRQAPGQGLEWMGWISAYNGHASNAQKVEDRVTMTTDTSTNTAYMELRNLTTDDTAVYYCARVHAALYYGMDVWGQGTTVTVSS

SEQ ID NO: 69 - protein, 108 aa, Artificial Sequence
  Description of Artificial Sequence Synthetic polypeptide
SYELTQPPSVSVAPGQTARITCGGDNIGSKGVHWYQQTPGQAPVLVVYDDSDRPSGIPERFSGSKSGNTATLTISRVEAGDEADYYCQVWDSRSDHWVFGGGTRVTVL

SEQ ID NO: 70 - protein, 121 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGSGYDSAGMDDWGQGTLVTVSS

SEQ ID NO: 71 - protein, 110 aa, Artificial Sequence
  Description of Artificial Sequence Synthetic polypeptide
QPGLTQPPSASGTPGQTVTISCSGSRSNIGSNYVYWYQQFPGTAPKLLIFSNNQRPSGVPDRFSGSKSGTSASLAISGLRSEDEADYYCAVWDDSLSGVVFGGGTKLTVL

SEQ ID NO: 72 - protein, 116 aa, Artificial Sequence
  Description of Artificial Sequence Synthetic polypeptide
QVQLVQSGGGVVQPGRSLRLSCAASGFTFSSYAIHWVRQAPGKGLEWITTISYDGSNKYYADSVKGRFTISRDNPKNTLYLQMNSLRAEDTAVYYCARGFGGPDYWGQGTLVTVSS

SEQ ID NO: 73 - protein, 115 aa, Artificial Sequence
  Description of Artificial Sequence Synthetic polypeptide
LPVLTQPSSLSASPGASASLTCTLRSGINVGTYRIYWYQQKPGSPPQYLLRYKSDSDKQQGSGVPSRFSGSKDASANAGILLISGLQSEDEADYYCMIWHSSAYVFGTGTKVTVL

SEQ ID NO: 74 - protein, 120 aa, Artificial Sequence
  Description of Artificial Sequence Synthetic polypeptide
QVQLVQSGVEMKKPGASVRVSCKGSGYTFSSYGISWVRQAPGQGLEWMGWISAHNGHASNAQKLEDRVTMTTDTSTNTAYMELRSLTSDDTAVYYCARVHGALYYGMDVWGQGTTVTVSS

SEQ ID NO: 75 - protein, 108 aa, Artificial Sequence
  Description of Artificial Sequence Synthetic polypeptide
SYELTQPPSVSVAPGQTARIPCGANNIGGKSVHWYQQKPGQAPVLVVYDDRDRPSGIPERFSGSNSGNTATLTISRVEAGDEADYYCQVWDSSSDHWVFGGGTKLTVL

SEQ ID NO: 76 - protein, 120 aa, Artificial Sequence; identical to SEQ ID NO: 66
  Description of Artificial Sequence Synthetic polypeptide

SEQ ID NO: 77 - protein, 108 aa, Artificial Sequence
  Description of Artificial Sequence Synthetic polypeptide
QPVLTQPPSVSVAPGQTARITCGGDNIGSKGVHWLQQKPGQAPVLVVYDDRDRPSGIPERFSGSNSGSTATLTISRVEAGDEADYYCQVWDSSSDHWVFGGGTKLTVL

SEQ ID NO: 78 - protein, 121 aa, Artificial Sequence
  Description of Artificial Sequence Synthetic polypeptide
EVQLVQSGAEVKKPGSSVRVSCKASGGTFSSYAISWVRQAPGQGLEWMGRIIPILGIANYAQKFQGRVTITADKSTSTAYMELSSLRSEDTAVYYCASGSIVGAAYAFDIWGQGTTVTVSS

SEQ ID NO: 79 - protein, 108 aa, Artificial Sequence
  Description of Artificial Sequence Synthetic polypeptide
QSVLTQPPSVSVAPGKAANLNCGGKNIGGRVVHWYQQRPGQAPVLVIYDDTDRPSGIPERFSGSNSGNTATLTITDVEVGDEADYYCQVWDSRSDHPVFGGGTTLTVL

SEQ ID NO: 80 - protein, 118 aa, Artificial Sequence
  Description of Artificial Sequence Synthetic polypeptide
QVQLVQSGGGLVKPGGSLRLSCAASGFTFSSYSMNWVRQAPGKGLVWISRIISDGSATTYADSVKGRFTISRDNAKNTLYLQMNSLRAEDTGVYYCARDRSEGGFDPWGQGTLVTVSS

SEQ ID NO: 81 - protein, 110 aa, Artificial Sequence
  Description of Artificial Sequence Synthetic polypeptide
SSELTQDPAVSVALGQTVRITCQGDSLRSYYASWYQQKPGQAPVLVIYGKNNRPSGIPDRFSGSTSGNTASLTITGAQAEDEADYYCNSRDISDNQWQWIFGGGTKLAVL

SEQ ID NO: 82 - protein, 118 aa, Artificial Sequence; identical to SEQ ID NO: 64
  Description of Artificial Sequence Synthetic polypeptide

SEQ ID NO: 83 - protein, 111 aa, Artificial Sequence
  Description of Artificial Sequence Synthetic polypeptide
NFMLTQPHSVSGSPGETVTISCTRSSGSIASHFVQWYQQRPGSSPTTVIFGDDQRPSGVPDRISGSIDTSSNSASLSISGLKTEDEADYYCQSYDSSNHVVFGGGTKLTVL

SEQ ID NO: 84 - protein, 8 aa, Artificial Sequence
  Description of Artificial Sequence Synthetic peptide
DFAFSSAW

SEQ ID NO: 85 - protein, 8 aa, Artificial Sequence
  Description of Artificial Sequence Synthetic peptide
GYTFTSYG

SEQ ID NO: 86 - protein, 8 aa, Artificial Sequence
  Description of Artificial Sequence Synthetic peptide
GGTFSRYA

SEQ ID NO: 87 - protein, 8 aa, Artificial Sequence
  Description of Artificial Sequence Synthetic peptide
GGTFSSYA

SEQ ID NO: 88 - protein, 8 aa, Artificial Sequence
  Description of Artificial Sequence Synthetic peptide
GFTFSSYA

SEQ ID NO: 89 - protein, 8 aa, Artificial Sequence
  Description of Artificial Sequence Synthetic peptide
GYTFSSYG

SEQ ID NO: 90 - protein, 8 aa, Artificial Sequence
  Description of Artificial Sequence Synthetic peptide
GFTFSSYS

SEQ ID NO: 91 - protein, 10 aa, Artificial Sequence
  Description of Artificial Sequence Synthetic peptide
IKSKTDGETT

SEQ ID NO: 92 - protein, 8 aa, Artificial Sequence
  Description of Artificial Sequence Synthetic peptide
TSPHNGLT

SEQ ID NO: 93 - protein, 8 aa, Artificial Sequence
  Description of Artificial Sequence Synthetic peptide
IIPIFGRA

SEQ ID NO: 94 - protein, 8 aa, Artificial Sequence
  Description of Artificial Sequence Synthetic peptide
ISAYNGHA

SEQ ID NO: 95 - protein, 8 aa, Artificial Sequence
  Description of Artificial Sequence Synthetic peptide
IIPIFGTA

SEQ ID NO: 96 - protein, 8 aa, Artificial Sequence
  Description of Artificial Sequence Synthetic peptide
ISYDGSNK

SEQ ID NO: 97 - protein, 8 aa, Artificial Sequence
  Description of Artificial Sequence Synthetic peptide
IIPILGIA

SEQ ID NO: 98 - protein, 8 aa, Artificial Sequence
  Description of Artificial Sequence Synthetic peptide
IISDGSAT

SEQ ID NO: 99 - protein, 17 aa, Artificial Sequence
  Description of Artificial Sequence Synthetic peptide
TTGGLGLVYPYYNYIDV

SEQ ID NO: 100 - protein, 13 aa, Artificial Sequence
  Description of Artificial Sequence Synthetic peptide
AKVHPVFSYALDV

SEQ ID NO: 101 - protein, 11 aa, Artificial Sequence
  Description of Artificial Sequence Synthetic peptide
AEEGAFNSLAI

SEQ ID NO: 102 - protein, 14 aa, Artificial Sequence
  Description of Artificial Sequence Synthetic peptide
ARDGSGYDSAGMDD

SEQ ID NO: 103 - protein, 9 aa, Artificial Sequence
  Description of Artificial Sequence Synthetic peptide
ARGFGGPDY

SEQ ID NO: 104 - protein, 13 aa, Artificial Sequence
  Description of Artificial Sequence Synthetic peptide
ARVHGALYYGMDV

SEQ ID NO: 105 - protein, 14 aa, Artificial Sequence
  Description of Artificial Sequence Synthetic peptide
ASGSIVGAAYAFDI

SEQ ID NO: 106 - protein, 11 aa, Artificial Sequence
  Description of Artificial Sequence Synthetic peptide
ARDRSEGGFDP

SEQ ID NO: 107 - protein, 11 aa, Artificial Sequence
  Description of Artificial Sequence Synthetic peptide
AEEGAFNSLAI

SEQ ID NO: 108 - protein, 6 aa, Artificial Sequence
  Description of Artificial Sequence Synthetic peptide
NIESRS

SEQ ID NO: 109 - protein, 6 aa, Artificial Sequence
  Description of Artificial Sequence Synthetic peptide
NIGSKS

SEQ ID NO: 110 - protein, 8 aa, Artificial Sequence
  Description of Artificial Sequence Synthetic peptide
SSNIGSNY

SEQ ID NO: 111 - protein, 8 aa, Artificial Sequence
  Description of Artificial Sequence Synthetic peptide
SGSIASNY

SEQ ID NO: 112 - protein, 8 aa, Artificial Sequence
  Description of Artificial Sequence Synthetic peptide
RSNIGSNY

SEQ ID NO: 113 - protein, 9 aa, Artificial Sequence
  Description of Artificial Sequence Synthetic peptide
SGINVGTYR

SEQ ID NO: 114 - protein, 6 aa, Artificial Sequence
  Description of Artificial Sequence Synthetic peptide
NIGGKS

SEQ ID NO: 115 - protein, 6 aa, Artificial Sequence
  Description of Artificial Sequence Synthetic peptide
NIGGRV

SEQ ID NO: 116 - protein, 6 aa, Artificial Sequence
  Description of Artificial Sequence Synthetic peptide
SLRSYY

SEQ ID NO: 117 - protein, 8 aa, Artificial Sequence
  Description of Artificial Sequence Synthetic peptide
SGSIASHF

SEQ ID NO: 118 - protein, 3 aa, Artificial Sequence
  Description of Artificial Sequence Synthetic peptide
DDT

SEQ ID NO: 119 - protein, 3 aa, Artificial Sequence
  Description of Artificial Sequence Synthetic peptide
RNN

SEQ ID NO: 120 - protein, 3 aa, Artificial Sequence
  Description of Artificial Sequence Synthetic peptide
ADN

SEQ ID NO: 121 - protein, 3 aa, Artificial Sequence
  Description of Artificial Sequence Synthetic peptide
SNN

SEQ ID NO: 122 - protein, 7 aa, Artificial Sequence
  Description of Artificial Sequence Synthetic peptide
YKSDSDK

SEQ ID NO: 123 - protein, 3 aa, Artificial Sequence
  Description of Artificial Sequence Synthetic peptide
DDT

SEQ ID NO: 124 - protein, 3 aa, Artificial Sequence
  Description of Artificial Sequence Synthetic peptide
GKN

SEQ ID NO: 125 - protein, 3 aa, Artificial Sequence
  Description of Artificial Sequence Synthetic peptide
GDD

SEQ ID NO: 126 - protein, 11 aa, Artificial Sequence
  Description of Artificial Sequence Synthetic peptide
QVWDSSGDLWV

SEQ ID NO: 127 - protein, 11 aa, Artificial Sequence
  Description of Artificial Sequence Synthetic peptide
AAWDDSLNGLV

SEQ ID NO: 128 - protein, 9 aa, Artificial Sequence
  Description of Artificial Sequence Synthetic peptide
QSYDGITVI

SEQ ID NO: 129 - protein, 10 aa, Artificial Sequence
  Description of Artificial Sequence Synthetic peptide
QSYDSSNHWV

SEQ ID NO: 130 - protein, 11 aa, Artificial Sequence
  Description of Artificial Sequence Synthetic peptide
QVWDSRSDHWV

SEQ ID NO: 131 - protein, 11 aa, Artificial Sequence
  Description of Artificial Sequence Synthetic peptide
AVWDDSLSGVV

SEQ ID NO: 132 - protein, 9 aa, Artificial Sequence
  Description of Artificial Sequence Synthetic peptide
MIWHSSAYV

SEQ ID NO: 133 - protein, 11 aa, Artificial Sequence
  Description of Artificial Sequence Synthetic peptide
QVWDSRSDHPV

SEQ ID NO: 134 - protein, 13 aa, Artificial Sequence
  Description of Artificial Sequence Synthetic peptide
NSRDISDNQWQWI

SEQ ID NO: 135 - protein, 10 aa, Artificial Sequence
  Description of Artificial Sequence Synthetic peptide
QSYDSSNHVV

SEQ ID NO: 136 - protein, 25 aa, Artificial Sequence
  Description of Artificial Sequence Synthetic peptide
EVQLVQSGGEVKKPGASVKVSCKAS

SEQ ID NO: 137 - protein, 17 aa, Artificial Sequence
  Description of Artificial Sequence Synthetic peptide
IHWVRQAPGKGLEWITT

SEQ ID NO: 138 - protein, 25 aa, Artificial Sequence
  Description of Artificial Sequence Synthetic peptide
EVQLVQSGAEVKKPGASVKVSCKAS

SEQ ID NO: 139 - protein, 25 aa, Artificial Sequence
  Description of Artificial Sequence Synthetic peptide
QVQLVQSGGGLVKPGGSLRLSCVGS

SEQ ID NO: 140 - protein, 25 aa, Artificial Sequence [sequence omitted]

SEQ ID NO: 141 - protein, 25 aa, Artificial Sequence [sequence omitted]

SEQ ID NO: 142 - protein, 25 aa, Artificial Sequence
  Description of Artificial Sequence Synthetic peptide
QVQLVQSGGGVVQPGRSLRLSCAAS

SEQ ID NO: 143 - protein, 25 aa, Artificial Sequence
  Description of Artificial Sequence Synthetic peptide
QVQLVQSGVEMKKPGASVRVSCKGS

SEQ ID NO: 144 - protein, 25 aa, Artificial Sequence
  Description of Artificial Sequence Synthetic peptide
EVQLVQSGAEVKKPGSSVRVSCKAS

SEQ ID NO: 145 - protein, 25 aa, Artificial Sequence
  Description of Artificial Sequence Synthetic peptide
QVQLVQSGGGLVKPGGSLRLSCAAS

SEQ ID NO: 146 - protein, 17 aa, Artificial Sequence
  Description of Artificial Sequence Synthetic peptide
MNWVRQAPGKGLEWVGR

SEQ ID NO: 147 - protein, 17 aa, Artificial Sequence
  Description of Artificial Sequence Synthetic peptide
ISWVRQAPGQGLEWMGW

SEQ ID NO: 148 - protein, 17 aa, Artificial Sequence
  Description of Artificial Sequence Synthetic peptide
LTWVRQAPGQGLEWMGG

SEQ ID NO: 149 - protein, 17 aa, Artificial Sequence
  Description of Artificial Sequence Synthetic peptide
ISWVRQAPGQGLEWMGR

SEQ ID NO: 150 - protein, 17 aa, Artificial Sequence
  Description of Artificial Sequence Synthetic peptide
MNWVRQAPGKGLVWISR

SEQ ID NO: 151 - protein, 38 aa, Artificial Sequence
  Description of Artificial Sequence Synthetic polypeptide
SNAQKVEDRVTMTTDTSTNTAYMELRSLTPDDTAVYYC

SEQ ID NO: 152 - protein, 38 aa, Artificial Sequence
  Description of Artificial Sequence Synthetic polypeptide
DYAAPVKDRFIISRDDSKNTLYLEMNSLKTEDTGVYYC

SEQ ID NO: 153 - protein, 38 aa, Artificial Sequence
  Description of Artificial Sequence Synthetic polypeptide
AFAQILEGRVTMTTDTSTNTAYMELRNLTFDDTAVYFC

SEQ ID NO: 154 - protein, 38 aa, Artificial Sequence
  Description of Artificial Sequence Synthetic polypeptide
NYAQKFQGRVTITADKSTSTAYMELGSLTSDDTAVYYC

SEQ ID NO: 155 - protein, 38 aa, Artificial Sequence
  Description of Artificial Sequence Synthetic polypeptide
SNAQKVEDRVTMTTDTSTNTAYMELRNLTTDDTAVYYC

SEQ ID NO: 156 - protein, 38 aa, Artificial Sequence
  Description of Artificial Sequence Synthetic polypeptide
NYAQKFQGRVTITADESTSTAYMELSSLRSEDTAVYYC

SEQ ID NO: 157 - protein, 38 aa, Artificial Sequence
  Description of Artificial Sequence Synthetic polypeptide
YYADSVKGRFTISRDNPKNTLYLQMNSLRAEDTAVYYC

SEQ ID NO: 158 - protein, 38 aa, Artificial Sequence
  Description of Artificial Sequence Synthetic polypeptide
SNAQKLEDRVTMTTDTSTNTAYMELRSLTSDDTAVYYC

SEQ ID NO: 159 - protein, 38 aa, Artificial Sequence
  Description of Artificial Sequence Synthetic polypeptide
TYADSVKGRFTISRDNAKNTLYLQMNSLRAEDTGVYYC

SEQ ID NO: 160 - protein, 11 aa, Artificial Sequence
  Description of Artificial Sequence Synthetic peptide
WGQGTTVTVSS

SEQ ID NO: 161 - protein, 11 aa, Artificial Sequence
  Description of Artificial Sequence Synthetic peptide
WGEGTTVTVSS

SEQ ID NO: 162 - protein, 11 aa, Artificial Sequence
  Description of Artificial Sequence Synthetic peptide
WGQGTMVTVSS

SEQ ID NO: 163 - protein, 25 aa, Artificial Sequence
  Description of Artificial Sequence Synthetic peptide
SYELTQPPSVSVAPRQTAKITCTRD

SEQ ID NO: 164 - protein, 25 aa, Artificial Sequence
  Description of Artificial Sequence Synthetic peptide
QSVLTQPPSVSVAPGQTARITCGGN

SEQ ID NO: 165 - protein, 25 aa, Artificial Sequence
  Description of Artificial Sequence Synthetic peptide
LPVLTQPPSVSVAPGQTARITCGGN

SEQ ID NO: 166 - protein, 25 aa, Artificial Sequence
  Description of Artificial Sequence Synthetic peptide
LPVLTQPPSASGTPGQRVTISCSGS

SEQ ID NO: 167 - protein, 25 aa, Artificial Sequence
  Description of Artificial Sequence Synthetic peptide
NFMLTQPHSVSESPGKTVTISCTRS

SEQ ID NO: 168 - protein, 25 aa, Artificial Sequence
  Description of Artificial Sequence Synthetic peptide
SYELTQPPSVSVAPGQTARITCGGD

SEQ ID NO: 169 - protein, 25 aa, Artificial Sequence
  Description of Artificial Sequence Synthetic peptide
QPGLTQPPSASGTPGQTVTISCSGS

SEQ ID NO: 170 - protein, 25 aa, Artificial Sequence
  Description of Artificial Sequence Synthetic peptide
LPVLTQPSSLSASPGASASLTCTLR

SEQ ID NO: 171 - protein, 25 aa, Artificial Sequence
  Description of Artificial Sequence Synthetic peptide
SYELTQPPSVSVAPGQTARIPCGAN

SEQ ID NO: 172 - protein, 25 aa, Artificial Sequence
  Description of Artificial Sequence Synthetic peptide
QPVLTQPPSVSVAPGQTARITCGGD

SEQ ID NO: 173 - protein, 25 aa, Artificial Sequence
  Description of Artificial Sequence Synthetic peptide
QSVLTQPPSVSVAPGKAANLNCGGK

SEQ ID NO: 174 - protein, 25 aa, Artificial Sequence
  Description of Artificial Sequence Synthetic peptide
SSELTQDPAVSVALGQTVRITCQGD

SEQ ID NO: 175 - protein, 25 aa, Artificial Sequence
  Description of Artificial Sequence Synthetic peptide
NFMLTQPHSVSGSPGETVTISCTRS

SEQ ID NO: 176 - protein, 17 aa, Artificial Sequence
  Description of Artificial Sequence Synthetic peptide
VNWYQQRAGQAPAVIVY

SEQ ID NO: 177 - protein, 17 aa, Artificial Sequence
  Description of Artificial Sequence Synthetic peptide
VYWYQQLPGTAPKLLIY

SEQ ID NO: 178 - protein, 17 aa, Artificial Sequence
  Description of Artificial Sequence Synthetic peptide
VQWYQQRPGSSPTTVIY

SEQ ID NO: 179 - protein, 17 aa, Artificial Sequence
  Description of Artificial Sequence Synthetic peptide
VQWYQQRPGSAPTTVIF

SEQ ID NO: 180 - protein, 17 aa, Artificial Sequence
  Description of Artificial Sequence Synthetic peptide
VHWYQQTPGQAPVLVVY

SEQ ID NO: 181 - protein, 17 aa, Artificial Sequence
  Description of Artificial Sequence Synthetic peptide
VYWYQQFPGTAPKLLIF

SEQ ID NO: 182 - protein, 17 aa, Artificial Sequence
  Description of Artificial Sequence Synthetic peptide
IYWYQQKPGSPPQYLLR

SEQ ID NO: 183 - protein, 17 aa, Artificial Sequence
  Description of Artificial Sequence Synthetic peptide
VHWLQQKPGQAPVLVVY

SEQ ID NO: 184 - protein, 17 aa, Artificial Sequence
  Description of Artificial Sequence Synthetic peptide
VHWYQQRPGQAPVLVIY

SEQ ID NO: 185 - protein, 17 aa, Artificial Sequence
  Description of Artificial Sequence Synthetic peptide
ASWYQQKPGQAPVLVIY

SEQ ID NO: 186 - protein, 17 aa, Artificial Sequence
  Description of Artificial Sequence Synthetic peptide
VQWYQQRPGSSPTTVIF

SEQ ID NO: 187 - protein, 36 aa, Artificial Sequence
  Description of Artificial Sequence Synthetic polypeptide
ERPSGITVRYSGSNSGNTATLTISRVEAGDEADYYC

SEQ ID NO: 188 - protein, 36 aa, Artificial Sequence
  Description of Artificial Sequence Synthetic polypeptide
QRPSGVPDRFSGSKSGTSASLAISGLRSEDEADYYC

SEQ ID NO: 189 - protein, 38 aa, Artificial Sequence
  Description of Artificial Sequence Synthetic polypeptide
QRPSGVPDRFSGSIDTSSNSASLTISGLKTKDEADYYC

SEQ ID NO: 190 - protein, 38 aa, Artificial Sequence
  Description of Artificial Sequence Synthetic polypeptide
QRPSGVPARFSGSIDSSSNSASLTISGLKTEDEADYYC

SEQ ID NO: 191 - protein, 36 aa, Artificial Sequence
  Description of Artificial Sequence Synthetic polypeptide
DRPSGIPERFSGSNSGNTATLTINRVEAGDEADYYC

SEQ ID NO: 192 - protein, 36 aa, Artificial Sequence
  Description of Artificial Sequence Synthetic polypeptide
DRPSGIPERFSGSKSGNTATLTISRVEAGDEADYYC

SEQ ID NO: 193 - protein, 38 aa, Artificial Sequence
  Description of Artificial Sequence Synthetic polypeptide
QQGSGVPSRFSGSKDASANAGILLISGLQSEDEADYYC

SEQ ID NO: 194 - protein, 36 aa, Artificial Sequence
  Description of Artificial Sequence Synthetic polypeptide
DRPSGIPERFSGSNSGSTATLTISRVEAGDEADYYC

SEQ ID NO: 195 - protein, 36 aa, Artificial Sequence
  Description of Artificial Sequence Synthetic polypeptide
DRPSGIPERFSGSNSGNTATLTITDVEVGDEADYYC

SEQ ID NO: 196 - protein, 36 aa, Artificial Sequence
  Description of Artificial Sequence Synthetic polypeptide
NRPSGIPDRFSGSTSGNTASLTITGAQAEDEADYYC

SEQ ID NO: 197 - protein, 38 aa, Artificial Sequence
  Description of Artificial Sequence Synthetic polypeptide
QRPSGVPDRISGSIDTSSNSASLSISGLKTEDEADYYC

SEQ ID NO: 198 - protein, 10 aa, Artificial Sequence
  Description of Artificial Sequence Synthetic peptide
FGTGTRVTVL

SEQ ID NO: 199 - protein, 10 aa, Artificial Sequence
  Description of Artificial Sequence Synthetic peptide
FGGGTQLTVL

SEQ ID NO: 200 - protein, 10 aa, Artificial Sequence
  Description of Artificial Sequence Synthetic peptide
FGGGTRVTVL

SEQ ID NO: 201 - protein, 10 aa, Artificial Sequence
  Description of Artificial Sequence Synthetic peptide
FGTGTKVTVL

SEQ ID NO: 202 - protein, 10 aa, Artificial Sequence
  Description of Artificial Sequence Synthetic peptide
FGGGTTLTVL

SEQ ID NO: 203 - protein, 10 aa, Artificial Sequence
  Description of Artificial Sequence Synthetic peptide
FGGGTKLAVL

SEQ ID NO: 204 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 205 - protein, 8 aa, Artificial Sequence
  Description of Artificial Sequence Synthetic peptide
  MOD_RES (2)..(2): Any amino acid
  MOD_RES (4)..(8): Any amino acid
GXTXXXXX

SEQ ID NO: 206 - protein, 8 aa, Artificial Sequence
  Description of Artificial Sequence Synthetic peptide
  MOD_RES (2)..(2): Any amino acid
  MOD_RES (5)..(6): Any amino acid
  MOD_RES (8)..(8): Any amino acid
GXTFXXYX

SEQ ID NO: 207 - protein, 8 aa, Artificial Sequence
  Description of Artificial Sequence Synthetic peptide
  MOD_RES (3)..(3): Any amino acid
  MOD_RES (7)..(7): Any amino acid
IIXIFGXA

SEQ ID NO: 208 - protein, 8 aa, Artificial Sequence
  Description of Artificial Sequence Synthetic peptide
  MOD_RES (2)..(3): Any amino acid
  MOD_RES (5)..(5): Any amino acid
SXXIXSNY

SEQ ID NO: 209 - protein, 3 aa, Artificial Sequence
  Description of Artificial Sequence Synthetic peptide
  MOD_RES (1)..(1): Any amino acid
XDN

SEQ ID NO: 210 - protein, 3 aa, Artificial Sequence
  Description of Artificial Sequence Synthetic peptide
  MOD_RES (1)..(1): Any amino acid
XNN

SEQ ID NO: 211 - DNA, 355 bp, Artificial Sequence
  Description of Artificial Sequence Synthetic polynucleotide
caggtgcagctggtgcagtctgggggaggcttggtacagcctggggggtccctgagactctcctgtgcagcctctggattcacctttagcagccatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaatcggtacggcggatgcttttgatatctggggccaagggaccacggtcaccgtctcctcag

SEQ ID NO: 212 - DNA, 331 bp, Artificial Sequence
  Description of Artificial Sequence Synthetic polynucleotide
cagtctgccctgactcagccaccctcagtgtctgggacccccggacagagggtcaccatctcttgttctggaggcgtccccaacatcggaagtaatcctgtaaactggtacctccaccgcccaggaacggcccccaaactcctcatctataatagcaatcagtggccctcaggggtccctgaccgattttctggctccaggtctggcacctcagcctccctggccatcagtgggctccagtctgaggatgaggctgattattactgtgcagcatgggatgacagcctggatggtctggttttcggcggagggaccaagttgaccgtcctag

SEQ ID NO: 213 - protein, 118 aa, Artificial Sequence
  Description of Artificial Sequence Synthetic polypeptide
QVQLVQSGGGLVQPGGSLRLSCAASGFTFSSHAMSWVRQAPGKGLEWVSAISGSGGSTYYADSVKGRFTISRDNSKNTLYLQMNSLRAEDTAVYYCAKIGTADAFDIWGQGTTVTVSS

SEQ ID NO: 214 - protein, 110 aa, Artificial Sequence
  Description of Artificial Sequence Synthetic polypeptide
QSALTQPPSVSGTPGQRVTISCSGGVPNIGSNPVNWYLHRPGTAPKLLIYNSNQWPSGVPDRFSGSRSGTSASLAISGLQSEDEADYYCAAWDDSLDGLVFGGGTKLTVL

SEQ ID NO: 215 - DNA, 285 bp, Homo sapiens
accaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaa

SEQ ID NO: 216 - DNA, 48 bp, Homo sapiens
gcagagcccaaatcttgtgacaaaactcacacatgcccaccgtgccca

SEQ ID NO: 217 - DNA, 330 bp, Homo sapiens
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 218 - DNA, 324 bp, Homo sapiens
gggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 219 - DNA, 321 bp, Homo sapiens
ggtcagcccaaggctgccccctcggtcactctgttcccgccctcctctgaggagcttcaagccaacaaggccacactggtgtgtctcataagtgacttctacccgggagccgtgacagtggcctggaaggcagatggcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 220 - protein, 97 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKK

SEQ ID NO: 221 - protein, 16 aa, Homo sapiens
AEPKSCDKTHTCPPCP

SEQ ID NO: 222 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 223 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 106 aa, Homo sapiens
GQPKAAPSVTLFPPSSEELQANKATLVCLISDFYPGAVTVAWKADGSPVKAGVETTTPSKQSNNKYAASSYLSLTPEQWKSHRSYSCQVTHEGSTVEKTVAPTECS

SEQ ID NO: 225 - DNA, 330 bp, Artificial Sequence
  Description of Artificial Sequence Synthetic polynucleotide
gcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 226 - DNA, 324 bp, Artificial Sequence
  Description of Artificial Sequence Synthetic polynucleotide
gggcagccccgaaagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatggagctctgcacaaccactacacgcagaagagcctctccctgtctccgggtaaatga

SEQ ID NO: 227 - DNA, 1095 bp, Artificial Sequence
  Description of Artificial Sequence Synthetic polynucleotide
ggtcagcccaaggctgccccctcggtcactctgttcccgccctcctctgaggagcttcaagccaacaaggccacactggtgtgtctcataagtgacttctacccgggagccgtgacagtggcctggaaggcagatggcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcaggtggcggcggttccggaggtggtggttcacaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaggacacggccgtctattactgtgcgagagggcgtcaaatgttcggtgcgggaattgatttctggggcccgggcaccctggtcaccgtctcctcaggtggcggcggttccggaggtggtggttctggcggtggtggcagcatcaattttatgctgactcagccccactctgtgtcggagtctccggggaagacggtaaccatctcctgcacccgcagcagtggcagcattgacagcaactatgtgcagtggtaccagcagcgcccgggcagcgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcaacaatcgtcatgtgatattcggcggagggaccaagctgaccgtcctaggt

SEQ ID NO: 228 - protein, 110 aa, Artificial Sequence
  Description of Artificial Sequence Synthetic polypeptide
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 229 - protein, 107 aa, Artificial Sequence
  Description of Artificial Sequence Synthetic polypeptide
GQPRKPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 230 - protein, 365 aa, Artificial Sequence
  Description of Artificial Sequence Synthetic polypeptide
GQPKAAPSVTLFPPSSEELQANKATLVCLISDFYPGAVTVAWKADGSPVKAGVETTTPSKQSNNKYAASSYLSLTPEQWKSHRSYSCQVTHEGSTVEKTVAPTECSGGGGSGGGGSQVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGRQMFGAGIDFWGPGTLVTVSSGGGGSGGGGSGGGGSINFMLTQPHSVSESPGKTVTISCTRSSGSIDSNYVQWYQQRPGSAPTTVIYEDNQRPSGVPDRFSGSIDSSSNSASLTISGLKTEDEADYYCQSYDSNNRHVIFGGGTKLTVLG

SEQ ID NO: 231 - DNA, 1125 bp, Artificial Sequence
  Description of Artificial Sequence Synthetic polynucleotide
ggtcagcccaaggctgccccctcggtcactctgttcccgccctcctctgaggagcttcaagccaacaaggccacactggtgtgtctcataagtgacttctacccgggagccgtgacagtggcctggaaggcagatggcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcaggtggcggcggttccggaggtggtggttcatcgatggcccaggtgcagctggtgcagtctggaggtgaggtgaagaagccgggggcctcagtgaaggtctcctgcaaggcttctggttacaccttgagcagtcatggtataacctgggtgcgacaggcccctggacaagggcttgagtggatgggatggatcagcgctcacaatggtcacgctagcaatgcacagaaggtggaggacagagtcactatgactactgacacatccacgaacacagcctacatggaactgaggagcctgacagctgacgacacggccgtgtattactgtgcgagagtacatgctgccctctactatggtatggacgtctggggccaaggaaccctggtcaccgtctcctcaggtggcggcggttccggaggtggtggtgctggcggtggtggcagctcctatgagctgactcagccaccctcggtgtcactggccccaggacagtcggccaggatttcctgtgggggagacaacattggaagtaaaggtgtacattggtaccagcaaaagccaggccaggcccctgtggtggtcgtctatgatgatcgcgaccggccctcagggatccctgagcgattctctggctccaactctgggaacacggccaccctgaccatcagcagggtcgaagccggggatgaggccgactattactgtcaggtgtgggatagtggtagtgaccactgggttttcggcggagggaccaagctgaccgtcctaggatccggaaaggggcgcgcccatcatcatcatcatcat

SEQ ID NO: 232 - protein, 6 aa, Artificial Sequence
  Description of Artificial Sequence Synthetic 6xHis tag
HHHHHH

SEQ ID NO: 233 - protein, 375 aa, Artificial Sequence
  Description of Artificial Sequence Synthetic polypeptide
GQPKAAPSVTLFPPSSEELQANKATLVCLISDFYPGAVTVAWKADGSPVKAGVETTTPSKQSNNKYAASSYLSLTPEQWKSHRSYSCQVTHEGSTVEKTVAPTECSGGGGSGGGGSSMAQVQLVQSGGEVKKPGASVKVSCKASGYTLSSHGITWVRQAPGQGLEWMGWISAHNGHASNAQKVEDRVTMTTDTSTNTAYMELRSLTADDTAVYYCARVHAALYYGMDVWGQGTLVTVSSGGGGSGGGGAGGGGSSYELTQPPSVSLAPGQSARISCGGDNIGSKGVHWYQQKPGQAPVVVVYDDRDRPSGIPERFSGSNSGNTATLTISRVEAGDEADYYCQVWDSGSDHWVFGGGTKLTVLGSGKGRAHHHHHH

SEQ ID NO: 234 - DNA, 1125 bp, Artificial Sequence
  Description of Artificial Sequence Synthetic polynucleotide
ggtcagcccaaggctgccccctcggtcactctgttcccgccctcctctgaggagcttcaagccaacaaggccacactggtgtgtctcataagtgacttctacccgggagccgtgacagtggcctggaaggcagatggcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcaggtggcggcggttccggaggtggtggttcatcgatggcccaggtgcagctggtgcagtctggaggtgaggtgaagaagccgggggcctcagtgaaggtctcctgcaaggcttctggttacaccttgagcagtcatggtataacctgggtgcgacaggcccctggacaagggcttgagtggatgggatggatcagcgctcacaatggtcacgctagcaatgcacagaaggtggaggacagagtcactatgactactgacacatccacgaacacagcctacatggaactgaggagcctgacagctgacgacacggccgtgtattactgtgcgagagtacatgctgccctctactatggtatggacgtctggggccaaggaaccctggtcaccgtctcctcaggtggcggcggttccggaggtggtggttctggcggtggtggcagcctgcctgtgctgactcagccaccctcagtgtccgcggccccgggacagacggccaggatttcctgtgggggaagcaacattggagataaaggtgtccactggtaccagcagaagccaggccaggcccctgtgctggtcatctatgatgatagcgaccggccctcagggatccctgagcgattctctggctccaactctgggaacacggccaccctgaccatcagcagggtcgaagccggggatgaggccgactattactgtcaggtgtgggatagtagtagtgatcattgggtgttcggcggagggaccaagctgaccgtcctaggatccggaaaggggcgcgcccatcatcatcatcatcat

SEQ ID NO: 235 - protein, 375 aa, Artificial Sequence
  Description of Artificial Sequence Synthetic polypeptide
GQPKAAPSVTLFPPSSEELQANKATLVCLISDFYPGAVTVAWKADGSPVKAGVETTTPSKQSNNKYAASSYLSLTPEQWKSHRSYSCQVTHEGSTVEKTVAPTECSGGGGSGGGGSSMAQVQLVQSGGEVKKPGASVKVSCKASGYTLSSHGITWVRQAPGQGLEWMGWISAHNGHASNAQKVEDRVTMTTDTSTNTAYMELRSLTADDTAVYYCARVHAALYYGMDVWGQGTLVTVSSGGGGSGGGGSGGGGSLPVLTQPPSVSAAPGQTARISCGGSNIGDKGVHWYQQKPGQAPVLVIYDDSDRPSGIPERFSGSNSGNTATLTISRVEAGDEADYYCQVWDSSSDHWVFGGGTKLTVLGSGKGRAHHHHHH

SEQ ID NO: 236 - DNA, 1122 bp, Artificial Sequence
  Description of Artificial Sequence Synthetic polynucleotide
ggtcagcccaaggctgccccctcggtcactctgttcccgccctcctctgaggagcttcaagccaacaaggccacactggtgtgtctcataagtgacttctacccgggagccgtgacagtggcctggaaggcagatggcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcaggtggcggcggttccggaggtggtggttcatcgatggcccaggtgcagctggtgcagtctggaggtgaggtgaagaagccgggggcctcagtgaaggtctcctgcaaggcttctggttacaccttgagcagtcatggtataacctgggtgcgacaggcccctggacaagggcttgagtggatgggatggatcagcgctcacaatggtcacgctagcaatgcacagaaggtggaggacagagtcactatgactactgacacatccacgaacacagcctacatggaactgaggagcctgacagctgacgacacggccgtgtattactgtgcgagagtacatgctgccctctactatggtatggacgtctggggccaaggaaccctggtcaccgtctcctcaggtggcggcggttccggaggtggtggttctggcggtggtggcagctcctatgagctgactcagccaccctcggtgtcagtggccccaggacagacggccaggattacctgtgggggaaacaacattggcaataaaggtgtacactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcattgggtgttcggcggagggaccaagctgaccgtcctaggatccggaaaggggcgcgcccatcatcatcatcat

SEQ ID NO: 237 - protein, 375 aa, Artificial Sequence
  Description of Artificial Sequence Synthetic polypeptide
GQPKAAPSVTLFPPSSEELQANKATLVCLISDFYPGAVTVAWKADGSPVKAGVETTTPSKQSNNKYAASSYLSLTPEQWKSHRSYSCQVTHEGSTVEKTVAPTECSGGGGSGGGGSSMAQVQLVQSGGEVKKPGASVKVSCKASGYTLSSHGITWVRQAPGQGLEWMGWISAHNGHASNAQKVEDRVTMTTDTSTNTAYMELRSLTADDTAVYYCARVHAALYYGMDVWGQGTLVTVSSGGGGSGGGGSGGGGSSYELTQPPSVSVAPGQTARITCGGNNIGNKGVHWYQQKPGQAPVLVVYDDSDRPSGIPERFSGSNSGNTATLTISRVEAGDEADYYCQVWDSSSDHWVFGGGTKLTVLGSGKGRAHHHHHH

SEQ ID NO: 238 - DNA, 1125 bp, Artificial Sequence
  Description of Artificial Sequence Synthetic polynucleotide
ggtcagcccaaggctgccccctcggtcactctgttcccgccctcctctgaggagcttcaagccaacaaggccacactggtgtgtctcataagtgacttctacccgggagccgtgacagtggcctggaaggcagatggcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcaggtggcggcggttccggaggtggtggttcatcgatggcccaggtgcagctggtgcagtctggaggtgaggtgaagaagccgggggcctcagtgaaggtctcctgcaaggcttctggttacaccttgagcagtcatggtataacctgggtgcgacaggcccctggacaagggcttgagtggatgggatggatcagcgctcacaatggtcacgctagcaatgcacagaaggtggaggacagagtcactatgactactgacacatccacgaacacagcctacatggaactgaggagcctgacagctgacgacacggccgtgtattactgtgcgagagtacatgctgccctctactatggtatggacgtctggggccaaggaaccctggtcaccgtctcctcaggtggcggcggttccggaggtggtggttctggcggtggtggcagcctgcctgtgctgactcagccaccctcggtgtcagtggccctaggacagacggccaggattacctgtaggggaaacaacattggtggtaaaggtgtgcactggtaccagcagaagccaggccaggcccctgtgctggtcgtctatgatgattactcccggcgctcaggaatccctgagcgattctctggctcccactctgggagcgcggccaccctgaccatcagcagggtcgaggccggggatgaggccgactattactgtcaggtgtgggatagtagtagtgatcattgggtgttcggcggagggaccaagctgaccgtcctaggatccggaaaggggcgcgcccatcatcatcatcatcat

SEQ ID NO: 239 - protein, 375 aa, Artificial Sequence
  Description of Artificial Sequence Synthetic polypeptide
GQPKAAPSVTLFPPSSEELQANKATLVCLISDFYPGAVTVAWKADGSPVKAGVETTTPSKQSNNKYAASSYLSLTPEQWKSHRSYSCQVTHEGSTVEKTVAPTECSGGGGSGGGGSSMAQVQLVQSGGEVKKPGASVKVSCKASGYTLSSHGITWVRQAPGQGLEWMGWISAHNGHASNAQKVEDRVTMTTDTSTNTAYMELRSLTADDTAVYYCARVHAALYYGMDVWGQGTLVTVSSGGGGSGGGGSGGGGSLPVLTQPPSVSVALGQTARITCRGNNIGGKGVHWYQQKPGQAPVLVVYDDYSRRSGIPERFSGSHSGSAATLTISRVEAGDEADYYCQVWDSSSDHWVFGGGTKLTVLGSGKGRAHHHHHH

SEQ ID NO: 240 - protein, 10 aa, Artificial Sequence
  Description of Artificial Sequence Synthetic peptide
GGGGSGGGGS

SEQ ID NO: 241 - protein, 15 aa, Artificial Sequence
  Description of Artificial Sequence Synthetic peptide
GGGGSGGGGSGGGGS

SEQ ID NO: 242 - protein, 20 aa, Artificial Sequence
  Description of Artificial Sequence Synthetic peptide
GGGGSGGGGSGGGGSGGGGS

SEQ ID NO: 243 - protein, 25 aa, Artificial Sequence
  Description of Artificial Sequence Synthetic peptide
GGGGSGGGGSGGGGSGGGGSGGGGS

SEQ ID NO: 244 - protein, 30 aa, Artificial Sequence
  Description of Artificial Sequence Synthetic polypeptide
GGGGSGGGGSGGGGSGGGGSGGGGSGGGGS

SEQ ID NO: 245 - protein, 35 aa, Artificial Sequence
  Description of Artificial Sequence Synthetic polypeptide
GGGGSGGGGSGGGGSGGGGSGGGGSGGGGSGGGGS

SEQ ID NO: 246 - protein, 20 aa, Artificial Sequence
  Description of Artificial Sequence Synthetic peptide
  MISC_FEATURE (1)..(20): This sequence may encompass 1-10 “Gly Ser” repeating units
GSGSGSGSGSGSGSGSGSGS

SEQ ID NO: 247 - protein, 30 aa, Artificial Sequence
  Description of Artificial Sequence Synthetic polypeptide
  MISC_FEATURE (1)..(30): This sequence may encompass 1-10 “Gly Gly Ser” repeating units
GGSGGSGGSGGSGGSGGSGGSGGSGGSGGS

SEQ ID NO: 248 - protein, 40 aa, Artificial Sequence
  Description of Artificial Sequence Synthetic polypeptide
  MISC_FEATURE (1)..(40): This sequence may encompass 1-10 “Gly Gly Gly Ser” repeating units
GGGSGGGSGGGSGGGSGGGSGGGSGGGSGGGSGGGSGGGS

SEQ ID NO: 249 - protein, 40 aa, Artificial Sequence
  Description of Artificial Sequence Synthetic polypeptide
  MISC_FEATURE (1)..(40): This sequence may encompass 1-10 “Gly Gly Ser Gly” repeating units
GGSGGGSGGGSGGGSGGGSGGGSGGGSGGGSGGGSGGGSG

SEQ ID NO: 250 - protein, 50 aa, Artificial Sequence
  Description of Artificial Sequence Synthetic polypeptide
  MISC_FEATURE (1)..(50): This sequence may encompass 1-10 “Gly Gly Ser Gly Gly” repeating units
GGSGGGGSGGGGSGGGGSGGGGSGGGGSGGGGSGGGGSGGGGSGGGGSGG

SEQ ID NO: 251 - protein, 50 aa, Artificial Sequence
  Description of Artificial Sequence Synthetic polypeptide
  MISC_FEATURE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS

SEQ ID NO: 252 - protein, 98 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 253 - protein, 120 aa, Artificial Sequence
  Description of Artificial Sequence Synthetic polypeptide
EVQLVQSGGEVKKPGASVKVSCKASGYTLSSHGVTWVRQAPGQGLEWMGWISAHNGHASNAQKVEDRVTMTTDTSTNTAYMELRSLTPDDTAVYYCARVHAALYYGMDVWGQGTMVTVSS

SEQ ID NO: 254 - protein, 98 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 255 - protein, 98 aa, Homo sapiens
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAR

SEQ ID NO: 256 - protein, 98 aa, Homo sapiens
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAR

SEQ ID NO: 257 - protein, 100 aa, Homo sapiens
EVQLVESGGGLVKPGGSLRLSCAASGFTFSNAWMNWVRQAPGKGLEWVGRIKSKTDGGTTDYAAPVKGRFTISRDDSKNTLYLQMNSLKTEDTAVYYCTT

SEQ ID NO: 258 - protein, 98 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 259 - protein, 98 aa, Homo sapiens
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCAR

SEQ ID NO: 260 - protein, 4 aa, Artificial Sequence
  Description of Artificial Sequence Synthetic peptide
CPSC

SEQ ID NO: 261 - protein, 4 aa, Artificial Sequence
  Description of Artificial Sequence Synthetic peptide
CPPC

SEQ ID NO: 262 - protein, 5 aa, Artificial Sequence
  Description of Artificial Sequence Synthetic peptide
GGGGS

(19) United States
(12) Patent Application Publication
(10) Pub. No.: US 2023/0127123 A1
(43) Pub. Date: Apr. 27, 2023

(54) ANTIBODIES AGAINST PD-L1 AND METHODS OF USE THEREOF

(71) Applicant: Dana-Farber Cancer Institute, Inc., Boston, MA (US)

(72) Inventors: Wayne A. Marasco, Wellesley, MA (US); Matthew Chang, Brookline, MA (US)

(21) Appl. No.: 17/780,389

(22) PCT Filed: Dec. 2, 2020
(86) PCT No.: PCT/US2020/062815

§ 371 Date: May 26, 2022

Related U.S. Application Data
(60) Provisional application No. 62/942,455, filed on Dec. 2, 2019.

(51) Int. Cl. C07K 16/28; A61K 39/395; A61K 45/06; C07K 14/705; C07K 14/725

(52) U.S. Cl. CPC C07K 16/2827; A61K 39/3955; A61K 45/06; C07K 14/70521; C07K 14/70578; C07K 14/7051; C07K 2317/565; C07K 2317/31; C07K 2319/03; C07K 2319/02; C07K 2319/33; C07K 2317/55; C07K 2317/622; C07K 2317/92

(57) ABSTRACT

The present invention is directed to human monoclonal antibodies that bind to the cell-surface receptor, PDL-1 (programmed death ligand 1). The antibodies can be used to treat cancer and chronic viral infections.

This application is a National Stage Entry of PCT/US2020/062815, filed Dec. 02, 2020, which claims the benefit of priority from U.S. provisional patent application no. 62/942,455, filed on Dec. 2, 2019, the contents of each which are incorporated herein by reference in their entireties.

### GOVERNMENT INTERESTS

This invention was made with government support under Grant No. 1 R56 AI109223-01A1 awarded by the National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### SEQUENCE LISTING

The instant application contains a Sequence Listing which has been submitted electronically in ASCII format and is hereby incorporated by reference in its entirety. Said ASCII copy, created on Jan. 13, 2021, is named 5031461-045WO1_SL.txt and is 149,240 bytes in size.

### FIELD OF THE INVENTION

This invention is directed to antibodies against PD-L1 (also known as programmed cell death 1 ligand 1, or B7H1) and methods of use thereof.

### BACKGROUND OF THE INVENTION

Programmed cell death-1 (PD-1), is a cell surface membrane protein of the immunoglobulin superfamily. This protein is expressed in pro-B-cells and is thought to play a role in their differentiation. A member of the CD28 family, PD-1 is upregulated on activated T cells, B cells, and monocytes. PD-1 has two identified ligands in the B7 family, PD-L1 (programmed cell death-1 ligand 1; also known as cluster of differentiation 274 (CD274) or B7 homolog 1 (B7-H1)) and PD-L2. PD-L1 is a 40 kDa type I transmembrane protein. The binding of PD-L1 to PD-1 or B7.1 transmits an inhibitory signal which reduces the proliferation of CD8+ T cells at the lymph nodes. While PD-L2 expression tends to be more restricted, found primarily on activated antigen-presenting cells (APCs), PD-L1 expression is more widespread, including cells of hematopoietic lineage (including activated T cells, B cells, monocytes, dendritic cells and macrophages) and peripheral nonlymphoid tissues (including heart, skeletal, muscle, placenta, lung, kidney and liver tissues). The widespread expression of PD-L1 indicates its significant role in regulating PD-1/PD-L1-mediated peripheral tolerance.

### SUMMARY OF THE INVENTION

An aspect of the invention is directed to isolated monoclonal antibodies that bind to human Programmed death-ligand 1 (PD-L1). In some embodiments, the antibody can be an antigen-binding fragment thereof that binds to human Programmed death-ligand 1 (PD-L1). In other embodiments, the antibody or fragment can comprise a heavy chain, light chain, or a combination thereof. In one embodiment, the heavy chain comprises a CDR1 comprising G-(X₁)-T-(X₂)-SS-(X₃X₄) (SEQ ID NO: 47), G-(X₁)-T-(X₂)-(X₁₃ X₁₄)-(X₃X₄) (SEQ ID NO: 205), G-(X₁)-TF-(X₁₃X₁₄)-Y-(X₄) (SEQ ID NO: 206), CDR2 comprising I-(X₈X₉X₁₀ X₁₁-G- (X₁₂)-A (SEQ ID NO: 51) or II-(X₁₅)-IFG-(X₁₆)-A (SEQ ID NO: 207), and/or a CDR3 comprising ARGRQMFGAGIDF (SEQ ID NO: 6), ARVHAALYYGMDV (SEQ ID NO: 14), TTGGLGLVYPYYNYIDV (SEQ ID NO: 99), AKVHPVFSYALDV (SEQ ID NO: 100), AEEGAFNSLAI (SEQ ID NO: 101), ARDGSGYDSAGMDD (SEQ ID NO: 102), ARGFGGPDY (SEQ ID NO: 103), ARVHGALYYGMDV (SEQ ID NO: 104), ASGSIVGAAYAFDI (SEQ ID NO: 105), ARDRSEGGFDP (SEQ ID NO: 106), or AEEGAFNSLAI (SEQ ID NO: 107). In another embodiment, the light chain comprises a CDR1 comprising SGSIDSNY (SEQ ID NO: 18), S-(X₁₇X₁₈)I-(X₁₉)-SNY (SEQ ID NO: 208), or NIG-(X₅)-K-(X₂₀) (SEQ ID NO: 48), a CDR2 comprising EDN (SEQ ID NO: 20), (X₂₁)-DN (SEQ ID NO: 209), (X₂₂)-NN (SEQ ID NO: 210), or DD-X₆ (SEQ ID NO: 49), and/or a CDR3 comprising QSYDSNNRHVI (SEQ ID NO: 22), QVWDS-(X₇)-SDHWV (SEQ ID NO: 50), QVWDSSGDLWV (SEQ ID NO: 126), AAWDDSLNGLV (SEQ ID NO: 127), QSYDGITVI (SEQ ID NO: 128), QSYDSSNHWV (SEQ ID NO: 129), AVWDDSLSGVV (SEQ ID NO: 131), MIWHSSAYV (SEQ ID NO: 132), NSRDISDNQWQWI (SEQ ID NO: 134), or QSYDSSNHVV (SEQ ID NO: 135). In some embodiments, the antibody is fully human or humanized. In other embodiments, the antibody is monospecific, bispecific, or multispecific. In further embodiments, the antibody is a single chain antibody. In some embodiments, the antibody has a binding affinity of at least 3.3×10-9 M . In embodiments, the antibody or fragment can further comprise a heavy chain constant region, a light chain constant region, an Fc region , or a combination thereof. In embodiments, X₁, X₂, X₃, or X₄ is a non-polar amino acid residue. In other embodiments, X₁, X₂, X₃, or X₄ is glycine (G), tyrosine (Y), phenylalanine (F), leucine (L), or alanine (A). In some embodiments, X₁, X₂, or X₄ is a hydrophobic amino acid residue, for example X₁, X₂, or X₄ is glycine (G), leucine (L), or alanine (A). In some embodiments, X₃ is a hydrophilic polar amino acid residue. In one embodiment, X₃ is histidine (H). In some embodiments, X₁ is phenylalanine (F), glycine (G) or tyrosine (Y). In further embodiments, X₂ is phenylalanine (F) or leucine (L). In other embodiments, X₃ is histidine (H) or tyrosine (Y). In yet further embodiments, X₄ is serine (S), glycine (G) or alanine (A). In one embodiment, X₈, X₉, X₁₀, or X₁₁ is a non-polar hydrophobic amino acid residue. In some embodiments, X₈, X₉, X₁₀, or X₁₁ is isoleucine (I), proline (P), alanine (A), or phenylalanine (F). In other embodiments, X₈, X₁₀, or X₁₂ is a polar hydrophilic amino acid residue. In yet further embodiments, X₈, X₁₀, or X₁₂ is histidine (H), serine (S), asparagine (N), or threonine (T). In one embodiment, X₈ is alanine (A), isoleucine (I) or serine (S). In one embodiment, X₉ tyrosine (Y), serine (S), proline (P) or alanine (A). In one embodiment, X₁₀ is tyrosine (Y), aspartate (D), isoleucine (I) or histidine (H). In one embodiment, X₁₁ is glycine (G), leucine (L), asparagine (N) or phenylalanine (F). In one embodiment, X₁₂ is isoleucine (I), arginine (R), threonine (T) or histidine (H). In some embodiments, X₅ is a non-polar hydrophobic amino acid residue. In one embodiment, X₅ is glycine (G). In other embodiments, X₅ is a polar hydrophilic amino acid residue. In one embodiment, X₅ is serine (S), asparagine (N), or aspartate (D). In further embodiments, X₆ is a non-polar amino acid residue. In one embodiment, X₆ is tyrosine (Y). In some embodiments, X₆ is a polar hydrophilic amino acid residue. In one embodiment, X₆ is threonine (T), serine (S) or arginine (R). In some embodiments, X₇ X₁₅, X₁₆, X₁₇, X₁₉, X_{20,} or X₂₁ is a non-polar hydrophobic amino acid residue. In one embodiment, X₇, X₁₇, or X₂₀ is glycine (G). In other embodiments, X₇, X₁₃, X₁₄, X₁₅, X₁₆, X₁₇, X₁₈, X₁₉, or X₂₁ is a polar hydrophilic amino acid residue. In one embodiment, X₇ X₁₄, or X₂₁ is serine (S) or arginine (R). In one embodiment, X₁₃ is serine (S) or threonine (T). In one embodiment, X₁₅ is proline (P). In one embodiment, X₁₅, X₁₇, or X₂₀ is serine (S). In one embodiment, X₁₆ is threonine (T) or arginine (R). In one embodiment, X₁₆ isoleucine (I). In one embodiment, X₁₈ is serine (S) or asparagine (N). In one embodiment, X₁₉ is glycine (G), or alanine (A). In one embodiment, X₁₉ is aspartate (D). In one embodiment, X₂₁ is alanine (A). In one embodiment, X₂₁ is glutamate (E).

An aspect of the invention is directed to an isolated antibody or fragment thereof that binds to human Programmed death-ligand 1 (PD-L1) protein and comprises (a) a VH CDR1 comprising the amino acid sequence of SEQ ID NO: 2, a VH CDR2 comprising the amino acid sequence of SEQ ID NO: 4, a VH CDR3 comprising the amino acid sequence of SEQ ID NO: 6, a VL CDR1 comprising the amino acid sequence of SEQ ID NO: 18, a VL CDR2 comprising the amino acid sequence of SEQ ID NO: 20, and a VL CDR3 comprising the amino acid sequence of SEQ ID NO: 22; or (b) a VH CDR1 comprising the amino acid sequence of SEQ ID NO: 10, a VH CDR2 comprising the amino acid sequence of SEQ ID NO: 12, a VH CDR3 comprising the amino acid sequence of SEQ ID NO: 14, a VL CDR1 comprising the amino acid sequence of SEQ ID NO: 48, a VL CDR2 comprising the amino acid sequence of SEQ ID NO: 49, and a VL CDR3 comprising the amino acid sequence of SEQ ID NO: 50. In some embodiments, X₅ of SEQ ID NO: 48 is a non-polar hydrophobic amino acid residue. In one embodiment, X₅ is glycine (G). In other embodiments, X₅ is a polar hydrophilic amino acid residue. In one embodiment, X₅ is serine (S), asparagine (N), or aspartate (D). In further embodiments, X₆ of SEQ ID NO: 49 is a non-polar amino acid residue. In one embodiment, X₆ is tyrosine (Y). In some embodiments, X₆ is a polar hydrophilic amino acid residue. In one embodiment, X₆ is serine (S), threonine (T), or arginine (R). In some embodiments, X₇ of SEQ ID NO: 50 is a non-polar hydrophobic amino acid residue. In one embodiment, X₇ is glycine (G). In other embodiments, X₇ is a polar hydrophilic amino acid residue. In one embodiment, X₇ is serine (S) or arginine (R). In some embodiments, VL CDR1 comprises the amino acid sequence of SEQ ID NOS: 26, 33, 40, or 44. In embodiments, VL CDR2 comprises the amino acid sequence of SEQ ID NOS: 28, 35, or 45. In embodiments, VL CDR3 comprises the amino acid sequence of SEQ ID NOS: 30, or 37. In some embodiments, the antibody of (b) described herein comprises a VL CDR1 comprising the amino acid sequence of SEQ ID NO: 26, a VL CDR2 comprising the amino acid sequence of SEQ ID NO: 28, and a VL CDR3 comprising the amino acid sequence of SEQ ID NO: 30; or comprises a VL CDR1 comprising the amino acid sequence of SEQ ID NO: 33, a VL CDR2 comprising the amino acid sequence of SEQ ID NO: 35, and a VL CDR3 comprising the amino acid sequence of SEQ ID NO: 37; or comprises a VL CDR1 comprising the amino acid sequence of SEQ ID NO: 40, a VL CDR2 comprising the amino acid sequence of SEQ ID NO: 35, and a VL CDR3 comprising the amino acid sequence of SEQ ID NO: 37; or comprises a VL CDR1 comprising the amino acid sequence of SEQ ID NO: 44, a VL CDR2 comprising the amino acid sequence of SEQ ID NO: 45, and a VL CDR3 comprising the amino acid sequence of SEQ ID NO: 37.

An aspect of the invention is directed to an isolated antibody or fragment thereof that binds to human PD-L1 protein and comprises a heavy chain variable region comprising an amino acid sequence selected from the group consisting of SEQ ID NO: 16, 52, 54, 56, 60, 62, 64, 66, 68, 70, 72, 74, 76, 78, 80, and 82, and a light chain variable region comprising an amino acid sequence selected from the group consisting of SEQ ID NOS: 24, 31, 38, 42, 46, 53, 55, 57, 59, 61, 63, 65, 67, 69, 71, 73, 75, 77, 79, 81, and 83.

An aspect of the invention is directed to an isolated antibody or fragment thereof that binds to human PD-L1 protein and comprises a heavy chain variable region comprising: a VH-CDR1 having an amino acid sequence selected from the group consisting of SEQ ID NOS: 2, 10, 84, 85, 86, 87, 88, 89, and 90; a VH-CDR2 having an amino acid sequence selected from the group consisting of SEQ ID NOS: 4, 12, 91, 92, 93, 94, 95, 96, 97, and 98: and/or a VH-CDR3 having an amino acid sequence selected from the group consisting of SEQ ID NOS: 6, 14, 99, 100, 101, 102, 103, 104, 105, 106, and 107; and/or a light chain variable region comprising: a VL-CDR1 having an amino acid sequence selected from the group consisting of SEQ ID NOS: 18, 26, 33, 40, 44, 108, 109, 110, 111, 112, 113, 114, 115, 116, and 117; a VL-CDR2 having an amino acid sequence selected from the group consisting of SEQ ID NOS: 20, 28, 35, 45, 118, 119, 120, 121, 122, 123, 124, and 125; and/or a VL-CDR3 having an amino acid sequence selected from the group consisting of SEQ ID NOS: 22, 30, 37, 126, 127, 128, 129, 130, 131, 132, 133, 134, and 135.

An aspect of the invention is directed to an isolated antibody or fragment thereof that binds to human PD-L1 protein and wherein the antibody comprises: (a) a heavy chain variable region with three CDRs comprising the amino acid sequences GGTFSSYA (SEQ ID NO: 2), IIPIFGTA (SEQ ID NO: 4), and ARGRQMFGAGIDF (SEQ ID NO: 6) respectively, and/or a light chain variable region with three CDRs comprising the amino acid sequences SGSIDSNY (SEQ ID NO:18), EDN (SEQ ID NO:20), and QSYDSNNRHVI (SEQ ID NO:22) respectively; (b) a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGSKG (SEQ ID NO:26), DDR (SEQ ID NO:28), and QVWDSGSDHWV (SEQ ID NO:30) respectively; (c) a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGDKG (SEQ ID NO:33), DDS (SEQ ID NO:35), and QVWDSSSDHWV (SEQ ID NO:37) respectively; (d) a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGNKG (SEQ ID NO:40), DDS (SEQ ID NO:35), and QVWDSSSDHWV (SEQ ID NO:37) respectively; (e) a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGGKG (SEQ ID NO:44), DDY (SEQ ID NO:45), and QVWDSSSDHWV (SEQ ID NO:37) respectively; (0 a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIESRS (SEQ ID NO: 108), DDT (SEQ ID NO: 118), and QVWDSSGDLWV (SEQ ID NO: 126) respectively; (g) a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGSKG (SEQ ID NO: 26), DDS (SEQ ID NO:35), and QVWDSSSDHWV (SEQ ID NO:37) respectively; (h) a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGSKS (SEQ ID NO: 109), DDS (SEQ ID NO:35), and QVWDSSSDHWV (SEQ ID NO:37); (i) a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGSKG (SEQ ID NO: 26), DDS (SEQ ID NO:35), and QVWDSSSDHWV (SEQ ID NO:37) respectively; (j) a heavy chain variable region with three CDRs comprising the amino acid sequences DFAFSSAW (SEQ ID NO: 84), IKSKTDGETT (SEQ ID NO: 91), and TTGGLGLVYPYYNYIDV (SEQ ID NO: 99) respectively, and/or a light chain variable region with three CDRs comprising the amino acid sequences SSNIGSNY (SEQ ID NO: 110), RNN (SEQ ID NO: 119), and AAWDDSLNGLV (SEQ ID NO: 127) respectively; (k) a heavy chain variable region with three CDRs comprising the amino acid sequences GYTFTSYG (SEQ ID NO: 85), TSPHNGLT (SEQ ID NO: 92), and AKVHPVFSYALDV (SEQ ID NO: 100) respectively, and/or a light chain variable region with three CDRs comprising the amino acid sequences SGSIASNY (SEQ ID NO: 111), EDN (SEQ ID NO: 20), and QSYDGITVI (SEQ ID NO: 128) respectively; (1) a heavy chain variable region with three CDRs comprising the amino acid sequences GGTFSRYA (SEQ ID NO: 86), IIPIFGRA (SEQ ID NO: 93), and AEEGAFNSLAI (SEQ ID NO: 101) respectively, and/or a light chain variable region with three CDRs comprising the amino acid sequences SGSIASNY (SEQ ID NO: 111), ADN (SEQ ID NO: 120), and QSYDSSNHWV (SEQ ID NO: 129) respectively; (m) a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGSKS (SEQ ID NO: 109), DDS (SEQ ID NO:35), and QVWDSSSDHWV (SEQ ID NO:37) respectively; (n) a heavy chain variable region with three CDRs comprising the amino acid sequences GYTFTSYG (SEQ ID NO: 85), ISAYNGHA (SEQ ID NO: 94), and ARVHAALYYGMDV (SEQ ID NO: 14) respectively, and/or a light chain variable region with three CDRs comprising the amino acid sequences NIGSKG (SEQ ID NO: 26), DDS (SEQ ID NO: 35), and QVWDSRSDHWV (SEQ ID NO: 130) respectively; (o) a heavy chain variable region with three CDRs comprising the amino acid sequences GGTFSSYA (SEQ ID NO: 87), IIPIFGTA (SEQ ID NO: 95), and ARDGSGYDSAGMDD (SEQ ID NO: 102) respectively, and/or a light chain variable region with three CDRs comprising the amino acid sequences RSNIGSNY (SEQ ID NO: 112), SNN (SEQ ID NO: 121), and AVWDDSLSGVV (SEQ ID NO: 131) respectively; (p) a heavy chain variable region with three CDRs comprising the amino acid sequences GFTFSSYA (SEQ ID NO: 88), ISYDGSNK (SEQ ID NO: 96), and ARGFGGPDY (SEQ ID NO: 103) respectively, and/or a light chain variable region with three CDRs comprising the amino acid sequences SGINVGTYR (SEQ ID NO: 113), YKSDSDK (SEQ ID NO: 122), and MIWHSSAYV (SEQ ID NO: 132) respectively; (q) a heavy chain variable region with three CDRs comprising the amino acid sequences GYTFSSYG (SEQ ID NO: 89), ISAHNGHA (SEQ ID NO: 12), and ARVHGALYYGMDV (SEQ ID NO: 104) respectively, and/or a light chain variable region with three CDRs comprising the amino acid sequences NIGGKS (SEQ ID NO: 114), DDR (SEQ ID NO: 28), and QVWDSSSDHWV (SEQ ID NO: 37) respectively; (r) a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGSKG (SEQ ID NO: 26), DDR (SEQ ID NO: 28), and QVWDSSSDHWV (SEQ ID NO: 37) respectively; (s) a heavy chain variable region with three CDRs comprising the amino acid sequences GGTFSSYA (SEQ ID NO: 87), IIPILGIA (SEQ ID NO: 97), and ASGSIVGAAYAFDI (SEQ ID NO: 105) respectively, and/or a light chain variable region with three CDRs comprising the amino acid sequences NIGGRV (SEQ ID NO: 115), DDT (SEQ ID NO: 123), and QVWDSRSDHPV (SEQ ID NO: 133) respectively; (t) a heavy chain variable region with three CDRs comprising the amino acid sequences GFTFSSYS (SEQ ID NO: 90), IISDGSAT (SEQ ID NO: 98), and ARDRSEGGFDP (SEQ ID NO: 106) respectively, and/or a light chain variable region with three CDRs comprising the amino acid sequences SLRSYY (SEQ ID NO: 116), GKN (SEQ ID NO: 124), and NSRDISDNQWQWI (SEQ ID NO: 134) respectively; or (u) a heavy chain variable region with three CDRs comprising the amino acid sequences GGTFSRYA (SEQ ID NO: 86), IIPIFGRA (SEQ ID NO: 93), and AEEGAFNSLAI (SEQ ID NO: 107) respectively, and/or a light chain variable region with three CDRs comprising the amino acid sequences SGSIASHF (SEQ ID NO: 117), GDD (SEQ ID NO: 125), and QSYDSSNHVV (SEQ ID NO: 135) respectively.

An aspect of the invention is directed to an isolated monoclonal antibody or antigen-binding fragment thereof that binds to PD-L1 comprising a heavy chain, a light chain, or a combination thereof, wherein the heavy chain comprises an amino acid sequence about 95% identical to SEQ ID NO: 8, and the light chain comprises an amino acid sequence about 95% identical to SEQ ID NO: 24.

An aspect of the invention is directed to an isolated monoclonal antibody or antigen-binding fragment thereof that binds to PD-L1 comprising a heavy chain, a light chain, or a combination thereof, wherein the heavy chain comprises an amino acid sequence about 95% identical to SEQ ID NO: 16, and the light chain comprises an amino acid sequence about 95% identical to SEQ ID NO: 31.

An aspect of the invention is directed to an isolated monoclonal antibody or antigen-binding fragment thereof that binds to PD-L1 comprising a heavy chain, a light chain, or a combination thereof, wherein the heavy chain comprises an amino acid sequence about 95% identical to SEQ ID NO: 16, and the light chain comprises an amino acid sequence about 95% identical to SEQ ID NO: 38.

An aspect of the invention is directed to an isolated monoclonal antibody or antigen-binding fragment thereof that binds to PD-L1 comprising a heavy chain, a light chain, or a combination thereof, wherein the heavy chain comprises an amino acid sequence about 95% identical to SEQ ID NO: 16, and the light chain comprises an amino acid sequence about 95% identical to SEQ ID NO: 42.

An aspect of the invention is directed to an isolated monoclonal antibody or antigen-binding fragment thereof that binds to PD-L1 comprising a heavy chain, a light chain, or a combination thereof, wherein the heavy chain comprises an amino acid sequence about 95% identical to SEQ ID NO: 16, and the light chain comprises an amino acid sequence about 95% identical to SEQ ID NO: 46.

An aspect of the invention is directed to an isolated monoclonal antibody or antigen-binding fragment thereof that binds to PD-L1 comprising a heavy chain, a light chain, or a combination thereof, wherein:
(a) the heavy chain comprises an amino acid sequence about 95% identical to SEQ ID NO: 52, and the light chain comprises an amino acid sequence about 95% identical to SEQ ID NO: 53;
(b) the heavy chain comprises an amino acid sequence about 95% identical to SEQ ID NO: 54, and the light chain comprises an amino acid sequence about 95% identical to SEQ ID NO: 55;
(c) the heavy chain comprises an amino acid sequence about 95% identical to SEQ ID NO: 56, and the light chain comprises an amino acid sequence about 95% identical to SEQ ID NO: 57;
(d) the heavy chain comprises an amino acid sequence about 95% identical to SEQ ID NO: 16, and the light chain comprises an amino acid sequence about 95% identical to SEQ ID NO: 59;
(e) the heavy chain comprises an amino acid sequence about 95% identical to SEQ ID NO: 60, and the light chain comprises an amino acid sequence about 95% identical to SEQ ID NO: 61;
(f) the heavy chain comprises an amino acid sequence about 95% identical to SEQ ID NO: 62, and the light chain comprises an amino acid sequence about 95% identical to SEQ ID NO: 63;
(g) the heavy chain comprises an amino acid sequence about 95% identical to SEQ ID NO: 64, and the light chain comprises an amino acid sequence about 95% identical to SEQ ID NO: 65;
(h) the heavy chain comprises an amino acid sequence about 95% identical to SEQ ID NO: 66, and the light chain comprises an amino acid sequence about 95% identical to SEQ ID NO: 67;
(i) the heavy chain comprises an amino acid sequence about 95% identical to SEQ ID NO: 68, and the light chain comprises an amino acid sequence about 95% identical to SEQ ID NO: 69;
(j) the heavy chain comprises an amino acid sequence about 95% identical to SEQ ID NO: 70, and the light chain comprises an amino acid sequence about 95% identical to SEQ ID NO: 71;
(k) the heavy chain comprises an amino acid sequence about 95% identical to SEQ ID NO: 72, and the light chain comprises an amino acid sequence about 95% identical to SEQ ID NO: 73;
(l) the heavy chain comprises an amino acid sequence about 95% identical to SEQ ID NO: 74, and the light chain comprises an amino acid sequence about 95% identical to SEQ ID NO: 75;
(m)the heavy chain comprises an amino acid sequence about 95% identical to SEQ ID NO: 76, and the light chain comprises an amino acid sequence about 95% identical to SEQ ID NO: 77;
(n) the heavy chain comprises an amino acid sequence about 95% identical to SEQ ID NO: 78, and the light chain comprises an amino acid sequence about 95% identical to SEQ ID NO: 79;
(o) the heavy chain comprises an amino acid sequence about 95% identical to SEQ ID NO: 80, and the light chain comprises an amino acid sequence about 95% identical to SEQ ID NO: 81; or
(p) the heavy chain comprises an amino acid sequence about 95% identical to SEQ ID NO: 82; and the light chain comprises an amino acid sequence about 95% identical to SEQ ID NO: 83.

An aspect of the invention is directed to an isolated monoclonal antibody or antigen-binding fragment thereof that binds to PD-L1 comprising a heavy chain, a light chain, or a combination thereof, wherein the antibody or fragment comprises:
(a) a V_{H} amino acid sequence having SEQ ID NO: 8, and a V_{L} amino acid sequence having SEQ ID NO: 24;
(b) a V_{H} amino acid sequence having SEQ ID NO: 16, and a V_{L} amino acid sequence having SEQ ID NO: 31;
(c) a V_{H} amino acid sequence having SEQ ID NO: 16, and a V_{L} amino acid sequence having SEQ ID NO: 38;
(d) a V_{H} amino acid sequence having SEQ ID NO: 16, and a V_{L} amino acid sequence having SEQ ID NO: 42;
(e) a V_{H} amino acid sequence having SEQ ID NO: 16, and a V_{L} amino acid sequence having SEQ ID NO: 46;
(f) a V_{H} amino acid sequence having SEQ ID NO: 52, and a V_{L} amino acid sequence having SEQ ID NO: 53;
(g) a VH amino acid sequence having SEQ ID NO: 54, and a V_{L} amino acid sequence having SEQ ID NO: 55;
(h) a V_{H} amino acid sequence having SEQ ID NO: 56, and a V_{L} amino acid sequence having SEQ ID NO: 57;
(i) a V_{H} amino acid sequence having SEQ ID NO: 16, and a V_{L} amino acid sequence having SEQ ID NO: 59;
(j) a V_{H} amino acid sequence having SEQ ID NO: 60, and a V_{L} amino acid sequence having SEQ ID NO: 61;
(k) a VH amino acid sequence having SEQ ID NO: 62, and a V_{L} amino acid sequence having SEQ ID NO: 63;
(l) a V_{H} amino acid sequence having SEQ ID NO: 64, and a V_{L} amino acid sequence having SEQ ID NO: 65;
(m)a V_{H} amino acid sequence having SEQ ID NO: 66, and a V_{L} amino acid sequence having SEQ ID NO: 67;
(n) a VH amino acid sequence having SEQ ID NO: 68, and a V_{L} amino acid sequence having SEQ ID NO: 69;
(o) a V_{H} amino acid sequence having SEQ ID NO: 70, and a V_{L} amino acid sequence having SEQ ID NO: 71;
(p) a V_{H} amino acid sequence having SEQ ID NO: 72, and a V_{L} amino acid sequence having SEQ ID NO: 73;
(q) a VH amino acid sequence having SEQ ID NO: 74, and a V_{L} amino acid sequence having SEQ ID NO: 75;
(r) a V_{H} amino acid sequence having SEQ ID NO: 76, and a V_{L} amino acid sequence having SEQ ID NO: 77;
(s) a V_{H} amino acid sequence having SEQ ID NO: 78, and a V_{L} amino acid sequence having SEQ ID NO: 79;
(t) a V_{H} amino acid sequence having SEQ ID NO: 80, and a V_{L} amino acid sequence having SEQ ID NO: 81; or
(u) a V_{H} amino acid sequence having SEQ ID NO: 82; and a V_{L} amino acid sequence having SEQ ID NO: 83

Aspects of the invention are directed to an isolated bispecific antibody comprising a fragment of the antibodies described herein and a second antigen-binding fragment having specificity to a molecule on an immune cell. In some embodiments, the molecule on an immune cell is selected from the group consisting of B7H3, B7H4, CD27, CD28, CD40, CD40L, CD47, CD122, CTLA-4, GITR, GITRL, ICOS, ICOSL, LAG-3, LIGHT, OX-40, OX40L, PD-1, TIM3, 4-IBB, TIGIT, VISTA, HEVM, BTLA, and MR. In some embodiments, the fragment and the second fragment each is independently selected from a Fab fragment, a single-chain variable fragment (scFv), or a single-domain antibody. In further embodiments, the isolated bispecific antibody further comprises a Fc fragment.

Aspects of the invention are directed to nucleic acids encoding antibodies described herein.

Aspects of the invention are directed to a vector comprising nucleic acids encoding antibodies described herein.

Aspects of the invention are directed to a cell comprising a vector comprising nucleic acids encoding bispecific antibodies described herein

Aspects of the invention are directed to nucleic acids encoding bispecific antibodies described herein.

Aspects of the invention are directed to a vector comprising nucleic acids encoding bispecific antibodies described herein.

Aspects of the invention are directed to a cell comprising a vector comprising nucleic acids encoding bispecific antibodies described herein.

Aspects of the invention are directed to pharmaceutical compositions comprising antibodies described or fragments described herein, and a pharmaceutically acceptable carrier or excipient. In some embodiments, the pharmaceutical composition further comprises at least one additional therapeutic agent. In other embodiments, the therapeutic agent is a toxin, a radiolabel, a siRNA, a small molecule, or a cytokine.

Aspects of the invention are directed to pharmaceutical compositions comprising bispecific antibodies described herein, and a pharmaceutically acceptable carrier or excipient. In some embodiments, the pharmaceutical composition further comprises at least one additional therapeutic agent. In other embodiments, the therapeutic agent is a toxin, a radiolabel, a siRNA, a small molecule, or a cytokine.

Aspects of the invention are directed to an isolated cell comprising one or more polynucleotide(s) encoding the antibody or fragment thereof described herein.

Aspects of the invention are directed to an isolated cell comprising one or more polynucleotide(s) encoding the bispecific antibody described herein.

Aspects of the invention are directed to a kit comprising: the at least one antibody composition described herein; a syringe, needle, or applicator for administration of the at least one antibody to a subject; and instructions for use.

An aspect of the invention is directed to a chimeric antigen receptor (CAR). In some embodiments, the CAR comprises an intracellular signaling domain, a transmembrane domain and an extracellular domain, wherein the extracellular domain is an isolated monoclonal antibody or antigen-binding fragment thereof that binds to human Programmed death-ligand 1 (PD-L1) protein, wherein the monoclonal antibody or fragment thereof comprises a heavy chain, light chain, or combination thereof. In some embodiments, the heavy chain comprises a CDR1 comprising G-(X1)-T-(X2)-SS-(X3X4) (SEQ ID NO: 47), G-(X₁)-T-(X₂)-(X₁₃X₁₄)-(X₃X₄) (SEQ ID NO: 205), G-(X₁)-TF-(X₁₃X₁₄)-Y-(X₄) (SEQ ID NO: 206), CDR2 comprising I-(X₈X₉X₁₀X₁₁)-G-(X₁₂)-A (SEQ ID NO: 51) or II-(X₁₅)-IFG-(X₁₆)-A (SEQ ID NO: 207), and/or a CDR3 comprising ARGRQMFGAGIDF (SEQ ID NO: 6), ARVHAALYYGMDV (SEQ ID NO: 14), TTGGLGLVYPYYNYIDV (SEQ ID NO: 99), AKVHPVFSYALDV (SEQ ID NO: 100), AEEGAFNSLAI (SEQ ID NO: 101), ARDGSGYDSAGMDD (SEQ ID NO: 102), ARGFGGPDY (SEQ ID NO: 103), ARVHGALYYGMDV (SEQ ID NO: 104), ASGSIVGAAYAFDI (SEQ ID NO: 105), ARDRSEGGFDP (SEQ ID NO: 106), or AEEGAFNSLAI (SEQ ID NO: 107). In another embodiment, the light chain comprises a CDR1 comprising SGSIDSNY (SEQ ID NO: 18), S-(X₁₇X₁₈)I-(X₁₉)-SNY (SEQ ID NO: 208), or NIG-(X₅)-K-(X₂₀) (SEQ ID NO: 48), a CDR2 comprising EDN (SEQ ID NO: 20), (X₂₁)-DN (SEQ ID NO: 209), (X₂₂)-NN (SEQ ID NO: 210), or DD-X₆ (SEQ ID NO: 49), and/or a CDR3 comprising QSYDSNNRHVI (SEQ ID NO: 22), QVWDS-(X7)-SDHWV (SEQ ID NO: 50), QVWDSSGDLWV (SEQ ID NO: 126), AAWDDSLNGLV (SEQ ID NO: 127), QSYDGITVI (SEQ ID NO: 128), QSYDSSNHWV (SEQ ID NO: 129), AVWDDSLSGVV (SEQ ID NO: 131), MIWHSSAYV (SEQ ID NO: 132), NSRDISDNQWQWI (SEQ ID NO: 134), or QSYDSSNHVV (SEQ ID NO: 135). In some embodiments, the antibody of the CAR is fully human or humanized. In other embodiments, the antibody of the CAR is monospecific, bispecific, or multispecific. In further embodiments, the antibody of the CAR is a single chain antibody. In embodiments, X₁, X₂, X₃, or X₄ is a non-polar amino acid residue. In other embodiments, X₁, X₂, X₃, or X₄ is glycine (G), tyrosine (Y), phenylalanine (F), leucine (L), or alanine (A). In some embodiments, X₁, X₂, or X₄ is a hydrophobic amino acid residue, for example X₁, X₂, or X₄ is glycine (G), leucine (L), or alanine (A). In some embodiments, X₃ is a hydrophilic polar amino acid residue. In one embodiment, X₃ is histidine (H). In some embodiments, X₁ is phenylalanine (F), glycine (G) or tyrosine (Y). In further embodiments, X₂ is phenylalanine (F) or leucine (L). In other embodiments, X₃ is histidine (H) or tyrosine (Y). In yet further embodiments, X₄ is serine (S), glycine (G) or alanine (A). In one embodiment, X₈, X₉, X₁₀, or X₁₁ is a non-polar hydrophobic amino acid residue. In some embodiments, X₈, X₉, X₁₀, or X₁₁ is isoleucine (I), proline (P), alanine (A), or phenylalanine (F). In other embodiments, X₈, X₁₀, or X₁₂ is a polar hydrophilic amino acid residue. In yet further embodiments, X₈, X₁₀, or X₁₂ is histidine (H), serine (S), asparagine (N), or threonine (T). In one embodiment, X₈ is alanine (A), isoleucine (I) or serine (S). In one embodiment, X₉ tyrosine (Y), serine (S), proline (P) or alanine (A). In one embodiment, Xio is tyrosine (Y), aspartate (D), isoleucine (I) or histidine (H). In one embodiment, X₁₁ is glycine (G), leucine (L), asparagine (N) or phenylalanine (F). In one embodiment, X₁₂ is isoleucine (I), arginine (R), threonine (T) or histidine (H). In some embodiments, X₅ is a non-polar hydrophobic amino acid residue. In one embodiment, X₅ is glycine (G). In other embodiments, X₅ is a polar hydrophilic amino acid residue. In one embodiment, X₅ is serine (S), asparagine (N), or aspartate (D). In further embodiments, X₆ is a non-polar amino acid residue. In one embodiment, X₆ is tyrosine (Y). In some embodiments, X₆ is a polar hydrophilic amino acid residue. In one embodiment, X₆ is threonine (T), serine (S) or arginine (R). In some embodiments, X₇ X₁₅, X₁₆, X₁₇, X₁₉, X₂₀or X₂₁ is a non-polar hydrophobic amino acid residue. In one embodiment, X₇, X₁₇, or X₂₀is glycine (G). In other embodiments, X₇, X₁₃, X₁₄, X₁₅, X₁₆, X₁₇, X₁₈, X₁₉, or X₂₁ is a polar hydrophilic amino acid residue. In one embodiment, X₇ X₁₄, or X₂₁ is serine (S) or arginine (R). In one embodiment, X₁₃ is serine (S) or threonine (T). In one embodiment, X₁₅ is proline (P). In one embodiment, X₁₅, X₁₇, or X₂₀ is serine (S). In one embodiment, X₁₆ is threonine (T) or arginine (R). In one embodiment, X₁₆ isoleucine (I). In one embodiment, X₁₈ is serine (S) or asparagine (N). In one embodiment, X₁₉ is glycine (G), or alanine (A). In one embodiment, X₁₉ is aspartate (D). In one embodiment, X₂₁ is alanine (A). In one embodiment, X₂₁ is glutamate (E). In some embodiments, transmembrane domain further comprises a stalk region positioned between the extracellular domain and the transmembrane domain. In other embodiments, the transmembrane domain comprises CD28. In some embodiments, the CAR further comprises one or more additional costimulatory molecules positioned between the transmembrane domain and the intracellular signaling domain. In a further embodiment, the costimulatory molecule is CD28, 4-1BB, ICOS, or OX40. In some embodiments, the intracellular signaling domain comprises a CD3 zeta chain. In other embodiments, the antibody of the CAR is a Fab or a scFV.

Aspects of the invention are directed to nucleic acids encoding a CAR described herein. In some embodiments, the nucleic acid encoding the CAR further comprises a nucleic acid encoding a polypeptide positioned after the intracellular signaling domain. In some embodiments, the polypeptide is an antibody a cytokine. In other embodiments, the antibody is a scFV.

Aspects of the invention are directed to a nucleic acid encoding a CAR. In one embodiment, the CAR comprises an intracellular signaling domain, a transmembrane domain and an extracellular domain, further comprising a nucleic acid encoding a polypeptide positioned after the intracellular signaling domain, wherein the polypeptide comprises an isolated monoclonal antibody or antigen-binding fragment thereof that binds to human Programmed death-ligand 1 (PD-L1) protein, wherein the monoclonal antibody or fragment thereof comprises a heavy chain, light chain, or combination thereof. In some embodiments, the heavy chain comprises a CDR1 comprising G-(X1)-T-(X2)-SS-(X3X4) (SEQ ID NO: 47), G-(X₁)-T-(X₂)-(X₁₃X₁₄)-(X₃X₄) (SEQ ID NO: 205), G-(X₁)-TF-(X₁₃X₁₄)-Y-(X₄) (SEQ ID NO: 206), CDR2 comprising I-(X₈X₉X₁₀X₁₁)-G-(X₁₂)-A (SEQ ID NO: 51) or II-(X₁₅)-IFG-(X₁₆)-A (SEQ ID NO: 207), and/or a CDR3 comprising ARGRQMFGAGIDF (SEQ ID NO: 6), ARVHAALYYGMDV (SEQ ID NO: 14), TTGGLGLVYPYYNYIDV (SEQ ID NO: 99), AKVHPVFSYALDV (SEQ ID NO: 100), AEEGAFNSLAI (SEQ ID NO: 101), ARDGSGYDSAGMDD (SEQ ID NO: 102), ARGFGGPDY (SEQ ID NO: 103), ARVHGALYYGMDV (SEQ ID NO: 104), ASGSIVGAAYAFDI (SEQ ID NO: 105), ARDRSEGGFDP (SEQ ID NO: 106), or AEEGAFNSLAI (SEQ ID NO: 107). In another embodiment, the light chain comprises a CDR1 comprising SGSIDSNY (SEQ ID NO: 18), S-(X₁₇X₁₈)I-(X₁₉)-SNY (SEQ ID NO: 208), or NIG-(X₅)-K-(X₂₀) (SEQ ID NO: 48), a CDR2 comprising EDN (SEQ ID NO: 20), (X₂₁)-DN (SEQ ID NO: 209), (X₂₂)-NN (SEQ ID NO: 210), or DD-X₆ (SEQ ID NO: 49), and/or a CDR3 comprising QSYDSNNRHVI (SEQ ID NO: 22), QVWDS-(X₇)-SDHWV (SEQ ID NO: 50), QVWDSSGDLWV (SEQ ID NO: 126), AAWDDSLNGLV (SEQ ID NO: 127), QSYDGITVI (SEQ ID NO: 128), QSYDSSNHWV (SEQ ID NO: 129), AVWDDSLSGVV (SEQ ID NO: 131), MIWHSSAYV (SEQ ID NO: 132), NSRDISDNQWQWI (SEQ ID NO: 134), or QSYDSSNHVV (SEQ ID NO: 135). In some embodiments, the antibody of the CAR is fully human or humanized. In other embodiments, the antibody of the CAR is monospecific, bispecific, or multispecific. In further embodiments, the antibody of the CAR is a single chain antibody. In embodiments, X₁, X₂, X₃, or X₄ is a non-polar amino acid residue. In other embodiments, X₁, X₂, X₃, or X₄ is glycine (G), tyrosine (Y), phenylalanine (F), leucine (L), or alanine (A). In some embodiments, X₁, X₂, or X₄ is a hydrophobic amino acid residue, for example X₁, X₂, or X₄ is glycine (G), leucine (L), or alanine (A). In some embodiments, X₃ is a hydrophilic polar amino acid residue. In one embodiment, X₃ is histidine (H). In some embodiments, X₁ is phenylalanine (F), glycine (G) or tyrosine (Y). In further embodiments, X₂ is phenylalanine (F) or leucine (L). In other embodiments, X₃ is histidine (H) or tyrosine (Y). In yet further embodiments, X₄ is serine (S), glycine (G) or alanine (A). In one embodiment, X₈, X₉, X₁₀, or X₁₁ is a non-polar hydrophobic amino acid residue. In some embodiments, X₈, X₉, X₁₀, or X₁₁ is isoleucine (I), proline (P), alanine (A), or phenylalanine (F). In other embodiments, X₈, X₁₀, or X₁₂ is a polar hydrophilic amino acid residue. In yet further embodiments, X₈, X₁₀, or X₁₂ is histidine (H), serine (S), asparagine (N), or threonine (T). In one embodiment, X₈ is alanine (A), isoleucine (I) or serine (S). In one embodiment, X₉ tyrosine (Y), serine (S), proline (P) or alanine (A). In one embodiment, X₁₀ is tyrosine (Y), aspartate (D), isoleucine (I) or histidine (H). In one embodiment, X₁₁ is glycine (G), leucine (L), asparagine (N) or phenylalanine (F). In one embodiment, X₁₂ is isoleucine (I), arginine (R), threonine (T) or histidine (H). In some embodiments, X₅ is a non-polar hydrophobic amino acid residue. In one embodiment, X₅ is glycine (G). In other embodiments, X₅ is a polar hydrophilic amino acid residue. In one embodiment, X₅ is serine (S), asparagine (N), or aspartate (D). In further embodiments, X₆ is a non-polar amino acid residue. In one embodiment, X₆ is tyrosine (Y). In some embodiments, X₆ is a polar hydrophilic amino acid residue. In one embodiment, X₆ is threonine (T), serine (S) or arginine (R). In some embodiments, X₇ X₁₅, X₁₆, X₁₇, X₁₉, X₂₀, or X₂₁ is a non-polar hydrophobic amino acid residue. In one embodiment, X₇, X₁₇, or X₂₀ is glycine (G). In other embodiments, X₇, X₁₃, X₁₄, X₁₅, X₁₆, X₁₇, X₁₈, X₁₉, or X₂₁ is a polar hydrophilic amino acid residue. In one embodiment, X₇ X₁₄, or X₂₁ is serine (S) or arginine (R). In one embodiment, X₁₃ is serine (S) or threonine (T). In one embodiment, X₁₅ is proline (P). In one embodiment, X₁₅, X₁₇, or X₂₀ is serine (S). In one embodiment, X₁₆ is threonine (T) or arginine (R). In one embodiment, X₁₆ isoleucine (I). In one embodiment, X₁₈ is serine (S) or asparagine (N). In one embodiment, X₁₉ is glycine (G), or alanine (A). In one embodiment, X₁₉ is aspartate (D). In one embodiment, X₂₁ is alanine (A). In one embodiment, X₂₁ is glutamate (E). In some embodiments, transmembrane domain further comprises a stalk region positioned between the extracellular domain and the transmembrane domain. In other embodiments, the transmembrane domain comprises CD28. In some embodiments, the CAR further comprises one or more additional costimulatory molecules positioned between the transmembrane domain and the intracellular signaling domain. In a further embodiment, the costimulatory molecule is CD28, 4-1BB, ICOS, or OX40. In some embodiments, the intracellular signaling domain comprises a CD3 zeta chain. In other embodiments, the antibody of the CAR is a Fab or a scFV.

Aspects of the invention are directed to vectors comprising nucleic acids encoding CARs described herein.

Aspects of the invention are directed to cells hosting vectors comprising nucleic acids encoding CARs described herein.

Aspects of the invention are directed to genetically engineered cells that express the CARS described herein. In one embodiment, the cell expresses and bears on the cell surface membrane a chimeric antigen receptor described herein. In some embodiments, the cell is a T-cell or an NK cell. In further embodiments, the T cell is CD4⁺ or CD8⁺. In other embodiments, the genetically engineered cell comprises a mixed population of CD4⁺ and CD8 cells⁺.

An aspect of the invention is directed to genetically engineered cell which express and bear on the cell surface membrane a chimeric antigen receptor, and which is further engineered to express and secrete a polypeptide, wherein polypeptide is an isolated monoclonal antibody or antigen-binding fragment thereof that binds to human Programmed death-ligand 1 (PD-L1) protein, wherein the monoclonal antibody or fragment thereof comprises a heavy chain, light chain, or combination thereof. In some embodiments, the heavy chain comprises a CDR1 comprising G-(X1)-T-(X2)-SS-(X3X4) (SEQ ID NO: 47), G-(X₁)-T-(X₂)-(X₁₃X₁₄)-(X₃X₄) (SEQ ID NO: 205), G-(X₁)-TF-(X₁₃X₁₄)-Y-(X₄) (SEQ ID NO: 206), CDR2 comprising I-(X₈X₉X₁₀X₁₁)-G-(X₁₂)-A (SEQ ID NO: 51) or II-(X₁₅)-IFG-(X₁₆)-A (SEQ ID NO: 207), and/or a CDR3 comprising ARGRQMFGAGIDF (SEQ ID NO: 6), ARVHAALYYGMDV (SEQ ID NO: 14), TTGGLGLVYPYYNYIDV (SEQ ID NO: 99), AKVHPVFSYALDV (SEQ ID NO: 100), AEEGAFNSLAI (SEQ ID NO: 101), ARDGSGYDSAGMDD (SEQ ID NO: 102), ARGFGGPDY (SEQ ID NO: 103), ARVHGALYYGMDV (SEQ ID NO: 104), ASGSIVGAAYAFDI (SEQ ID NO: 105), ARDRSEGGFDP (SEQ ID NO: 106), or AEEGAFNSLAI (SEQ ID NO: 107). In another embodiment, the light chain comprises a CDR1 comprising SGSIDSNY (SEQ ID NO: 18), S-(X₁₇X₁₈)I-(X₁₉)-SNY (SEQ ID NO: 208), or NIG-(X₅)-K-(X₂₀) (SEQ ID NO: 48), a CDR2 comprising EDN (SEQ ID NO: 20), (X₂₁)-DN (SEQ ID NO: 209), (X₂₂)-NN (SEQ ID NO: 210), or DD-X₆ (SEQ ID NO: 49), and/or a CDR3 comprising QSYDSNNRHVI (SEQ ID NO: 22), QVWDS-(X₇)-SDHWV (SEQ ID NO: 50), QVWDSSGDLWV (SEQ ID NO: 126), AAWDDSLNGLV (SEQ ID NO: 127), QSYDGITVI (SEQ ID NO: 128), QSYDSSNHWV (SEQ ID NO: 129), AVWDDSLSGVV (SEQ ID NO: 131), MIWHSSAYV (SEQ ID NO: 132), NSRDISDNQWQWI (SEQ ID NO: 134), or QSYDSSNHVV (SEQ ID NO: 135). In some embodiments, the antibody of the CAR is fully human or humanized. In other embodiments, the antibody of the CAR is monospecific, bispecific, or multispecific. In further embodiments, the antibody of the CAR is a single chain antibody. In embodiments, X₁, X₂, X₃, or X₄ is a non-polar amino acid residue. In other embodiments, X₁, X₂, X₃, or X₄ is glycine (G), tyrosine (Y), phenylalanine (F), leucine (L), or alanine (A). In some embodiments, X₁, X₂, or X₄ is a hydrophobic amino acid residue, for example X₁, X₂, or X₄ is glycine (G), leucine (L), or alanine (A). In some embodiments, X₃ is a hydrophilic polar amino acid residue. In one embodiment, X₃ is histidine (H). In some embodiments, X₁ is phenylalanine (F), glycine (G) or tyrosine (Y). In further embodiments, X₂ is phenylalanine (F) or leucine (L). In other embodiments, X₃ is histidine (H) or tyrosine (Y). In yet further embodiments, X₄ is serine (S), glycine (G) or alanine (A). In one embodiment, X₈, X₉, X₁₀, or X₁₁ is a non-polar hydrophobic amino acid residue. In some embodiments, X₈, X₉, X₁₀, or X₁₁ is isoleucine (I), proline (P), alanine (A), or phenylalanine (F). In other embodiments, X₈, X₁₀, or X₁₂ is a polar hydrophilic amino acid residue. In yet further embodiments, X_{8,} X₁₀, or X₁₂ is histidine (H), serine (S), asparagine (N), or threonine (T). In one embodiment, X₈ is alanine (A), isoleucine (I) or serine (S). In one embodiment, X₉ tyrosine (Y), serine (S), proline (P) or alanine (A). In one embodiment, Xio is tyrosine (Y), aspartate (D), isoleucine (I) or histidine (H). In one embodiment, X₁₁ is glycine (G), leucine (L), asparagine (N) or phenylalanine (F). In one embodiment, X₁₂ is isoleucine (I), arginine (R), threonine (T) or histidine (H). In some embodiments, X₅ is a non-polar hydrophobic amino acid residue. In one embodiment, X₅ is glycine (G). In other embodiments, X₅ is a polar hydrophilic amino acid residue. In one embodiment, X₅ is serine (S), asparagine (N), or aspartate (D). In further embodiments, X₆ is a non-polar amino acid residue. In one embodiment, X₆ is tyrosine (Y). In some embodiments, X₆ is a polar hydrophilic amino acid residue. In one embodiment, X₆ is threonine (T), serine (S) or arginine (R). In some embodiments, X₇ X₁₅, X₁₆, X₁₇, X₁₉, X₂₀, or X₂₁ is a non-polar hydrophobic amino acid residue. In one embodiment, X₇, X₁₇, or X₂₀ is glycine (G). In other embodiments, X₇, X₁₃, X₁₄, X₁₅, X₁₆, X₁₇, X₁₈, X₁₉, or X₂₁ is a polar hydrophilic amino acid residue. In one embodiment, X₇ X₁₄, or X₂₁ is serine (S) or arginine (R). In one embodiment, X₁₃ is serine (S) or threonine (T). In one embodiment, X₁₅ is proline (P). In one embodiment, X₁₅, X₁₇, or X₂₀ is serine (S). In one embodiment, X₁₆ is threonine (T) or arginine (R). In one embodiment, X₁₆ isoleucine (I). In one embodiment, X₁₈ is serine (S) or asparagine (N). In one embodiment, X₁₉ is glycine (G), or alanine (A). In one embodiment, X₁₉ is aspartate (D). In one embodiment, X₂₁ is alanine (A). In one embodiment, X₂₁ is glutamate (E).

An aspect of the invention is directed to a method of treating cancer in a subject. In some embodiments, the method comprises administering to a subject in need thereof a therapeutically effective amount of a composition comprising an antibody described herein, a bispecific antibody described herein, the pharmaceutical compositions described herein, or the CAR compositions described herein. In one embodiment, the method further comprises administering to the subject a chemotherapeutic agent.

Other objects and advantages of this invention will become readily apparent from the ensuing description.

### BRIEF DESCRIPTION OF THE FIGURES

FIG. 1 shows a schematic of a bispecific GITR-PDL1 light chain fusion.

FIG. 2 are graphs showing FACS plots and binding curves for PD-L1 antibodies.

FIG. 3 shows FACS binding curves with 293T cells stably expressing PD-L1. Antibodies were detected via anti-hFc secondary.

FIG. 4 is a schematic that shows the kinetic measurements for aPDL1 antibodies (top image). Based on a series of previous competition matrices, representative clones were used in a final matrix (bottom image).

FIG. 5 is a graph showing negative background binding of the anti-PDL1 scFv-Fcs to 293T cells.

FIG. 6 is a graph showing results from a Mixed lymphocyte reaction (MLR) assay to test biological activity of anti-PDL1 clones. IFNy was detected by ELISA as a measure of T cell activation. The development of ELISA plates indicate some clones are comparable to atez.

FIG. 7 is a bar graph showing MLR results with αPD-L1 antibody.

FIG. 8 is a bar graph showing MLR results with αPD-L1 antibody (150 nM).

FIG. 9 is a bar graph showing MLR results with αPD-L1 antibody (150 nM).

FIG. 10 is a schematic of the variable region heavy chain germline alignments (amino acid sequences) (SEQ ID NOS 252, 54, 56, 16, 62, 66, 68, 74, 66, 52, 253-254, 70, 255, 64, 64, 256, 78, 257, 60, 258, 72, 259, and 80, respectively, in order of appearance).

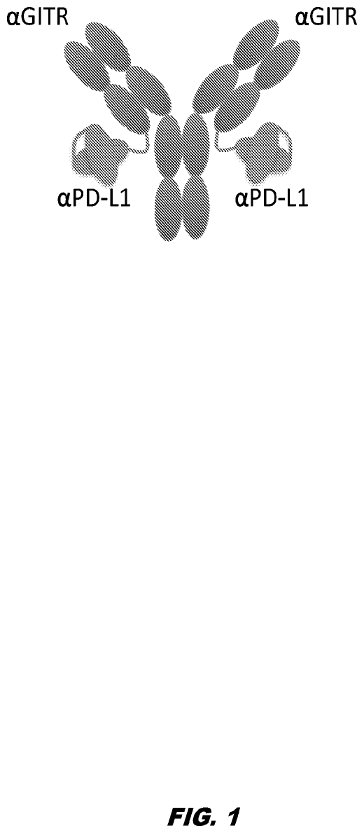

### DETAILED DESCRIPTION OF THE INVENTION

### Abbreviations and Definitions

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### PD-L1

Programmed T cell death 1 (PD-1) is a trans-membrane protein found on the surface of T cells, which, when bound to programmed T cell death ligand 1 (PD-L1) on tumor cells, results in suppression of T cell activity and reduction of T cell-mediated cytotoxicity. Thus, PD-1 and PD-L1 are immune down-regulators or immune checkpoint “off switches.”

The immune system must achieve a balance between effective responses to eliminate pathogenic entities and maintaining tolerance to prevent autoimmune disease. T cells are central to preserving this balance, and their proper regulation is primarily coordinated by the B7-CD28 family of molecules. Interactions between B7 family members, which function as ligands, and CD28 family members, which function as receptors, provide critical positive signals that not only initiate, augment and sustain T cell responses, but also contribute key negative signals that limit, terminate and/or attenuate T cell responses when appropriate. PD-1 is a member of the CD28 family.

Binding between PD-L1 and PD-1 has a profound effect on the regulation of T cell responses. Specifically, PD-L1/PD-1 interaction inhibits T cell proliferation and production of effector cytokines that mediate T cell activity and immune response, such as IL-2 and IFN-γ. This negative regulatory function is important for preventing T cell-mediated autoimmunity and immunopathology. However, the PD-1/PD-L1 axis has also been shown to play a role in T cell exhaustion, whereby the negative regulatory function inhibits T cell response to the detriment of the host. Prolonged or chronic antigenic stimulation of T cells can induce negative immunological feedback mechanisms which inhibit antigen-specific responses and results in immune evasion of pathogens. T cell exhaustion can also result in progressive physical deletion of the antigen-specific T cells themselves. T cell expression of PD-1 is up-regulated during chronic antigen stimulation, and its binding to PD-L1 results in a blockade of effector function in both CD4+ (T helper cells) and CD8+ (cytotoxic T lymphocytes or CTL) T cells, thus implicating the PD-1/PD-L1 interaction in the induction of T cell exhaustion.

More recently, studies showed that some chronic viral infections and cancers have developed immune evasion tactics that specifically exploit the PD-1/PD-L1 axis by causing PD-1/PD-L1-mediated T cell exhaustion. Many human tumor cells and tumor-associated antigen presenting cells express high levels of PD-L1, which suggests that the tumors induce T cell exhaustion to evade anti-tumor immune responses. During chronic HIV infection, for example, HIV-specific CD8+ T cells are functionally impaired, showing a reduced capacity to produce cytokines and effector molecules as well as a diminished ability to proliferate. Studies have shown that PD-1 is highly expressed on HIV-specific CD8+ T cells of HIV infected individuals, indicating that blocking the PD-1/PD-L1 pathway may have therapeutic potential for treatment of HIV infection and AIDS patients. Taken together, agents that block the PD-1/PD-L1 pathway will provide a new therapeutic approach for a variety of cancers, HIV infection, and/or other diseases and conditions that are associated with T-cell exhaustion. Therefore, there exists an urgent need for agents that can block or prevent PD-1/PD-L1 interaction.

PD-L1 overexpression has been detected in different cancers. For example, in breast cancer, PD-L1 is overexpressed and associated with high-risk prognostic factors. In renal cell carcinoma, PD-L1 is upregulated and increased expression of PD-1 has also been found in tumor infiltrating leukocytes. Anti-PD-L1 and anti-PD-1 antibodies have demonstrated some clinical efficacy in phase I trials for renal cell carcinoma. Therapeutic agents that can bind to PD-1 or PD-L1 may be useful for specifically targeting tumor cells. Agents that are capable of blocking the PD-1/PD-L1 interaction may be even more useful in treating cancers that have induced T cell exhaustion to evade anti-tumor T cell activity. Use of such agents, alone or in combination with other anti-cancer therapeutics, can effectively target tumor cells that overexpress PD-L1 and increase anti-tumor T cell activity, thereby augmenting the immune response to target tumor cells.

PD-1 and PD-L1 can also be upregulated by T cells after chronic antigen stimulation, for example, by chronic infections. During chronic HIV infection, HIV-specific CD8+ T cells are functionally impaired, showing a reduced capacity to produce cytokines and effector molecules as well as a diminished ability to proliferate. PD-1 is highly expressed on HIV-specific CD8+ T cells of HIV infected individuals. Therefore, blocking this pathway may enhance the ability of HIV-specific T cells to proliferate and produce cytokines in response to stimulation with HIV peptides, thereby augmenting the immune response against HIV. Other chronic infections may also benefit from the use of PD-1/PD-L1 blocking agents, such as chronic viral, bacterial or parasitic infections.

Aspects of the invention provide isolated monoclonal antibodies specific against PDL-1. The term “isolated” as used herein with respect to cells, nucleic acids, such as DNA or RNA, refers to molecules separated from other DNAs or RNAs, respectively, that are present in the natural source of the macromolecule. The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and would not be found in the natural state. “Isolated” can also refer to cells or polypeptides which are isolated from other cellular proteins or tissues. Isolated polypeptides can include both purified and recombinant polypeptides. The isolated antibodies were identified through the use of a 27 billion human single-chain antibody (scFv) phage display library via paramagnetic proteoliposomes, by using PDL-1 as a library selection target. These antibodies represent a new class of monoclonal antibodies against PD-L1.

Five unique recombinant monoclonal PD-L1 antibodies are described herein. These include 40 mut, 50-6B6.1 mut, 50-6B6.2, 50-7B3, and 50-5B9. “Recombinant” as it pertains to polypeptides (such as antibodies) or polynucleotides can refer to a form of the polypeptide or polynucleotide that does not exist naturally, a non-limiting example of which can be created by combining polynucleotides or polypeptides that would not normally occur together.

The nucleic acid and amino acid sequence of the monoclonal PD-L1 antibodies are provided below; the amino acid sequences of the heavy and light chain complementary determining regions (CDRs) of the PD-L1 antibodies are underlined (CDR1), underlined and bolded (CDR2), or underlined, italicized, and bolded (CDR3) below:

TABLE 1
Ab 40 mut Variable Region amino acid sequences

V_{H} chain of Ab 40 mut VH (HV1-69*06)
QVQLVQSGAEVKKPGSSVKVSCKASGGTFSSYAISWVRQAPGQGLEWM
GG [symbol] NYAQKFQGRVTITADKSTSTAYMELSSLRSEDTAVYYC [symbol] [symbol]
WGPGTLVTVSS
(SEQ ID NO: 8)
V_{L} chain of Ab 40 mut VL (LV6-57*01)
NFMLTQPHSVSESPGKTVTISCTRSSGSIDSNYVQWYQQRPGSAPTTV
IY [symbol] QRPSGVPDRFSGSIDSSSNSASLTISGLKTEDEADYYC [symbol] [symbol]
FGGGTKLTVL
(SEQ ID NO: 24)

TABLE 2B
Ab 50-6B6.1 mut Variable Region
amino acid sequences

V_{H} chain of Ab 50-6B6.1 mut VH (HV1-18*01)
QVQLVQSGGEVKKPGASVKVSCKASGYTLSSHGITWVRQAPGQGLEWM
GW [symbol] [symbol] NAQKVEDRVTMTTDTSTNTAYMELRSLTADDTAVYYC [symbol] [symbol] [symbol]
WGQGTLVTVSS
(SEQ ID NO: 16)
V_{L} chain of Ab 50-6B6.1 mutVL (LV3-21*02)
SYELTQPPSVSLAPGQSARISCGGDNIGSKGVHWYQQKPGQAPVVVVY [symbol]
DRPSGIPERFSGSNSGNTATLTISRVEAGDEADYYC [symbol] [symbol]
FGGGTKLTVL
(SEQ ID NO: 31)

TABLE 3B
Ab 50-6B6.2 Variable Region amino acid sequences

V_{H} chain of Ab 50-6B6.2 VH (HV1-18*01)
QVQLVQSGGEVKKPGASVKVSCKASGYTLSSHGITWVRQAPGQGLEWM
GW [symbol] [symbol] SNAQKVEDRVTMTTDTSTNTAYMELRSLTADDTAVYYC [symbol] [symbol] [symbol]
WGQGTLVTVSS
(SEQ ID NO: 16)
V_{L} chain of Ab 50-6B6.2VL (LV3-21*02)
LPVLTQPPSVSAAPGQTARISCGGSNIGDKGVHWYQQKPGQAPVLVIY [symbol]
DRPSGIPERFSGSNSGNTATLTISRVEAGDEADYYC [symbol] [symbol]
FGGGTKLTVL
(SEQ ID NO: 38)

TABLE 4B
Ab50-6B6.2 Variable Region amino acid sequences

V_{H} chain of Ab 50-7B3VH (HV1-18*01)
QVQLVQSGGEVKKPGASVKVSCKASGYTLSSHGITWVRQAPGQGLEWM
GW [symbol] [symbol] SNAQKVEDRVTMTTDTSTNTAYMELRSLTADDTAVYYC [symbol] [symbol]
DVWGQGTLVTVSS
(SEQ ID NO: 16)
V_{L} chain of Ab 50-7B3 VL (LV3-21*02)
SYELTQPPSVSVAPGQTARITCGGNNIGNKGVHWYQQKPGQAPVLVVY [symbol]
DRPSGIPERFSGSNSGNTATLTISRVEAGDEADYYC [symbol] [symbol]
FGGGTKLTVL
(SEQ ID NO: 42)

TABLE 5B
Ab 50-5B9 Variable Region amino acid sequences

V_{H} chain of Ab 50-5B9VH (HV1-18*01)
QVQLVQSGGEVKKPGASVKVSCKASGYTLSSHGITWVRQAPGQGLEWM
GW [symbol] [symbol] SNAQKVEDRVTMTTDTSTNTAYMELRSLTADDTAVYYC [symbol] [symbol] [symbol]
WGQGTLVTVSS
(SEQ ID NO: 16)
V_{L} chain of Ab 50-5B9 VL (LV3-21*02)
LPVLTQPPSVSVALGQTARITCRGNNIGGKGVHWYQQKPGQAPVLVVY [symbol]
SRRSGIPERFSGSHSGSAATLTISRVEAGDEADYYC [symbol] [symbol]
FGGGTKLTVL
(SEQ ID NO: 46)

TABLE 6
Ab 14C61 Variable Region amino acid sequences

V_{H} chain of14C61 (HV1-18*, HJ6*02, HD2-2*01)
EVQLVQSGGEVKKPGASVKVSCKASGYTLSSHGITWVRQAPGQGLEWMG
W [symbol] [symbol] SNAQKVEDRVTMTTDTSTNTAYMELRSLTPDDTAVYYC [symbol] [symbol] [symbol]
WGQGTLVTVSS
(SEQ ID NO: 52)
V_{L} chain of 14C61 (LV3-21*02, LJ3*02)
SYELTQPPSVSVAPRQTAKITCTRDNIESRSVNWYQQRAGQAPAVIVY [symbol]
ERPSGITVRYSGSNSGNTATLTISRVEAGDEADYYC [symbol] [symbol]
FGGGTKLTVL
(SEQ ID NO: 53)

TABLE 7
Ab 1A2 Variable Region amino acid sequences

V_{H} chain of 1A2 (HV1-18*01, HJ6*02, HD2-2*01)
QVQLVQSGGEVKKPGASVKVSCKASGYTLSSHGITWVRQAPGQGLEWM
GW [symbol] [symbol] SNAQKVEDRVTMTTDTSTNTAYMELRSLTADDTAVYYC [symbol] [symbol] [symbol]
WGQGTTVTVSS
(SEQ ID NO: 54)
V_{L} chain of 1A2 (LV3-21*02, LJ3*02)
QSVLTQPPSVSVAPGQTARITCGGNNIGSKGVHWYQQKPGQAPVLVVY [symbol]
DRPSGIPERFSGSNSGNTATLTISRVEAGDEADYYC [symbol] [symbol]
FGGGTKLTVL
(SEQ ID NO: 55)

TABLE 8
Ab 1A3 Variable Region amino acid sequences

V_{H} chain of 1A3 (HV1-18*01, HJ6*02, HD2-2*01)
EVQLVQSGAEVKKPGASVKVSCKASGYTLSSHGITWVRQAPGQGLEWM
GW [symbol] [symbol] SNAQKVEDRVTMTTDTSTNTAYMELRSLTPDDTAVYYC [symbol] [symbol] [symbol]
WGQGTLVTVSS
(SEQ ID NO: 56)
V_{L} chain of 1A3 (LV3-21*02, LJ3*02)
LPVLTQPPSVSVAPGQTARITCGGNNIGSKSVHWYQQKPGQAPVLVVY [symbol]
DRPSGIPERFSGSNSGNTATLTISRVEAGDEADYYC [symbol] [symbol]
FGGGTKLTVL
(SEQ ID NO: 57)

TABLE 9
Ab 1A6 Variable Region amino acid sequences

V_{H} chain of 1A6 (HV1-18*01, HJ6*02, HD2-2*01)
QVQLVQSGGEVKKPGASVKVSCKASGYTLSSHGITWVRQAPGQGLEWM
GW [symbol] [symbol] SNAQKVEDRVTMTTDTSTNTAYMELRSLTADDTAVYYC [symbol] [symbol] [symbol]
WGQGTLVTVSS
(SEQ ID NO: 16)
V_{L} chain of 1A6 (LV3-21*02, LJ3*02)
QSVLTQPPSVSVAPGQTARITCGGNNIGSKGVHWYQQKPGQAPVLVVY [symbol]
DRPSGIPERFSGSNSGNTATLTISRVEAGDEADYYC [symbol] [symbol]
FGGGTKLTVL
(SEQ ID NO: 59)

TABLE 10
Ab 1B4 Variable Region amino acid sequences

V_{H} chain of 1B4 (HV3-15*07, HJ6*03, HD3-16*01)
QVQLVQSGGGLVKPGGSLRLSCVGSDFAFSSAWMNWVRQAPGKGLEWV
GR [symbol] [symbol] DYAAPVKDRFIISRDDSKNTLYLEMNSLKTEDTGVY
YC [symbol] [symbol] [symbol] WGEGTTVTVSS
(SEQ ID NO: 60)
V_{L} chain of 1B4 (LV1-47*01, LJ1*01)
LPVLTQPPSASGTPGQRVTISCSGSSSNIGSNYVYWYQQLPGTAPKLL
IY [symbol] QRPSGVPDRFSGSKSGTSASLAISGLRSEDEADYYC

[symbol]

[symbol]

(SEQ ID NO: 61)

TABLE 11
Ab 1C1 Variable Region amino acid sequences

V_{H} chain of 1C1 (HV1-18*01, HJ3*01, HD2-21*02)
QVQLVQSGAEVKKPGASVKVSCKASGYTFTSYGISWVRQAPGQGLE
WMGW [symbol] [symbol] AFAQILEGRVTMTTDTSTNTAYMELRNLTFDDTA
VYFC [symbol] [symbol] WGQGTLVTVSS
(SEQ ID NO: 62)
V_{L} chain of 1C1 (LV6-57*01, LJ2*01)
NFMLTQPHSVSESPGKTVTISCTRSSGSIASNYVQWYQQRPGSSPT
TVIY [symbol] QRPSGVPDRFSGSIDTSSNSASLTISGLKTKDEADYYC [symbol]
FGGGTKLTVL
(SEQ ID NO: 63)

TABLE 12
Ab 1C4 Variable Region amino acid sequences

V_{H} chain of 1C4 (HV1-69*06, HJ3*02, HD3-10*01)
QVQLVQSGAEVKKPGSSVKVSCKASGGTFSRYALTWVRQAPGQGLE
WMGG [symbol] [symbol] NYAQKFQGRVTITADKSTSTAYMELGSLTSDDTA
VYYC [symbol] [symbol] WGQGTMVTVSS
(SEQ ID NO: 64)
V_{L} chain of 1C4 (LV6-57*03, LJ3*02)
NFMLTQPHSVSESPGKTVTISCTRSSGSIASNYVQWYQQRPGSAPT
TVIF [symbol] QRPSGVPARFSGSIDSSSNSASLTISGLKTEDEADYYC [symbol] [symbol]
FGGGTQLTVL
(SEQ ID NO: 65)

TABLE 13
Ab 1C6 Variable Region amino acid sequences

V_{H} chain of 1C6 (HV1-18*01, HJ6*02, HD2-2*01)
QVQLVQSGGEVKKPGASVKVSCKASGYTLSSHGITWVROAPGQGLE
WMGW [symbol] [symbol] SNAQKVEDRVTMTTDTSTNTAYMELRSLTPDDTA
VYYC [symbol] [symbol] [symbol] WGQGTLVTVSS
(SEQ ID NO: 66)
V_{L} chain of 1C6 (LV3-21*02, LJ3*02)
LPVLTQPPSVSVAPGQTARITCGGNNIGSKSVHWYQQKPGQAPVLV
VY [symbol] DRPSGIPERFSGSNSGNTATLTINRVEAGDEADYYC [symbol] [symbol]
FGGGTKLTVL
(SEQ ID NO: 67)

TABLE 14
Ab 1D1 Variable Region amino acid sequences

V_{H} chain of 1D1 (HV1-18*01, HJ6*02, HD2-2*01)
QVQLVQSGAEVKKPGASVKVSCKASGYTFTSYGISWVRQAPGQGLE
WMGW [symbol] [symbol] SNAQKVEDRVTMTTDTSTNTAYMELRNLTTDDTA
VYYC [symbol] [symbol] [symbol] WGQGTTVTVSS
(SEQ ID NO: 68)
V_{L} chain of 1D1 (LV3-21*02, LJ3*02)
SYELTQPPSVSVAPGQTARITCGGDNIGSKGVHWYQQTPGQAPVLV
VY [symbol] DRPSGIPERFSGSKSGNTATLTISRVEAGDEADYYC [symbol] [symbol]
FGGGTRVTVL
(SEQ ID NO: 69)

TABLE 15
Ab 1D2 Variable Region amino acid sequences

V_{H} chain of 1D2 (HV1-69*01, HJ6*02, HD5-12*01)
QVQLVQSGAEVKKPGSSVKVSCKASGGTFSSYAISWVRQAPGQGLE
WMGG [symbol] [symbol] NYAQKFQGRVTITADESTSTAYMELSSLRSEDTA
VYYC [symbol] [symbol] WGQGTLVTVSS
(SEQ ID NO: 70)
V_{L} chain of 1D2 (LV1-47*02, LJ2*01)
QPGLTQPPSASGTPGQTVTISCSGSRSNIGSNYVYWYQQFPGTAPK
LLIF [symbol] QRPSGVPDRFSGSKSGTSASLAISGLRSEDEADYYC [symbol] [symbol]
FGGGTKLTVL
(SEQ ID NO: 71)

TABLE 16
Ab 1D4 Variable Region amino acid sequences

V_{H} chain of 1D4 (HV3-30*04, HJ4*02, HD2-21*01)
QVQLVQSGGGVVQPGRSLRLSCAASGFTFSSYAIHWVRQAPGKGLE
WITT [symbol] [symbol] YYADSVKGRFTISRDNPKNTLYLQMNSLRAEDTA
VYYC [symbol] WGQGTLVTVSS
(SEQ ID NO: 72)
V_{L} chain of 1D4 (LV5-45*03, LJ*01)
LPVLTQPSSLSASPGASASLTCTLRSGINVGTYRIYWYQQKPGSPP
QYLLR [symbol] [symbol] QQGSGVPSRFSGSKDASANAGILLISGLQSEDEA
DYYC [symbol] FGTGTKVTVL
(SEQ ID NO: 73)

TABLE 17
Ab 1E1 Variable Region amino acid sequences

V_{H} chain of 1E1 (HV1-18*01, HJ6*02, HD2-2*01)
QVQLVQSGVEMKKPGASVRVSCKGSGYTFSSYGISWVRQAPGQGLE
WMGW [symbol] [symbol] SNAQKLEDRVTMTTDTSTNTAYMELRSLTSDDTA
VYYC [symbol] [symbol] [symbol] WGQGTTVTVSS
(SEQ ID NO: 74)
V_{L} chain of 1E1 (LV3-21*02, LJ3*02)
SYELTQPPSVSVAPGQTARIPCGANNIGGKSVHWYQQKPGQAPVLV
VY [symbol] DRPSGIPERFSGSNSGNTATLTISRVEAGDEADYYC [symbol] [symbol]
FGGGTKLTVL
(SEQ ID NO: 75)

TABLE 18
Ab 1F1 Variable Region amino acid sequences

V_{H} chain of 1F1 (HV1-18*01, HJ6*02, HD2-2*01)
QVQLVQSGGEVKKPGASVKVSCKASGYTLSSHGITWVRQAPGQGLE
WMGW [symbol] [symbol] SNAQKVEDRVTMTTDTSTNTAYMELRSLTPDDTA
VYYC [symbol] [symbol] [symbol] WGQGTLVTVSS
(SEQ ID NO: 76)
V_{L} chain of 1F1 (LV3-21*02, LJ3*02)
QPVLTQPPSVSVAPGQTARITCGGDNIGSKGVHWLQQKPGQAPVLV
VY [symbol] DRPSGIPERFSGSNSGSTATLTISRVEAGDEADYYC [symbol] [symbol]
FGGGTKLTVL
(SEQ ID NO: 77)

TABLE 19
Ab 1G1 Variable Region amino acid sequences

V_{H} chain of 1G1 (HV1-69*09, HJ3*02, HD1-26*01)
EVQLVQSGAEVKKPGSSVRVSCKASGGTFSSYAISWVRQAPGQGLE
WMGR [symbol] [symbol] NYAQKFQGRVTITADKSTSTAYMELSSLRSEDTA
VYYC [symbol] [symbol] WGQGTTVTVSS
(SEQ ID NO: 78)
V_{L} chain of 1G1 (LV3-21*03, LJ3*02)
QSVLTQPPSVSVAPGKAANLNCGGKNIGGRVVHWYQQRPGQAPVLV
IY [symbol] DRPSGIPERFSGSNSGNTATLTITDVEVGDEADYYC [symbol] [symbol]
FGGGTTLTVL
(SEQ ID NO: 79)

TABLE 20
Ab 1H2 Variable Region amino acid sequences

V_{H} chain of 1H2 (HV3-74*02, HJ5*02, HD1-1*01)
QVQLVQSGGGLVKPGGSLRLSCAASGFTFSSYSMNWVRQAPGKGLV
WISR [symbol] [symbol] TYADSVKGRFTISRDNAKNTLYLQMNSLRAEDTG
VYYC [symbol] [symbol] WGQGTLVTVSS
(SEQ ID NO: 80)
V_{L} chain of 1H2 (LV3-19*01, LJ3*02)
SSELTQDPAVSVALGQTVRITCQGDSLRSYYASWYQQKPGQAPVLV
IYGKNNRPSGIPDRFSGSTSGNTASLTITGAQAEDEADYYC [symbol] [symbol]
FGGGTKLAVL
(SEQ ID NO: 81)

TABLE 21
Ab 1H5 Variable Region amino acid sequences

V_{H} chain of 1H5 (HV1-69*06, HJ3*02, HD3-10*01)
QVQLVQSGAEVKKPGSSVKVSCKASGGTFSRYALTWVRQAPGQGLE
WMGG [symbol] [symbol] NYAQKFQGRVTITADKSTSTAYMELGSLTSDDTA
VYYC [symbol] [symbol] WGQGTMVTVSS
(SEQ ID NO: 82)
V_{L} chain of 1H5 (LV6-57*01, LJ2*01)
NFMLTQPHSVSGSPGETVTISCTRSSGSIASHFVQWYQQRPGSSPT
TVIF [symbol] QRPSGVPDRISGSIDTSSNSASLSISGLKTEDEADYYC [symbol]
FGGGTKLTVL
(SEQ ID NO: 83)

The amino acid sequences of the heavy and light chain complementary determining regions of the PDL-1 antibodies are shown in Table 6A-B below:

TABLE 6A
Heavy chain (V_{H}) complementary determining
regions (CDRs) of the PDL-1 antibodies

| Sequence | | | |
|---|---|---|---|
| ID | V_{H} CDR1 | V_{H} CDR2 | V_{H} CDR3 |
| 40 mut | GGTFSSYA | IIPIFGTA | ARGRQMFGAGIDF |
| | (SEQ ID NO: 2) | (SEQ ID NO: 4) | (SEQ ID NO: 6) |
| 50-6B6.1 | GYTLSSHG | ISAHNGHA | ARVHAALYYGMDV |
| mut | (SEQ ID NO: 10) | (SEQ ID NO: 12) | (SEQ ID NO: 14) |
| 50-6B6.2 | GYTLSSHG | ISAHNGHA | ARVHAALYYGMDV |
| | (SEQ ID NO: 10) | (SEQ ID NO: 12) | (SEQ ID NO: 14) |
| 50-7B3 | GYTLSSHG | ISAHNGHA | ARVHAALYYGMDV |
| | (SEQ ID NO: 10) | (SEQ ID NO: 12) | (SEQ ID NO: 14) |
| 50-5B9 | GYTLSSHG | ISAHNGHA | ARVHAALYYGMDV |
| | (SEQ ID NO: 10) | (SEQ ID NO: 12) | (SEQ ID NO: 14) |
| 14C61 | GYTLSSHG | ISAHNGHA | ARVHAALYYGMDV |
| | (SEQ ID NO: 10) | (SEQ ID NO: 12) | (SEQ ID NO: 14) |
| 1A2 | GYTLSSHG | ISAHNGHA | ARVHAALYYGMDV |
| | (SEQ ID NO: 10) | (SEQ ID NO: 12) | (SEQ ID NO: 14) |
| 1A3 | GYTLSSHG | ISAHNGHA | ARVHAALYYGMDV |
| | (SEQ ID NO: 10) | (SEQ ID NO: 12) | (SEQ ID NO: 14) |
| 1A6 | GYTLSSHG | ISAHNGHA | ARVHAALYYGMDV |
| | (SEQ ID NO: 10) | (SEQ ID NO: 12) | (SEQ ID NO: 14) |
| 1B4 | DFAFSSAW | IKSKTDGETT | TTGGLGLVYPYYNYIDV |
| | (SEQ ID NO: 84) | (SEQ ID NO: 91) | (SEQ ID NO: 99) |
| 1C1 | GYTFTSYG | TSPHNGLT | AKVHPVFSYALDV |
| | (SEQ ID NO: 85) | (SEQ ID NO: 92) | (SEQ ID NO: 100) |
| 1C4 | GGTFSRYA | IIPIFGRA | AEEGAFNSLAI |
| | (SEQ ID NO: 86) | (SEQ ID NO: 93) | (SEQ ID NO: 101) |
| 1C6 | GYTLSSHG | ISAHNGHA | ARVHAALYYGMDV |
| | (SEQ ID NO: 10) | (SEQ ID NO: 12) | (SEQ ID NO: 14) |
| 1D1 | GYTFTSYG | ISAYNGHA | ARVHAALYYGMDV |
| | (SEQ ID NO: 85) | (SEQ ID NO: 94) | (SEQ ID NO: 14) |
| 1D2 | GGTFSSYA | IIPIFGTA | ARDGSGYDSAGMDD |
| | (SEQ ID NO: 87) | (SEQ ID NO: 95) | (SEQ ID NO: 102) |
| 1D4 | GFTFSSYA | ISYDGSNK | ARGFGGPDY |
| | (SEQ ID NO: 88) | (SEQ ID NO: 96) | (SEQ ID NO: 103) |
| 1E1 | GYTFSSYG | ISAHNGHA | ARVHGALYYGMDV |
| | (SEQ ID NO: 89) | (SEQ ID NO: 12) | (SEQ ID NO: 104) |
| 1F1 | GYTLSSHG | ISAHNGHA | ARVHAALYYGMDV |
| | (SEQ ID NO: 10) | (SEQ ID NO: 12) | (SEQ ID NO: 14) |
| 1G1 | GGTFSSYA | IIPILGIA | ASGSIVGAAYAFDI |
| | (SEQ ID NO: 87) | (SEQ ID NO: 97) | (SEQ ID NO: 105) |
| 1H2 | GFTFSSYS | IISDGSAT | ARDRSEGGFDP |
| | (SEQ ID NO: 90) | (SEQ ID NO: 98) | (SEQ ID NO: 106) |
| 1H5 | GGTFSRYA | IIPIFGRA | AEEGAFNSLAI |
| | (SEQ ID NO: 86) | (SEQ ID NO: 93) | (SEQ ID NO: 107) |

TABLE 6B
Light chain (V_{L}) complementary determining
regions (CDRs) of the PDL-1 antibodies

| Sequence | | | |
|---|---|---|---|
| ID | V_{L} CDR1 | V_{L} CDR2 | V_{L} CDR3 |
| 40 mut | SGSIDSNY | EDN | QSYDSNNRHVI |
| | (SEQ ID NO: 18) | (SEQ ID NO: 20) | (SEQ ID NO: 22) |
| 50-6B6.1 | NIGSKG | DDR | QVWDSGSDHWV |
| mut | (SEQ ID NO: 26) | (SEQ ID NO: 28) | (SEQ ID NO: 30) |
| 50-6B6.2 | NIGDKG | DDS | QVWDSSSDHWV |
| | (SEQ ID NO: 33) | (SEQ ID NO: 35) | (SEQ ID NO: 37) |
| 50-7B3 | NIGNKG | DDS | QVWDSSSDHWV |
| | (SEQ ID NO: 40) | (SEQ ID NO: 35) | (SEQ ID NO: 37) |
| 50-5B9 | NIGGKG | DDY | QVWDSSSDHWV |
| | (SEQ ID NO: 44) | (SEQ ID NO: 45) | (SEQ ID NO: 37) |
| 14C61 | NIESRS | DDT | QVWDSSGDLWV |
| | (SEQ ID NO: 108) | (SEQ ID NO: 118) | (SEQ ID NO: 126) |
| 1A2 | NIGSKG | DDS | QVWDSSSDHWV |
| | (SEQ ID NO: 26) | (SEQ ID NO: 35) | (SEQ ID NO: 37) |
| 1A3 | NIGSKS | DDS | QVWDSSSDHWV |
| | (SEQ ID NO: 109) | (SEQ ID NO: 35) | (SEQ ID NO: 37) |
| 1A6 | NIGSKG | DDS | QVWDSSSDHWV |
| | (SEQ ID NO: 26) | (SEQ ID NO: 35) | (SEQ ID NO: 37) |
| 1B4 | SSNIGSNY | RNN | AAWDDSLNGLV |
| | (SEQ ID NO: 110) | (SEQ ID NO: 119) | (SEQ ID NO: 127) |
| 1C1 | SGSIASNY | EDN | QSYDGITVI |
| | (SEQ ID NO: 111) | (SEQ ID NO: 20) | (SEQ ID NO: 128) |
| 1C4 | SGSIASNY | ADN | QSYDSSNHWV |
| | (SEQ ID NO: 111) | (SEQ ID NO: 120) | (SEQ ID NO: 129) |
| 1C6 | NIGSKS | DDS | QVWDSSSDHWV |
| | (SEQ ID NO: 109) | (SEQ ID NO: 35) | (SEQ ID NO: 37) |
| 1D1 | NIGSKG | DDS | QVWDSRSDHWV |
| | (SEQ ID NO: 26) | (SEQ ID NO: 35) | (SEQ ID NO: 130) |
| 1D2 | RSNIGSNY | SNN | AVWDDSLSGVV |
| | (SEQ ID NO: 112) | (SEQ ID NO: 121) | (SEQ ID NO: 131) |
| 1D4 | SGINVGTYR | YKSDSDK | MIWHSSAYV |
| | (SEQ ID NO: 113) | (SEQ ID NO: 122) | (SEQ ID NO: 132) |
| 1E1 | NIGGKS | DDR | QVWDSSSDHWV |
| | (SEQ ID NO: 114) | (SEQ ID NO: 28) | (SEQ ID NO: 37) |
| 1F1 | NIGSKG | DDR | QVWDSSSDHWV |
| | (SEQ ID NO: 26) | (SEQ ID NO: 28) | (SEQ ID NO: 37) |
| 1G1 | NIGGRV | DDT | QVWDSRSDHPV |
| | (SEQ ID NO: 115) | (SEQ ID NO: 123) | (SEQ ID NO: 133) |
| 1H2 | SLRSYY | GKN | NSRDISDNQWQWI |
| | (SEQ ID NO: 116) | (SEQ ID NO: 124) | (SEQ ID NO: 134) |
| 1H5 | SGSIASHF | GDD | QSYDSSNHVV |
| | (SEQ ID NO: 117) | (SEQ ID NO: 125) | (SEQ ID NO: 135) |

The amino acid sequences of the heavy and light chain framework regions of the PDL-1 antibodies are shown in Table 7A-B below:

TABLE 7A
Heavy chain (V_{H}) framework regions (FRs) of the PDL-1 antibodies

| Sequence | | | | |
|---|---|---|---|---|
| ID | V_{H} FR1 | V_{H} FR2 | V_{H} FR3 | V_{H} FR4 |
| 40 mut | QVQLVQSGAEVK | ISWVRQAPGQGL | NYAQKFQGRVTITADK | WGPGTLVTVSS |
| | KPGSSVKVSCKAS | EWMGG | STSTAYMELSSLRSED | (SEQ ID NO: 7) |
| | (SEQ ID NO: 1) | (SEQ ID NO: 3) | TAVYYC | |
| | | | (SEQ ID NO: 5) | |
| 50-6B6.1 | QVQLVQSGGEVK | ITWVRQAPGQGL | SNAQKVEDRVTMTTD | WGQGTLVTVSS |
| mut | KPGASVKVSCKAS | EWMGWGYTLSS | TSTNTAYMELRSLTAD | (SEQ ID NO: 15) |
| | (SEQ ID NO: 9) | HG | DTAVYYC | |
| | | (SEQ ID NO: 11) | (SEQ ID NO: 13) | |
| 50-6B6.2 | QVQLVQSGGEVK | ITWVRQAPGQGL | SNAQKVEDRVTMTTD | WGQGTLVTVSS |
| | KPGASVKVSCKAS | EWMGWGYTLSS | TSTNTAYMELRSLTAD | (SEQ ID NO: 15) |
| | (SEQ ID NO: 9) | HG | DTAVYYC | |
| | | (SEQ ID NO: 11) | (SEQ ID NO: 13) | |
| 50-7B3 | QVQLVQSGGEVK | ITWVRQAPGQGL | SNAQKVEDRVTMTTD | WGQGTLVTVSS |
| | KPGASVKVSCKAS | EWMGWGYTLSS | TSTNTAYMELRSLTAD | (SEQ ID NO: 15) |
| | (SEQ ID NO: 9) | HG | DTAVYYC | |
| | | (SEQ ID NO: 11) | (SEQ ID NO: 13) | |
| 50-5B9 | QVQLVQSGGEVK | ITWVRQAPGQGL | SNAQKVEDRVTMTTD | WGQGTLVTVSS |
| | KPGASVKVSCKAS | EWMGWGYTLSS | TSTNTAYMELRSLTAD | (SEQ ID NO: 15) |
| | (SEQ ID NO: 9) | HG | DTAVYYC | |
| | | (SEQ ID NO: 11) | (SEQ ID NO: 13) | |
| 14C61 | EVQLVQSGGEVK | ITWVRQAPGQGL | SNAQKVEDRVTMTTD | WGQGTLVTVSS |
| | KPGASVKVSCKAS | EWMGW | TSTNTAYMELRSLTPD | (SEQ ID NO: 15) |
| | (SEQ ID NO: 136) | (SEQ ID NO: 58) | DTAVYYC | |
| | | | (SEQ ID NO: 151) | |
| 1A2 | QVQLVQSGGEVK | ITWVRQAPGQGL | SNAQKVEDRVTMTTD | WGQGTTVTVSS |
| | KPGASVKVSCKAS | EWMGW | TSTNTAYMELRSLTAD | (SEQ ID NO: 160) |
| | (SEQ ID NO: 9) | (SEQ ID NO: 58) | DTAVYYC | |
| | | | (SEQ ID NO: 13) | |
| 1A3 | EVQLVQSGAEVKK | ITWVRQAPGQGL | SNAQKVEDRVTMTTD | WGQGTLVTVSS |
| | PGASVKVSCKAS | EWMGW | TSTNTAYMELRSLTPD | (SEQ ID NO: 15) |
| | (SEQ ID NO: 138) | (SEQ ID NO: 58) | DTAVYYC | |
| | | | (SEQ ID NO: 151) | |
| 1A6 | QVQLVQSGGEVK | ITWVRQAPGQGL | SNAQKVEDRVTMTTD | WGQGTLVTVSS |
| | KPGASVKVSCKAS | EWMGW | TSTNTAYMELRSLTAD | (SEQ ID NO: 15) |
| | (SEQ ID NO: 9) | (SEQ ID NO: 58) | DTAVYYC | |
| | | | (SEQ ID NO: 13) | |
| 1B4 | QVQLVQSGGGLV | MNWVRQAPGKG | DYAAPVKDRFIISRDD | WGEGTTVTVSS |
| | KPGGSLRLSCVGS | LEWVGR | SKNTLYLEMNSLKTED | (SEQ ID NO: 161) |
| | (SEQ ID NO: 139) | (SEQ ID NO: 146) | TGVYYC | |
| | | | (SEQ ID NO: 152) | |
| 1C1 | QVQLVQSGAEVK | ISWVRQAPGQGL | AFAQILEGRVTMTTDT | WGQGTLVTVSS |
| | KPGASVKVSCKAS | EWMGW | STNTAYMELRNLTFDD | (SEQ ID NO: 15) |
| | (SEQ ID NO: 140) | (SEQ ID NO: 147) | TAVYFC | |
| | | | (SEQ ID NO: 153) | |
| 1C4 | QVQLVQSGAEVK | LTWVRQAPGQGL | NYAQKFQGRVTITADK | WGQGTMVTVSS |
| | KPGSSVKVSCKAS | EWMGG | STSTAYMELGSLTSDD | (SEQ ID NO: 162) |
| | (SEQ ID NO: 141) | (SEQ ID NO: 148) | TAVYYC | |
| | | | (SEQ ID NO: 154) | |
| 1C6 | QVQLVQSGGEVK | ITWVRQAPGQGL | SNAQKVEDRVTMTTD | WGQGTLVTVSS |
| | KPGASVKVSCKAS | EWMGW | TSTNTAYMELRSLTPD | (SEQ ID NO: 15) |
| | (SEQ ID NO: 9) | (SEQ ID NO: 58) | DTAVYYC | |
| | | | (SEQ ID NO: 151) | |
| 1D1 | QVQLVQSGAEVK | ISWVRQAPGQGL | SNAQKVEDRVTMTTD | WGQGTTVTVSS |
| | KPGASVKVSCKAS | EWMGW | TSTNTAYMELRNLTTD | (SEQ ID NO: 160) |
| | (SEQ ID NO: 140) | (SEQ ID NO: 147) | DTAVYYC | |
| | | | (SEQ ID NO: 155) | |
| 1D2 | QVQLVQSGAEVK | ISWVRQAPGQGL | NYAQKFQGRVTITADE | WGQGTLVTVSS |
| | KPGSSVKVSCKAS | EWMGG | STSTAYMELSSLRSED | (SEQ ID NO: 15) |
| | (SEQ ID NO: 141) | (SEQ ID NO: 3) | TAVYYC | |
| | | | (SEQ ID NO: 156) | |
| 1D4 | QVQLVQSGGGVV | IHWVRQAPGKGL | YYADSVKGRFTISRDN | WGQGTLVTVSS |
| | QPGRSLRLSCAAS | EWITT | PKNTLYLQMNSLRAED | (SEQ ID NO: 15) |
| | (SEQ ID NO: 142) | (SEQ ID NO: 137) | TAVYYC | |
| | | | (SEQ ID NO: 157) | |
| 1E1 | QVQLVQSGVEMK | ISWVRQAPGQGL | SNAQKLEDRVTMTTD | WGQGTTVTVSS |
| | KPGASVRVSCKGS | EWMGW | TSTNTAYMELRSLTSD | (SEQ ID NO: 160) |
| | (SEQ ID NO: 143) | (SEQ ID NO: 147) | DTAVYYC | |
| | | | (SEQ ID NO: 158) | |
| 1F1 | QVQLVQSGGEVK | ITWVRQAPGQGL | SNAQKVEDRVTMTTD | WGQGTLVTVSS |
| | KPGASVKVSCKAS | EWMGW | TSTNTAYMELRSLTPD | (SEQ ID NO: 15) |
| | (SEQ ID NO: 9) | (SEQ ID NO: 58) | DTAVYYC | |
| | | | (SEQ ID NO: 151) | |
| 1G1 | EVQLVQSGAEVKK | ISWVRQAPGQGL | NYAQKFQGRVTITADK | WGQGTTVTVSS |
| | PGSSVRVSCKAS | EWMGR | STSTAYMELSSLRSED | (SEQ ID NO: 160) |
| | (SEQ ID NO: 144) | (SEQ ID NO: 149) | TAVYYC | |
| | | | (SEQ ID NO: 5) | |
| 1H2 | QVQLVQSGGGLV | MNWVRQAPGKG | TYADSVKGRFTISRDN | WGQGTLVTVSS |
| | KPGGSLRLSCAAS | LVWISR | AKNTLYLQMNSLRAED | (SEQ ID NO: 15) |
| | (SEQ ID NO: 145) | (SEQ ID NO: 150) | TGVYYC | |
| | | | (SEQ ID NO: 159) | |
| 1H5 | QVQLVQSGAEVK | LTWVRQAPGQGL | NYAQKFQGRVTITADK | WGQGTMVTVSS |
| | KPGSSVKVSCKAS | EWMGG | STSTAYMELGSLTSDD | (SEQ ID NO: 162) |
| | (SEQ ID NO: 141) | (SEQ ID NO: 148) | TAVYYC | |
| | | | (SEQ ID NO: 154) | |

TABLE 7B
Heavy chain (V_{L}) framework regions (FRs) of the PDL-1 antibodies

| Sequence | | | | |
|---|---|---|---|---|
| ID | V_{L} FR1 | V_{L} FR2 | V_{L} FR3 | V_{L} FR4 |
| 40 mut | NFMLTQPHSVSESP | VQWYQQRPGSAP | QRPSGVPDRFSGSID | FGGGTKLTVL |
| | GKTVTISCTRS | TTVIY | SSSNSASLTISGLKTE | (SEQ ID NO: 23) |
| | (SEQ ID NO: 17) | (SEQ ID NO: 19) | DEADYYC | |
| | | | (SEQ ID NO: 21) | |
| 50-6B6.1 | SYELTQPPSVSLAP | VHWYQQKPGQAP | DRPSGIPERFSGSNS | FGGGTKLTVL |
| mut | GQSARISCGGD | VVVVY | GNTATLTISRVEAGD | (SEQ ID NO: 23) |
| | (SEQ ID NO: 25) | (SEQ ID NO: 27) | EADYYC | |
| | | | (SEQ ID NO: 29) | |
| 50-6B6.2 | LPVLTQPPSVSAAP | VHWYQQKPGQAP | DRPSGIPERFSGSNS | FGGGTKLTVL |
| | GQTARISCGGS | VLVIY | GNTATLTISRVEAGD | (SEQ ID NO: 23) |
| | (SEQ ID NO: 32) | (SEQ ID NO: 34) | EADYYC | |
| | | | (SEQ ID NO: 29) | |
| 50-7B3 | SYELTQPPSVSVAP | VHWYQQKPGQAP | DRPSGIPERFSGSNS | FGGGTKLTVL |
| | GQTARITCGGN | VLVVY | GNTATLTISRVEAGD | (SEQ ID NO: 23) |
| | (SEQ ID NO: 39) | (SEQ ID NO: 41) | EADYYC | |
| | | | (SEQ ID NO: 29) | |
| 50-5B9 | LPVLTQPPSVSVAL | VHWYQQKPGQAP | SRRSGIPERFSGSHS | FGGGTKLTVL |
| | GQTARITCRGN | VLVVY | GSAATLTISRVEAGD | (SEQ ID NO: 23) |
| | (SEQ ID NO: 43) | (SEQ ID NO: 41) | EADYYC | |
| | | | (SEQ ID NO: 36) | |
| 14C61 | SYELTQPPSVSVAP | VNWYQQRAGQA | ERPSGITVRYSGSNS | FGGGTKLTVL |
| | RQTAKITCTRD | PAVIVY | GNTATLTISRVEAGD | (SEQ ID NO: 23) |
| | (SEQ ID NO: 163) | (SEQ ID NO: 176) | EADYYC | |
| | | | (SEQ ID NO: 187) | |
| 1A2 | QSVLTQPPSVSVA | VHWYQQKPGQAP | DRPSGIPERFSGSNS | FGGGTKLTVL |
| | PGQTARITCGGN | VLVVY | GNTATLTISRVEAGD | (SEQ ID NO: 23) |
| | (SEQ ID NO: 164) | (SEQ ID NO: 41) | EADYYC | |
| | | | (SEQ ID NO: 29) | |
| 1A3 | LPVLTQPPSVSVAP | VHWYQQKPGQAP | DRPSGIPERFSGSNS | FGGGTKLTVL |
| | GQTARITCGGN | VLVVY | GNTATLTISRVEAGD | (SEQ ID NO: 23) |
| | (SEQ ID NO: 165) | (SEQ ID NO: 41) | EADYYC | |
| | | | (SEQ ID NO: 29) | |
| 1A6 | QSVLTQPPSVSVA | VHWYQQKPGQAP | DRPSGIPERFSGSNS | FGGGTKLTVL |
| | PGQTARITCGGN | VLVVY | GNTATLTISRVEAGD | (SEQ ID NO: 23) |
| | (SEQ ID NO: 164) | (SEQ ID NO: 41) | EADYYC | |
| | | | (SEQ ID NO: 29) | |
| 1B4 | LPVLTQPPSASGTP | VYWYQQLPGTAP | QRPSGVPDRFSGSKS | FGTGTRVTVL |
| | GQRVTISCSGS | KLLIY | GTSASLAISGLRSEDE | (SEQ ID NO: 198) |
| | (SEQ ID NO: 166) | (SEQ ID NO: 177) | ADYYC | |
| | | | (SEQ ID NO: 188) | |
| 1C1 | NFMLTQPHSVSES | VQWYQQRPGSSP | QRPSGVPDRFSGSID | FGGGTKLTVL |
| | PGKTVTISCTRS | TTVIY | TSSNSASLTISGLKTK | (SEQ ID NO: 23) |
| | (SEQ ID NO: 167) | (SEQ ID NO: 178) | DEADYYC | |
| | | | (SEQ ID NO: 189) | |
| 1C4 | NFMLTQPHSVSES | VQWYQQRPGSAP | QRPSGVPARFSGSID | FGGGTQLTVL |
| | PGKTVTISCTRS | TTVIF | SSSNSASLTISGLKTE | (SEQ ID NO: 199) |
| | (SEQ ID NO: 167) | (SEQ ID NO: 179) | DEADYYC | |
| | | | (SEQ ID NO: 190) | |
| 1C6 | LPVLTQPPSVSVAP | VHWYQQKPGQAP | DRPSGIPERFSGSNS | FGGGTKLTVL |
| | GQTARITCGGN | VLVVY | GNTATLTINRVEAGD | (SEQ ID NO: 23) |
| | (SEQ ID NO: 165) | (SEQ ID NO: 41) | EADYYC | |
| | | | (SEQ ID NO: 191) | |
| 1D1 | SYELTQPPSVSVAP | VHWYQQTPGQAP | DRPSGIPERFSGSKS | FGGGTRVTVL |
| | GQTARITCGGD | VLVVY | GNTATLTISRVEAGD | (SEQ ID NO: 200) |
| | (SEQ ID NO: 168) | (SEQ ID NO: 180) | EADYYC | |
| | | | (SEQ ID NO: 192) | |
| 1D2 | QPGLTQPPSASGT | VYWYQQFPGTAP | QRPSGVPDRFSGSKS | FGGGTKLTVL |
| | PGQTVTISCSGS | KLLIF | GTSASLAISGLRSEDE | (SEQ ID NO: 23) |
| | (SEQ ID NO: 169) | (SEQ ID NO: 181) | ADYYC | |
| | | | (SEQ ID NO: 188) | |
| 1D4 | LPVLTQPSSLSASP | IYWYQQKPGSPPQ | QQGSGVPSRFSGSK | FGTGTKVTVL |
| | GASASLTCTLR | YLLR | DASANAGILLISGLQS | (SEQ ID NO: 201) |
| | (SEQ ID NO: 170) | (SEQ ID NO: 182) | EDEADYYC | |
| | | | (SEQ ID NO: 193) | |
| 1E1 | SYELTQPPSVSVAP | VHWYQQKPGQAP | DRPSGIPERFSGSNS | FGGGTKLTVL |
| | GQTARIPCGAN | VLVVY | GNTATLTISRVEAGD | (SEQ ID NO: 23) |
| | (SEQ ID NO: 171) | (SEQ ID NO: 41) | EADYYC | |
| | | | (SEQ ID NO: 29) | |
| 1F1 | QPVLTQPPSVSVA | VHWLQQKPGQAP | DRPSGIPERFSGSNS | FGGGTKLTVL |
| | PGQTARITCGGD | VLVVY | GSTATLTISRVEAGD | (SEQ ID NO: 23) |
| | (SEQ ID NO: 172) | (SEQ ID NO: 183) | EADYYC | |
| | | | (SEQ ID NO: 194) | |
| 1G1 | QSVLTQPPSVSVA | VHWYQQRPGQA | DRPSGIPERFSGSNS | FGGGTTLTVL |
| | PGKAANLNCGGK | PVLVIY | GNTATLTITDVEVGD | (SEQ ID NO: 202) |
| | (SEQ ID NO: 173) | (SEQ ID NO: 184) | EADYYC | |
| | | | (SEQ ID NO: 195) | |
| 1H2 | SSELTQDPAVSVAL | ASWYQQKPGQAP | NRPSGIPDRFSGSTS | FGGGTKLAVL |
| | GQTVRITCQGD | VLVIY | GNTASLTITGAQAED | (SEQ ID NO: 203) |
| | (SEQ ID NO: 174) | (SEQ ID NO: 185) | EADYYC | |
| | | | (SEQ ID NO: 196) | |
| 1H5 | NFMLTQPHSVSGS | VQWYQQRPGSSP | QRPSGVPDRISGSID | FGGGTKLTVL |
| | PGETVTISCTRS | TTVIF | TSSNSASLSISGLKTE | (SEQ ID NO: 23) |
| | (SEQ ID NO: 175) | (SEQ ID NO: 186) | DEADYYC | |
| | | | (SEQ ID NO: 197) | |

The PD-L1 antibodies described herein bind to PD-L1. In one embodiment, the PD-L1 antibodies have high affinity and high specificity for PD-L1. Some embodiments also feature antibodies that have a specified percentage identity or similarity to the amino acid or nucleotide sequences of the anti-PD-L1 antibodies described herein. For example, “homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences. For example, the antibodies can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the anti-PD-L1 antibodies described herein. For example, the antibodies can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the anti-PD-L1 antibodies described herein. Sequence identity or similarity to the nucleic acids and proteins of the present invention can be determined by sequence comparison and/or alignment by methods known in the art, for example, using software programs known in the art, such as those described in Ausubel et al. eds. (2007) Current Protocols in Molecular Biology. For example, sequence comparison algorithms (i.e. BLAST or BLAST 2.0), manual alignment or visual inspection can be utilized to determine percent sequence identity or similarity for the nucleic acids and proteins of the present invention.

“Polypeptide” as used herein can encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology, but is not necessarily translated from a designated nucleic acid sequence. It may be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In some embodiments the alteration results in the substitution of an amino acid with a chemically similar amino acid. Conservative substitution tables providing functionally similar amino acids are well known in the art.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

### Antibodies

As used herein, an “antibody” or “antigen-binding polypeptide” can refer to a polypeptide or a polypeptide complex that specifically recognizes and binds to an antigen. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the desired antigen and does not react with other polypeptides.

The terms “antibody fragment” or “antigen-binding fragment”, as used herein, is a portion of an antibody such as F_{(ab′)2}, F_{(ab)2}, F_{ab′}, F_{ab}, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab' and F(ab′)₂, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” refers to a fusion protein of the variable regions of the heavy (V_{H}) and light chains (V_{L}) of immunoglobulins. A single chain Fv (“scFv”) polypeptide molecule is a covalently linked VH:V_{L} heterodimer, which can be expressed from a gene fusion including VH- and VL-encoding genes linked by a peptide-encoding linker. (See Huston et al. (1988) Proc Nat Acad Sci USA 85(16):5879-5883). In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Very large naive human scFv libraries have been and can be created to offer a large source of rearranged antibody genes against a plethora of target molecules. Smaller libraries can be constructed from individuals with infectious diseases in order to isolate disease-specific antibodies. (See Barbas et al., Proc. Natl. Acad. Sci. USA 89:9339-43 (1992); Zebedee et al, Proc. Natl. Acad. Sci. USA 89:3 175-79 (1992)).

Antibody molecules obtained from humans fall into five classes of immunoglubulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (γ, μ, α, δ, ε) with some subclasses among them (e.g., γ1-γ4). Certain classes have subclasses as well, such as IgG₁, IgG₂, IgG₃ and IgG₄ and others. The immunoglobulin subclasses (isotypes) e.g., IgG₁, IgG₂, IgG₃, IgG₄, IgG₅, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” VH and V_{L} regions, which contain the CDRs, as well as frameworks (FRs) of the PD-1 antibodies are shown in Table 1A-Table 15B.

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less inter-molecular variability. The framework regions largely adopt a (β-sheet conformation and the CDRs form loops which connect, and in some cases form part of, the (β-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J. Mol. Biol., 196:901-917 (1987)).

Where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| CDR | Kabat Numbering | Chothia Numbering |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR- H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

As used herein, the term “epitope” can include any protein determinant capable of specific binding to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants usually consist of chemically active surface groupings of molecules such as amino acids or sugar side chains and usually have specific three-dimensional structural characteristics, as well as specific charge characteristics. For example, antibodies can be raised against N- terminal or C-terminal peptides of a polypeptide. More specifically, the antigen-binding site is defined by three CDRs on each of the VH and VL chains (i.e. CDR-H1, CDR-H2, CDR-H3, CDR-L1, CDR-L2 and CDR-L3). In one embodiment, the antibodies can be directed to PD-L1 (having Genbank accession no. NP 054862; 290 amino acid residues in length), comprising the amino acid sequence of SEQ ID NO: 204:

| | |
|---|---|
| 1 | MRIFAVFIFM TYWHLLNAFT VTVPKDLYVV EYGSNMTIEC |
| | KFPVEKQLDL AALIVYWEME |
| 61 | DKNIIQFVHG EEDLKVQHSS YRQRARLLKD QLSLGNAALQ |
| | ITDVKLQDAG VYRCMISYGG |
| 121 | ADYKRITVKV NAPYNKINQR ILVVDPVTSE HELTCQAEGY |
| | PKAEVIWTSS DHQVLSGKTT |
| 181 | TTNSKREEKL FNVTSTLRIN TTTNEIFYCT FRRLDPEENH |
| | TAELVIPELP LAHPPNERTH |
| 241 | LVILGAILLC LGVALTFIFR LRKGRMMDVK KCGIQDTNSK |
| | KQSDTHLEET |

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the equilibrium binding constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361: 186-87 (1993)). The ratio of K_{off}/Kₒₙ enables the cancellation of all parameters not related to affinity, and is equal to the equilibrium binding constant, K_{D}. (See, generally, Davies et al. (1990) Annual Rev Biochem 59:439-473). An antibody of the present invention can specifically bind to a PD-1 epitope when the equilibrium binding constant (K_{D}) is ≤1 μM, ≤10 μM, ≤10 nM, ≤10 pM, or <100 pM to about 1 pM, as measured by kinetic assays such as radioligand binding assays or similar assays known to those skilled in the art, such as BIAcore or Octet (BLI). For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a K_{D} about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1E-7 M and a K_{D} about 1E-6 M. For example, in some embodiments, the K_{D} is about 1E-12 M while in other embodiments the K_{D} is about 1E-11 M. In some embodiments, the K_{D} is about 1E-10 M while in other embodiments the K_{D} is about 1E-9 M. In some embodiments, the K_{D} is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3 E-11 M, while in other embodiments the K_{D} is about 3E-12 M. In some embodiments, the K_{D} is about 6E-11 M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it would bind to a random, unrelated epitope.

For example, the PD-L1 antibody can be monovalent or bivalent, and comprises a single or double chain. Functionally, the binding affinity of the PD-L1 antibody is within the range of 10⁻⁵M to 10⁻¹²M. For example, the binding affinity of the PD-L1 antibody is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷ M to 10⁻¹¹ M, from 10⁻⁸ M to 10 ⁻¹¹ M, from 10⁻⁹M to 10⁻¹¹M, from 10⁻¹⁰ M to 10⁻¹¹ M, from 10⁻⁵ M to 10⁻¹⁰ M, from 10⁻⁶ M to 10⁻¹⁰ M, from 10⁻⁷ M to 10⁻¹⁰ M, from 10⁻⁸ M to 10⁻¹⁰ M, from 10⁻⁹ M to 10⁻¹⁰ M, from 10⁻⁵M to 10⁻⁹M, from 10⁻⁶M to 10⁻⁹M, from 10⁻⁷M to 10⁻⁹M, from 10⁻⁸M to 10⁻⁹M, from 10⁻⁵M to 10⁻⁸M, from 10⁻⁶M to 10⁻⁸M, from 10⁻⁷M to 10⁻⁸M, from 10⁻⁵M to 10⁻⁷ M, from 10⁻⁶ M to 10⁻⁷ M, or from 10⁻⁵ M to 10⁻⁶ M.

A PD-L1 protein of the invention, or a derivative, fragment, analog, homolog or ortholog thereof, can be utilized as an immunogen in the generation of antibodies that immunospecifically bind these protein components, e.g., amino acid residues comprising SEQ ID NO: 204. A PD-L1 protein or a derivative, fragment, analog, homolog, or ortholog thereof, coupled to a proteoliposome can be utilized as an immunogen in the generation of antibodies that immunospecifically bind these protein components.

Those skilled in the art will recognize that it is possible to determine, without undue experimentation, if a human monoclonal antibody has the same specificity as a human monoclonal antibody of the invention by ascertaining whether the former prevents the latter from binding to PD-L1. For example, if the human monoclonal antibody being tested competes with the human monoclonal antibody of the invention, as shown by a decrease in binding by the human monoclonal antibody of the invention, then it is likely that the two monoclonal antibodies bind to the same, or to a closely related, epitope.

Another way to determine whether a human monoclonal antibody has the specificity of a human monoclonal antibody of the invention is to pre-incubate the human monoclonal antibody of the invention with the PD-L1 protein, with which it is normally reactive, and then add the human monoclonal antibody being tested to determine if the human monoclonal antibody being tested is inhibited in its ability to bind PD-L1. If the human monoclonal antibody being tested is inhibited then, in all likelihood, it has the same, or functionally equivalent, epitopic specificity as the monoclonal antibody of the invention. Screening of human monoclonal antibodies of the invention can be also carried out by utilizing PD-L1 and determining whether the test monoclonal antibody is able to neutralize PD-L1.

Various procedures known within the art can be used for the production of polyclonal or monoclonal antibodies directed against a protein of the invention, or against derivatives, fragments, analogs homologs or orthologs thereof (See, for example, Antibodies: A Laboratory Manual, Harlow E, and Lane D, 1988, Cold Spring Harbor Laboratory Press, Cold Spring Harbor, N.Y., incorporated herein by reference).

Antibodies can be purified by well-known techniques, such as affinity chromatography using protein A or protein G, which provide primarily the IgG fraction of immune serum. Subsequently, or alternatively, the specific antigen, which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with an epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent can include the protein antigen, a fragment thereof or a fusion protein thereof. For example, peripheral blood lymphocytes can be used if cells of human origin are desired, or spleen cells or lymph node cells can be used if non-human mammalian sources are desired. The lymphocytes are then fused with an immortalized cell line using a suitable fusing agent, such as polyethylene glycol, to form a hybridoma cell (See Goding, Monoclonal Antibodies: Principles and Practice, Academic Press, (1986) pp. 59-103). Immortalized cell lines can be transformed mammalian cells, particularly myeloma cells of rodent, bovine and human origin. For example, rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell lines that are useful are those that fuse efficiently, support stable high-level expression of antibody by the selected antibody-producing cells, and are sensitive to a medium such as HAT medium. For example, immortalized cell lines can be murine myeloma lines, which can be obtained, for instance, from the Salk Institute Cell Distribution Center (San Diego, Calif.) and the American Type Culture Collection (Manassas, Va.). Human myeloma and mouse-human heteromyeloma cell lines also have been described for the production of human monoclonal antibodies. (See Kozbor, J. Immunol, 133:3001 (1984); Brodeur et al, Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. For example, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567 (incorporated herein by reference in its entirety). DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (See U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Fully human antibodies are antibody molecules in which the entire sequence of both the light chain and the heavy chain, including the CDRs, arise from human genes. Such antibodies are termed “human antibodies” or “fully human antibodies” herein. Human monoclonal antibodies can be prepared by using trioma technique; the human B-cell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

“Humanized antibodies” can be antibodies from non-human species (such as a mouse) whose light chain and heavy chain protein sequences have been modified to increase their similarity to antibody variants produced in humans. Humanized antibodies are antibody molecules derived from a non-human species antibody that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen-binding. These framework substitutions are identified by methods well known in the art, e.g., by modeling of the interactions of the CDR and framework residues to identify framework residues important for antigen-binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) For example, the non-human part of the antibody (such as the CDR(s) of a light chain and/or heavy chain) can bind to the target antigen.

Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., Proc. Natl. Sci. USA 91:969-973 (1994)), and chain shuffling (U.S. Pat. No. 5,565,332, which is incorporated by reference in its entirety). “Humanization” (also called Reshaping or CDR-grafting) is a well-established technique understood by the skilled artisan for reducing the immunogenicity of monoclonal antibodies (mAbs) from xenogeneic sources (commonly rodent) and for improving their activation of the human immune system (See, for example, Hou S, Li B, Wang L, Qian W, Zhang D, Hong X, Wang H, Guo Y (July 2008). “Humanization of an anti-CD34 monoclonal antibody by complementarity-determining region grafting based on computer-assisted molecular modeling”. J Biochem. 144 (1): 115-20). Antibodies can be humanized by methods known in the art, such as CDR-grafting. See also, Safdari et al., (2013) Biotechnol Genet Eng Rev.; 29:175-86. In addition, humanized antibodies can be produced in transgenic plants, as an inexpensive production alternative to existing mammalian systems. For example, the transgenic plant may be a tobacco plant, i.e., Nicotiania benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can be readily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the present invention further provides any cell or plant comprising a vector that encodes the antibody of the present invention, or produces the antibody of the present invention.

Human monoclonal antibodies, such as fully human and humanized antibodies, can be prepared by using trioma technique; the human B-cell hybridoma technique (see Kozbor, et al, 1983 Immunol Today 4: 72); and the EBV hybridoma technique to produce human monoclonal antibodies (see Cole, et al, 1985 In: MONOCLONAL ANTIBODIES AND CANCER THERAPY, Alan R. Liss, Inc., pp. 77-96). Human monoclonal antibodies can be utilized and can be produced by using human hybridomas (see Cote, et al, 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using other techniques, including phage display libraries. (See Hoogenboom and Winter, J. Mol. Biol, 227:381 (1991); Marks et al., J. Mol. Biol, 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al., Bio/Technology 10, 779-783 (1992); Lonberg et al, Nature 368, 856-859 (1994); Morrison, Nature 368, 812-13 (1994); Fishwild et al, Nature Biotechnology 14, 845-51 (1996); Neuberger, Nature Biotechnology 14, 826 (1996); and Lonberg and Huszar, Intern. Rev. Immunol. 13 65-93 (1995).

Human antibodies can additionally be produced using transgenic nonhuman animals which are modified so as to produce fully human antibodies rather than the animal's endogenous antibodies in response to challenge by an antigen. (See, PCT publication no. WO94/02602 and U.S. Pat. No. 6,673,986). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. A non-limiting example of such a nonhuman animal is a mouse, and is termed the Xenomouse™ as disclosed in PCT publication nos. WO96/33735 and WO96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv (scFv) molecules.

Thus, using such a technique, therapeutically useful IgG, IgA, IgM and IgE antibodies can be produced. For an overview of this technology for producing human antibodies, see Lonberg and Huszar Int. Rev. Immunol. 73:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Creative BioLabs (Shirley, N.Y.) can be engaged to provide human antibodies directed against a selected antigen using technology similar to that described herein.

An example of a method of producing a nonhuman host, exemplified as a mouse, lacking expression of an endogenous immunoglobulin heavy chain is disclosed in U.S. Pat. No. 5,939,598. It can be obtained by a method, which includes deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, is disclosed in PCT publication No. WO99/53049.

The antibody of interest can also be expressed by a vector containing a DNA segment encoding the single chain antibody described herein.

These vectors can include liposomes, naked DNA, adjuvant-assisted DNA, gene gun, catheters, etc. Vectors can further include chemical conjugates such as described in WO 93/64701, which has targeting moiety (e.g. a ligand to a cellular surface receptor), and a nucleic acid binding moiety (e.g. polylysine), viral vectors (e.g. a DNA or RNA viral vector), fusion proteins such as described in PCT/US 95/02140 (WO 95/22618), which is a fusion protein containing a target moiety (e.g. an antibody specific for a target cell) and a nucleic acid binding moiety (e.g. a protamine), plasmids, phage, viral vectors, etc. The vectors can be chromosomal, non-chromosomal or synthetic. Retroviral vectors can also be used, and include moloney murine leukemia viruses.

DNA viral vectors can also be used, and include pox vectors such as orthopox or avipox vectors, herpesvirus vectors such as a herpes simplex I virus (HSV) vector (See Geller, A. I. et al, J. Neurochem, 64:487 (1995); Lim, F., et al, in DNA Cloning: Mammalian Systems, D. Glover, Ed. (Oxford Univ. Press, Oxford England) (1995); Geller, A. I. et al, Proc Natl. Acad. Sci.: U.S.A. 90:7603 (1993); Geller, A. I., et al, Proc Natl. Acad. Sci USA 87: 1149 (1990), Adenovirus Vectors (see LeGal LaSalle et al, Science, 259:988 (1993); Davidson, et al, Nat. Genet 3: 219 (1993); Yang, et al, J. Virol. 69:2004 (1995) and Adeno-associated Virus Vectors (see Kaplitt, M. G.. et al, Nat. Genet. 8: 148 (1994).

Pox viral vectors introduce the gene into the cell's cytoplasm. Avipox virus vectors result in only a short-term expression of the nucleic acid. Adenovirus vectors, adeno-associated virus vectors, and herpes simplex virus (HSV) vectors can be used for introducing the nucleic acid into neural cells. The adenovirus vector results in a shorter-term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaP0₄ precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g. adenovirus, HSV) to a desired location. Additionally, the particles can be delivered by intracerebroventricular (icy) infusion using a minipump infusion system, such as a SynchroMed Infusion System. A method based on bulk flow, termed convection, has also proven effective at delivering large molecules to extended areas of the brain and can be useful in delivering the vector to the target cell. (See Bobo et al, Proc. Natl. Acad. Sci. USA 91: 2076-2080 (1994); Morrison et al, Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways, for example, to detect the presence of PD-L1 in a sample. The antibody can also be used to try to bind to and disrupt a PD-L1 activity. In an embodiment, the antibodies of the present invention are full-length antibodies, containing an Fc region similar to wild-type Fc regions that bind to Fc receptors.

Techniques can be adapted for the production of single-chain antibodies specific to an antigenic protein of the invention (See e.g., U.S. Pat. No. 4,946,778). In addition, methods can be adapted for the construction of Fab expression libraries (See e.g., Huse, et al, 1989 Science 246: 1275-1281) to allow rapid and effective identification of monoclonal Fab fragments with the desired specificity for a protein or derivatives, fragments, analogs or homologs thereof Antibody fragments that contain the idiotypes to a protein antigen can be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Heteroconjugate antibodies are also within the scope of the present invention. Heteroconjugate antibodies are composed of two covalently joined antibodies. Such antibodies can, for example, target immune system cells to unwanted cells (see U.S. Pat. No. 4,676,980), and for treatment of HIV infection (See PCT Publication Nos. WO91/00360; WP92/20373). The antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

The antibody of the invention can be modified with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al, J. Exp Med., 176: 1 191-1 195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al, Anti-Cancer Drug Design, 3: 219-230 (1989)).

In certain embodiments, an antibody of the invention can comprise an Fc variant comprising an amino acid substitution which alters the antigen-independent effector functions of the antibody, in particular the circulating half-life of the antibody. Such antibodies exhibit either increased or decreased binding to FcRn when compared to antibodies lacking these substitutions, therefore, have an increased or decreased half-life in serum, respectively. Fc variants with improved affinity for FcRn are anticipated to have longer serum half-lives, and such molecules have useful applications in methods of treating mammals where long half-life of the administered antibody is desired, e.g., to treat a chronic disease or disorder. In contrast, Fc variants with decreased FcRn binding affinity are expected to have shorter halt-lives, and such molecules are also useful, for example, for administration to a mammal where a shortened circulation time can be advantageous, e.g., for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity can be desired include those applications in which localization to the brain, kidney, and/or liver is desired. In one embodiment, the Fc variant-containing antibodies can exhibit reduced transport across the epithelium of kidney glomeruli from the vasculature. In another embodiment, the Fc variant-containing antibodies can exhibit reduced transport across the blood brain barrier (BBB) from the brain, into the vascular space. In one embodiment, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05/047327 which is incorporated by reference herein. In certain exemplary embodiments, the antibodies, or fragments thereof, of the invention comprise an Fc domain having one or more of the following substitutions: V284E, H285E, N286D, K290E and S304D (EU numbering).

In some embodiments, mutations are introduced to the constant regions of the mAb such that the antibody dependent cell-mediated cytotoxicity (ADCC) activity of the mAb is altered. For example, the mutation is a LALA mutation in the CH2 domain. In one embodiment, the antibody (e.g., a human mAb, or a bispecific Ab) contains mutations on one scFv unit of the heterodimeric mAb, which reduces the ADCC activity. In another embodiment, the mAb contains mutations on both chains of the heterodimeric mAb, which completely ablates the ADCC activity. For example, the mutations introduced into one or both scFv units of the mAb are LALA mutations in the CH2 domain. These mAbs with variable ADCC activity can be optimized such that the mAbs exhibits maximal selective killing towards cells that express one antigen that is recognized by the mAb, however exhibits minimal killing towards the second antigen that is recognized by the mAb.

In other embodiments, antibodies of the invention for use in the diagnostic and treatment methods described herein have a constant region, e.g., an IgG₁ or IgG₄ heavy chain constant region, which can be altered to reduce or eliminate glycosylation. For example, an antibody of the invention can also comprise an Fc variant comprising an amino acid substitution which alters the glycosylation of the antibody. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS. In one embodiment, the antibody comprises an Fc variant with an amino acid substitution at amino acid position 228 or 299 (EU numbering). In more particular embodiments, the antibody comprises an IgG1 or IgG4 constant region comprising an S228P and a T299A mutation (EU numbering).

Exemplary amino acid substitutions which confer reduced or altered glycosylation are described in PCT Publication No, WO05/018572, which is incorporated by reference herein in its entirety. In some embodiments, the antibodies of the invention, or fragments thereof, are modified to eliminate glycosylation. Such antibodies, or fragments thereof, can be referred to as “agly” antibodies, or fragments thereof, (e.g. “agly” antibodies). While not wishing to be bound by theory “agly” antibodies, or fragments thereof, can have an improved safety and stability profile in vivo. Exemplary agly antibodies, or fragments thereof, comprise an aglycosylated Fc region of an IgG4 antibody which is devoid of Fc-effector function thereby eliminating the potential for Fc mediated toxicity to the normal vital tissues and cells that express PD-L1. In yet other embodiments, antibodies of the invention, or fragments thereof, comprise an altered glycan. For example, the antibody can have a reduced number of fucose residues on an N-glycan at Asn297 of the Fc region, i.e., is afucosylated. In another embodiment, the antibody can have an altered number of sialic acid residues on the N-glycan at Asn297 of the Fc region.

The invention also is directed to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Non-limiting examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al, Science 238: 1098 (1987). Carbon- 14-labeled 1-isothiocyanatobenzyl-3-methyldiethylene triaminepentaacetic acid (MX-DTPA) is an exemplary chelating agent for conjugation of radionucleotide to the antibody. (See PCT Publication No. WO94/11026, and U.S. Pat. No. 5,736,137).

Those of ordinary skill in the art understand that a large variety of possible moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding, and complexation. In one embodiment, binding is, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133: 1335-2549 (1984); Jansen et al., Immunological Reviews 62: 185-216 (1982); and Vitetta et al, Science 238: 1098 (1987)). Non-limiting examples of linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Non-limiting examples of useful linkers that can be used with the antibodies of the invention include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (-hydroxysulfo-succinimide: Pierce Chem. Co., Cat. #24510) conjugated to EDC.

The linkers described herein contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al, Proc. Natl. Acad. Sci. USA, 82: 3688 (1985); Hwang et al, Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Non-limiting examples of useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al, J. Biol. Chem., 257: 286-288 (1982) via a disulfide-interchange reaction.

### Multispecific Antibodies

Multispecific antibodies are antibodies that can recognize two or more different antigens. For example, a bi-specific antibody (bsAb) is an antibody comprising two variable domains or scFv units such that the resulting antibody recognizes two different antigens. For example, a trispecific antibody (tsAb) is an antibody comprising two variable domains or scFv units such that the resulting antibody recognizes three different antigens. The present invention provides for multispecific antibodies, such as bi-specific antibodies that recognize PD-L1 and a second antigen. For example, PD-L1 is an immune checkpoint molecule and is also a tumor antigen. As a tumor antigen targeting molecule, an antibody or antigen-binding fragment specific to PD-L1 can be combined with a second antigen-binding fragment specific to an immune cell to generate a bispecific antibody. In some embodiments, the immune cell is selected from the group consisting of a T cell, a B cell, a monocyte, a macrophage, a neutrophil, a dendritic cell, a phagocyte, a natural killer cell, an eosinophil, a basophil, and a mast cell. Molecules on the immune cell which can be targeted include, but not limited to, for example, CD3, CD16, CD19, CD28, and CD64. Other non-limiting examples include PD-1, CTLA-4, LAG-3 (also known as CD223), CD28, CD122, 4-1BB (also known as CD137), TIM3, OX-40 or OX40L, CD40 or CD40L, LIGHT, ICOS/ICOSL, GITR/GITRL, TIGIT, CD27, VISTA, B7H3, B7H4, HEVM or BTLA (also known as CD272), killer-cell immunoglobulin-like receptors (KIRs), and CD47. Exemplary second antigens include tumor associated antigens (e.g., LINGO1, EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP and Tenascin), cytokines (e.g., IL-2, IL-3, IL-4, IL-5, IL-6, IL-7, IL-10, IL-12, IL-13, IL-15, GM-CSF, TNF-α, CD40L, OX40L, CD27L, CD3OL, 4-1BBL, LIGHT and GITRL), and cell surface receptors. Different formats of bispecific antibodies are also provided herein. In some embodiments, each of the anti-PD-L1 fragment and the second fragment is each independently selected from a Fab fragment, a single-chain variable fragment (scFv), or a single-domain antibody. In some embodiments, the bispecific antibody further includes a Fc fragment. A bi-specific antibody of the present invention comprises a heavy chain and a light chain combination or scFv of the PD-L1 antibodies disclosed herein.

For example, the nucleic acid and amino acid sequence of the bispecific PD-L1 antibodies (such as GITR-PD-1L fusions) are provided below, in addition to exemplary constant regions useful in combination with the VH and V_{L} sequences provided herein:

TABLE 11A
Ab #E1-3H7 Variable Region nucleic acid sequences

V_{H} chain of Ab #E1-3H7 VH (IGHV3-23*04)
CAGGTGCAGCTGGTGCAGTCTGGGGGAGGCTTGGTACAGCCTGGGGGGTC
CCTGAGACTCTCCTGTGCAGCCTCTGGATTCACCTTTAGCAGCCATGCCA
TGAGCTGGGTCCGCCAGGCTCCAGGGAAGGGGCTGGAGTGGGTCTCAGCT
ATTAGTGGTAGTGGTGGTAGCACATACTACGCAGACTCCGTGAAGGGCCG
GTTCACCATCTCCAGAGACAATTCCAAGAACACGCTGTATCTGCAAATGA
ACAGCCTGAGAGCCGAGGACACGGCCGTATATTACTGTGCGAAAATCGGT
ACGGCGGATGCTTTTGATATCTGGGGCCAAGGGACCACGGTCACCGTCTC
CTCAG (SEQ ID NO: 211)
V_{L} chain of Ab #E1-3H7 VL (IGLV1-44*01)
CAGTCTGCCCTGACTCAGCCACCCTCAGTGTCTGGGACCCCCGGACAGAG
GGTCACCATCTCTTGTTCTGGAGGCGTCCCCAACATCGGAAGTAATCCTG
TAAACTGGTACCTCCACCGCCCAGGAACGGCCCCCAAACTCCTCATCTAT
AATAGCAATCAGTGGCCCTCAGGGGTCCCTGACCGATTTTCTGGCTCCAG
GTCTGGCACCTCAGCCTCCCTGGCCATCAGTGGGCTCCAGTCTGAGGATG
AGGCTGATTATTACTGTGCAGCATGGGATGACAGCCTGGATGGTCTGGTT
TTCGGCGGAGGGACCAAGTTGACCGTCCTAG (SEQ ID NO: 212)

TABLE 11B
Ab #E1-3H7 Variable Region amino acid sequences

V_{H} chain of Ab #E1-3H7 VH (IGHV3-23*04)
QVQLVQSGGGLVQPGGSLRLSCAASGFTFSSHAMSWVRQAPGKGLEWVSA
ISGSGGSTYYADSVKGRFTISRDNSKNTLYLQMNSLRAEDTAVYYCAKIG
TADAFDIWGQGTTVTVSS (SEQ ID NO: 213)
V_{L} chain of Ab #E1-3H7 VL (IGLV1-44*01)
QSALTQPPSVSGTPGQRVTISCSGGVPNIGSNPVNWYLHRPGTAPKLLIY
NSNQWPSGVPDRFSGSRSGTSASLAISGLQSEDEADYYCAAWDDSLDGLV
FGGGTKLTVL (SEQ ID NO: 214)

TABLE 12A
Ab #E1-3H7 Constant Region nucleic acid
sequences - wild type IgG1 monomer

CH1
ACCAAGGGCCCATCGGTCTTCCCCCTGGCACCCTCCTCCAAGAGCACCTC
TGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACC
TTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGA
ATCACAAGCCCAGCAACACCAAGGTGGACAAGAAA (SEQ ID NO:
215)
Hinge
GCAGAGCCCAAATCTTGTGACAAAACTCACACATGCCCACCGTGCCCA
(SEQ ID NO: 216)
CH2
GCACCTGAACTCCTGGGGGGACCGTCAGTCTTCCTCTTCCCCCCAAAACC
CAAGGACACCCTCATGATCTCCCGGACCCCTGAGGTCACATGCGTGGTGG
TGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGC
TGAATGGCAAGGAGTACAAGTGCAAGGTCTCCAACAAAGCCCTCCCAGCC
CCCATCGAGAAAACCATCTCCAAAGCCAAA (SEQ ID NO: 217)
CH3
GGGCAGCCCCGAGAACCACAGGTGTACACCCTGCCCCCATCCCGGGATGA
GCTGACCAAGAACCAGGTCAGCCTGACCTGCCTGGTCAAAGGCTTCTATC
CCAGCGACATCGCCGTGGAGTGGGAGAGCAATGGGCAGCCGGAGAACAAC
TACAAGACCACGCCTCCCGTGCTGGACTCCGACGGCTCCTTCTTCCTCTA
CAGCAAGCTCACCGTGGACAAGAGCAGGTGGCAGCAGGGGAACGTCTTCT
CATGCTCCGTGATGCATGAGGCTCTGCACAACCACTACACGCAGAAGAGC
CTCTCCCTGTCTCCGGGTAAATGA (SEQ ID NO: 218)
C_{L}
GGTCAGCCCAAGGCTGCCCCCTCGGTCACTCTGTTCCCGCCCTCCTCTGA
GGAGCTTCAAGCCAACAAGGCCACACTGGTGTGTCTCATAAGTGACTTCT
ACCCGGGAGCCGTGACAGTGGCCTGGAAGGCAGATGGCAGCCCCGTCAAG
GCGGGAGTGGAGACCACCACACCCTCCAAACAAAGCAACAACAAGTACGC
GGCCAGCAGCTATCTGAGCCTGACGCCTGAGCAGTGGAAGTCCCACAGAA
GCTACAGCTGCCAGGTCACGCATGAAGGGAGCACCGTGGAGAAGACAGTG
GCCCCTACAGAATGTTCATGA (SEQ ID NO: 219)

TABLE 12B
Ab #E1-3H7 Constant Region amino acid sequences -
wild type IgG1 monomer (same for the anti-CCR4
mAb2.3 construct except C_{L}. Also note that the
aqua highlighted (bolded) residues in CH2 and CH3
are wild type residues to be mutated to make
different IgG1 mutants (yellow highlighted (bolded
and italicized) residues in Tables 13-22 are anti-
PD1 scFvs)

CH1
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKK
(SEQ ID NO: 220)
Hinge
AEPKSCDKTHTCPPCP (SEQ ID NO: 221)
CH2
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK (SEQ ID NO: 222)
CH3
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK (SEQ ID NO: 223)
C_{L}
GQPKAAPSVTLFPPSSEELQANKATLVCLISDFYPGAVTVAWKADGSPVK
AGVETTTPSKQSNNKYAASSYLSLTPEQWKSHRSYSCQVTHEGSTVEKTV
APTECS (SEQ ID NO: 224)

TABLE 13A
Ab #E1-3H7 Constant Region nucleic acid
sequences - IgG1 LALA-aPDL-1 40 mut

CH1
Same as wild type (see Table 12A)
Hinge
Same as wild type (see Table 12A)
CH2 (identical to CH2 in Table 13A)
GCACCTGAA [symbol] GGGGGACCGTCAGTCTTCCTCTTCCCCCCAAAACC
CAAGGACACCCTCATGATCTCCCGGACCCCTGAGGTCACATGCGTGGTGG
TGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGC
TGAATGGCAAGGAGTACAAGTGCAAGGTCTCCAACAAAGCCCTCCCAGCC
CCCATCGAGAAAACCATCTCCAAAGCCAAA (SEQ ID NO: 225)
CH3 (identical to CH3 in Table 14A)
GGGCAGCCCCGA [symbol] CCACAGGTGTACACCCTGCCCCCATCCCGGGATGA
GCTGACCAAGAACCAGGTCAGCCTGACCTGCCTGGTCAAAGGCTTCTATC
CCAGCGACATCGCCGTGGAGTGGGAGAGCAATGGGCAGCCGGAGAACAAC
TACAAGACCACGCCTCCCGTGCTGGACTCCGACGGCTCCTTCTTCCTCTA
CAGCAAGCTCACCGTGGACAAGAGCAGGTGGCAGCAGGGGAACGTCTTCT
CATGCTCCGTGATGCAT [symbol] GCTCTGCACAACCACTACACGCAGAAGAGC
CTCTCCCTGTCTCCGGGTAAATGA (SEQ ID NO: 226)
C_{L} (CL in frame fusion with an scFv such as anti-
PD-L1 40 mut (lower case letters)
GGTCAGCCCAAGGCTGCCCCCTCGGTCACTCTGTTCCCGCCCTCCTCTGA
GGAGCTTCAAGCCAACAAGGCCACACTGGTGTGTCTCATAAGTGACTTCT
ACCCGGGAGCCGTGACAGTGGCCTGGAAGGCAGATGGCAGCCCCGTCAAG
GCGGGAGTGGAGACCACCACACCCTCCAAACAAAGCAACAACAAGTACGC
GGCCAGCAGCTATCTGAGCCTGACGCCTGAGCAGTGGAAGTCCCACAGAA
GCTACAGCTGCCAGGTCACGCATGAAGGGAGCACCGTGGAGAAGACAGTG
GCCCCTACAGAATGTTCAggtggcggcggttccggaggtggtggttcaca
ggtgcagctggtgcagtctggggctgaggtgaagaagcctgggtcctcgg
tgaaggtctcctgcaaggcttctggaggcaccttcagcagctatgctatc
agctgggtgcgacaggcccctggacaagggcttgagtggatgggagggat
catccctatctttggtacagcaaactacgcacagaagttccagggcagag
tcacgattaccgcggacaaatccacgagcacagcctacatggagctgagc
agcctgagatctgaggacacggccgtctattactgtgcgagagggcgtca
aatgttcggtgcgggaattgatttctggggcccgggcaccctggtcaccg
tctcctcaggtggcggcggttccggaggtggtggttctggcggtggtggc
agcatcaattttatgctgactcagccccactctgtgtcggagtctccggg
gaagacggtaaccatctcctgcacccgcagcagtggcagcattgacagca
actatgtgcagtggtaccagcagcgcccgggcagcgcccccaccactgtg
atctatgaggataaccaaagaccctctggggtccctgatcggttctctgg
ctccatcgacagctcctccaactctgcctccctcaccatctctggactga
agactgaggacgaggctgactactactgtcagtcttatgatagcaacaat
cgtcatgtgatattcggcggagggaccaagctgaccgtcctaggt (SEQ
ID NO: 227)

TABLE 13B
Ab #E1-3H7 Constant Region amino acid sequences -
IgG1 LALA-aPDL-1 40 mut

CH1
Same as wild type (see Table 12B)
Hinge
Same as wild type (see Table 12B)
CH2 (identical to CH2 in Table 13B)
APE [symbol] GGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK (SEQ ID NO: 228)
CH3 (identical to CH3 in Table 14B)
GQPR [symbol] PQVYTLPPSRDELTKNQVSLTCLVKGFYPSDIAVEWESNGQPENN
YKTTPPVLDSDGSFFLYSKLTVDKSRWQQGNVFSCSVMH [symbol] ALHNHYTQKS
LSLSPGK (SEQ ID NO: 229)
C_{L} (CL in frame fusion with an scFv such as anti-
PD-L1 40 mut (lowercase letters), underlined
sequences denotes linkers (1) between C_{L} and scFv,
and (2) between V_{H} and V_{L} within the scFv)
GQPKAAPSVTLFPPSSEELQANKATLVCLISDFYPGAVTVAWKADGSPVK
AGVETTTPSKQSNNKYAASSYLSLTPEQWKSHRSYSCQVTHEGSTVEKTV
APTECSggggsggggsqvqlvqsgaevkkpgssvkvsckasggtfssyai
swvrqapgqglewmggiipifgtanyaqkfqgrvtitadkststaymels
slrsedtavyycargrqmfgagidfwgpgtlvtvssggggsggggsgggg
sinfmltqphsvsespgktvtisctrssgsidsnvvqwvqqrpgsapttv
iyednqrpsgvpdrfsgsidsssnsasltisglktedeadyycqsydsnn
rhvifgggtkltvlg (SEQ ID NO: 230)

TABLE 14A
Ab #E1-3H7 Constant Region nucleic acid
sequences - IgG1 LALA-aPD-L1 50-6B6.1 mut

CH1
Same as wild type (see Table 12A)
Hinge
Same as wild type (see Table 12A)
CH2 (identical to CH2 in Table 13A)
GCACCTGAA [symbol] GGGGGACCGTCAGTCTTCCTCTTCCCCCCAAAACC
CAAGGACACCCTCATGATCTCCCGGACCCCTGAGGTCACATGCGTGGTGG
TGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGC
TGAATGGCAAGGAGTACAAGTGCAAGGTCTCCAACAAAGCCCTCCCAGCC
CCCATCGAGAAAACCATCTCCAAAGCCAAA (SEQ ID NO: 225)
CH3 (identical to CH3 in Table 14A)
GGGCAGCCCCGA [symbol] CCACAGGTGTACACCCTGCCCCCATCCCGGGATGA
GCTGACCAAGAACCAGGTCAGCCTGACCTGCCTGGTCAAAGGCTTCTATC
CCAGCGACATCGCCGTGGAGTGGGAGAGCAATGGGCAGCCGGAGAACAAC
TACAAGACCACGCCTCCCGTGCTGGACTCCGACGGCTCCTTCTTCCTCTA
CAGCAAGCTCACCGTGGACAAGAGCAGGTGGCAGCAGGGGAACGTCTTCT
CATGCTCCGTGATGCAT [symbol] GCTCTGCACAACCACTACACGCAGAAGAGC
CTCTCCCTGTCTCCGGGTAAATGA (SEQ ID NO: 226)
C_{L} (CL in frame fusion with anti-PD-L1 scFv 50-
6B6.1 mut(lowercase letters))
GGTCAGCCCAAGGCTGCCCCCTCGGTCACTCTGTTCCCGCCCTCCTCTGA
GGAGCTTCAAGCCAACAAGGCCACACTGGTGTGTCTCATAAGTGACTTCT
ACCCGGGAGCCGTGACAGTGGCCTGGAAGGCAGATGGCAGCCCCGTCAAG
GCGGGAGTGGAGACCACCACACCCTCCAAACAAAGCAACAACAAGTACGC
GGCCAGCAGCTATCTGAGCCTGACGCCTGAGCAGTGGAAGTCCCACAGAA
GCTACAGCTGCCAGGTCACGCATGAAGGGAGCACCGTGGAGAAGACAGTG
GCCCCTACAGAATGTTCAggtggcggeggttccggaggtggtggttcatc
gatggcccaggtgcagctggtgcagtctggaggtgaggtgaagaagccgg
gggcctcagtgaaggtctcctgcaaggcttctggttacaccttgagcagt
catggtataacctgggtgcgacaggcccctggacaagggcttgagtggat
gggatggatcagcgctcacaatggtcacgctagcaatgcacagaaggtgg
aggacagagtcactatgactactgacacatccacgaacacagcctacatg
gaactgaggagcctgacagctgacgacacggccgtgtattactgtgcgag
agtacatgctgccctctactatggtatggacgtctggggccaaggaaccc
tggtcaccgtctcctcaggtggcggcggttccggaggtggtggtgctggc
ggtggtggcagctcctatgagctgactcagccaccctcggtgtcactggc
cccaggacagtcggccaggatttcctgtgggggagacaacattggaagta
aaggtgtacattggtaccagcaaaagccaggccaggcccctgtggtggtc
gtctatgatgatcgcgaccggccctcagggatccctgagcgattctctgg
ctccaactctgggaacacggccaccctgaccatcagcagggtcgaagccg
gggatgaggccgactattactgtcaggtgtgggatagtggtagtgaccac
tgggttttcggcggagggaccaagctgaccgtcctaggatccggaaaggg
gcgcgccCATCATCATCATCATCAT (SEQ ID NO: 231)

TABLE 14B
Ab #E1-3H7 Constant Region amino acid sequences -
IgG1 LALA-aPD-L1 50-6B6.1 mut

CH1
Same as wild type (see Table 12B)
Hinge
Same as wild type (see Table 12B)
CH2 (identical to CH2 in Table 13B)
APE [symbol] GGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK (SEQ ID NO: 228)
CH3 (identical to CH3 in Table 14B)
GQPR [symbol] PQVYTLPPSRDELTKNQVSLTCLVKGFYPSDIAVEWESNGQPENN
YKTTPPVLDSDGSFFLYSKLTVDKSRWQQGNVFSCSVMH [symbol] ALHNHYTQKS
LSLSPGK (SEQ ID NO: 229)
C_{L} (CL in frame fusion with anti-PD-L1 scFv 50-
6B6.1 mut (lowercase letters), underlined
sequences denotes linkers (1) between C_{L} and scFv,
(2) between V_{H} and V_{L} within the scFv, (3)
between scFv and [symbol] (SEQ ID NO: 232))
GQPKAAPSVTLFPPSSEELQANKATLVCLISDFYPGAVTVAWKADGSPVK
AGVETTTPSKQSNNKYAASSYLSLTPEQWKSHRSYSCQVTHEGSTVEKTV
APTECSGGGGSGGGGSSMAqvqlvqsggevkkpgasvkvsckasgytlss
hgitwvrqapgqglewmgwisahnghasnaqkvedrvtmttdtstntaym
elrsltaddtavyycarvhaalyygmdvwgqgtlvtvssggggsggggag
gggssyeltqppsvslapgqsariscggdnigskgvhwyqqkpgqapvyv
vyddrdrpsgiperfsgsnsgntatltisrveagdeadyycqvwdsgsdh
wvfgggtkltvlGSGKGRA [symbol] (SEQ ID NO: 233)

TABLE 15A
Ab #E1-3H7 Constant Region nucleic acid
sequences - IgG1 LALA-aPD-L1 50-6B6.2

CH1
Same as wild type (see Table 12A)
Hinge
Same as wild type (see Table 12A)
CH2 (identical to CH2 in Table 13A)
GCACCTGAA [symbol] GGGGGACCGTCAGTCTTCCTCTTCCCCCCAAAACC
CAAGGACACCCTCATGATCTCCCGGACCCCTGAGGTCACATGCGTGGTGG
TGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGC
TGAATGGCAAGGAGTACAAGTGCAAGGTCTCCAACAAAGCCCTCCCAGCC
CCCATCGAGAAAACCATCTCCAAAGCCAAA (SEQ ID NO: 225)
CH3 (identical to CH3 in Table 14A)
GGGCAGCCCCGA [symbol] CCACAGGTGTACACCCTGCCCCCATCCCGGGATGA
GCTGACCAAGAACCAGGTCAGCCTGACCTGCCTGGTCAAAGGCTTCTATC
CCAGCGACATCGCCGTGGAGTGGGAGAGCAATGGGCAGCCGGAGAACAAC
TACAAGACCACGCCTCCCGTGCTGGACTCCGACGGCTCCTTCTTCCTCTA
CAGCAAGCTCACCGTGGACAAGAGCAGGTGGCAGCAGGGGAACGTCTTCT
CATGCTCCGTGATGCAT [symbol] GCTCTGCACAACCACTACACGCAGAAGAGC
CTCTCCCTGTCTCCGGGTAAATGA (SEQ ID NO: 226)
C_{L} (CL in frame fusion with anti-PD-L1 scFv 50-
6B6.2 (lowercase letters))
GGTCAGCCCAAGGCTGCCCCCTCGGTCACTCTGTTCCCGCCCTCCTCTGA
GGAGCTTCAAGCCAACAAGGCCACACTGGTGTGTCTCATAAGTGACTTCT
ACCCGGGAGCCGTGACAGTGGCCTGGAAGGCAGATGGCAGCCCCGTCAAG
GCGGGAGTGGAGACCACCACACCCTCCAAACAAAGCAACAACAAGTACGC
GGCCAGCAGCTATCTGAGCCTGACGCCTGAGCAGTGGAAGTCCCACAGAA
GCTACAGCTGCCAGGTCACGCATGAAGGGAGCACCGTGGAGAAGACAGTG
GCCCCTACAGAATGTTCAggtggcggcggttccggaggtggtggttcatc
gatggcccaggtgcagctggtgcagtctggaggtgaggtgaagaagccgg
gggcctcagtgaaggtctcctgcaaggcttctggttacaccttgagcagt
catggtataacctgggtgcgacaggcccctggacaagggcttgagtggat
gggatggatcagcgctcacaatggtcacgctagcaatgcacagaaggtgg
aggacagagtcactatgactactgacacatccacgaacacagcctacatg
gaactgaggagcctgacagctgacgacacggccgtgtattactgtgcgag
agtacatgctgccctctactatggtatggacgtctggggccaaggaaccc
tggtcaccgtctcctcaggtggcggcggttccggaggtggtggttctggc
ggtggtggcagcctgcctgtgctgactcagccaccctcagtgtccgcggc
cccgggacagacggccaggatttcctgtgggggaagcaacattggagata
aaggtgtccactggtaccagcagaagccaggccaggcccctgtgctggtc
atctatgatgatagcgaccggccctcagggatccctgagcgattctctgg
ctccaactctgggaacacggccaccctgaccatcagcagggtcgaagccg
gggatgaggccgactattactgtcaggtgtgggatagtagtagtgatcat
tgggtgttcggcggagggaccaagctgaccgtcctaggatccggaaaggg
gcgcgccCATCATCATCATCATCAT (SEQ ID NO: 234)

TABLE 15B
Ab #E1-3H7 Constant Region amino acid sequences -
IgG1 LALA-aPD-L1 50-6B6.2

CH1
Same as wild type (see Table 12B)
Hinge
Same as wild type (see Table 12B)
CH2 (identical to CH2 in Table 13B)
APE [symbol] GGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK (SEQ ID NO: 228)
CH3 (identical to CH3 in Table 14B)
GQPR [symbol] PQVYTLPPSRDELTKNQVSLTCLVKGFYPSDIAVEWESNGQPENN
YKTTPPVLDSDGSFFLYSKLTVDKSRWQQGNVFSCSVMH [symbol] ALHNHYTQKS
LSLSPGK (SEQ ID NO: 229)
C_{L} (CL in frame fusion with anti-PD-L1 scFv 50-
6B6.2 (lowercase letters), underlined sequences
denotes linkers (1) between C_{L} and scFv, (2)
between V_{H} and V_{L} within the scFv, (3) between
scFv and [symbol] (SEQ ID NO: 232))
GQPKAAPSVTLFPPSSEELQANKATLVCLISDFYPGAVTVAWKADGSPVK
AGVETTTPSKQSNNKYAASSYLSLTPEQWKSHRSYSCQVTHEGSTVEKTV
APTECSGGGGSGGGGSSMAqvqlvqsggevkkpgasvkvsckasgytlss
hgitwvrqapgqglewmgwisahnghasnaqkvedrvtmttdtstntaym
elrsltaddtavyycarvhaalyygmdvwgqgtlvtvssggggsggggsg
gggslpvltqppsvsaapgqtariscggsnigdkgvhwyqqkpgqapvlv
iyddsdrpsgiperfsgsnsgntatltisrveagdeadyycqvwdsssdh
wvfgggtkltvlGSGKGRA [symbol] (SEQ ID NO: 235)

TABLE 16A
Ab #E1-3H7 Constant Region nucleic acid
sequences - IgG1 LALA-aPD-L1 50-7B3

CH1
Same as wild type (see Table 12A)
Hinge
Same as wild type (see Table 12A)
CH2 (identical to CH2 in Table 13A)
GCACCTGAA [symbol] GGGGGACCGTCAGTCTTCCTCTTCCCCCCAAAACC
CAAGGACACCCTCATGATCTCCCGGACCCCTGAGGTCACATGCGTGGTGG
TGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGC
TGAATGGCAAGGAGTACAAGTGCAAGGTCTCCAACAAAGCCCTCCCAGCC
CCCATCGAGAAAACCATCTCCAAAGCCAAA (SEQ ID NO: 225)
CH3 (identical to CH3 in Table 14A)
GGGCAGCCCCGA [symbol] CCACAGGTGTACACCCTGCCCCCATCCCGGGATGA
GCTGACCAAGAACCAGGTCAGCCTGACCTGCCTGGTCAAAGGCTTCTATC
CCAGCGACATCGCCGTGGAGTGGGAGAGCAATGGGCAGCCGGAGAACAAC
TACAAGACCACGCCTCCCGTGCTGGACTCCGACGGCTCCTTCTTCCTCTA
CAGCAAGCTCACCGTGGACAAGAGCAGGTGGCAGCAGGGGAACGTCTTCT
CATGCTCCGTGATGCAT [symbol] GCTCTGCACAACCACTACACGCAGAAGAGC
CTCTCCCTGTCTCCGGGTAAATGA (SEQ ID NO: 226)
C_{L} (CL in frame fusion with anti-PD-L1 scFv 50-7B3
(lowercase letters))
GGTCAGCCCAAGGCTGCCCCCTCGGTCACTCTGTTCCCGCCCTCCTCTGA
GGAGCTTCAAGCCAACAAGGCCACACTGGTGTGTCTCATAAGTGACTTCT
ACCCGGGAGCCGTGACAGTGGCCTGGAAGGCAGATGGCAGCCCCGTCAAG
GCGGGAGTGGAGACCACCACACCCTCCAAACAAAGCAACAACAAGTACGC
GGCCAGCAGCTATCTGAGCCTGACGCCTGAGCAGTGGAAGTCCCACAGAA
GCTACAGCTGCCAGGTCACGCATGAAGGGAGCACCGTGGAGAAGACAGTG
GCCCCTACAGAATGTTCAGGTGGCGGCGGTTCCGGAGGTGGTGGTTCATC
GATGGCCCAGGTGCAGCTGGTGCAGTctggaggtgaggtgaagaagccgg
gggcctcagtgaaggtctcctgcaaggcttctggttacaccttgagcagt
catggtataacctgggtgcgacaggcccctggacaagggcttgagtggat
gggatggatcagcgctcacaatggtcacgctagcaatgcacagaaggtgg
aggacagagtcactatgactactgacacatccacgaacacagcctacatg
gaactgaggagcctgacagctgacgacacggccgtgtattactgtgcgag
agtacatgctgccctctactatggtatggacgtctggggccaaggaaccc
tggtcaccgtctcctcaggtggcggcggttccggaggtggtggttctggc
ggtggtggcagctcctatgagctgactcagccaccctcggtgtcagtggc
cccaggacagacggccaggattacctgtgggggaaacaacattggcaata
aaggtgtacactggtaccagcagaagccaggccaggcccctgtgctggtc
gtctatgatgatagcgaccggccctcagggatccctgagcgattctctgg
ctccaactctgggaacacggccaccctgaccatcagcagggtcgaagccg
gggatgaggccgactattactgtcaggtgtgggatagtagtagtgatcat
tgggtgttcggcggagggaccaagctgaccgtcctaggatccggaaaggg
gcgcgccCATCATCATCATCAT (SEQ ID NO: 236)

TABLE 16B
Ab #E1-3H7 Constant Region amino acid sequences -
IgG1 LALA-aPD-L1 50-7B3

CH1
Same as wild type (see Table 12B)
Hinge
Same as wild type (see Table 12B)
CH2 (identical to CH2 in Table 13B)
APE [symbol] GGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK (SEQ ID NO: 228)
CH3 (identical to CH3 in Table 14B)
GQPR [symbol] PQVYTLPPSRDELTKNQVSLTCLVKGFYPSDIAVEWESNGQPENN
YKTTPPVLDSDGSFFLYSKLTVDKSRWQQGNVFSCSVMH [symbol] ALHNHYTQKS
LSLSPGK (SEQ ID NO: 229)
C_{L} (CL in frame fusion with anti-PD-L1 scFv 50-7B3
(lowercase letters), underlined sequences denotes
linkers (1) between C_{L} and scFv, (2) between V_{H}
and V_{L} within the scFv, (3) between scFv and [symbol]
(SEQ ID NO: 232))
GQPKAAPSVTLFPPSSEELQANKATLVCLISDFYPGAVTVAWKADGSPVK
AGVETTTPSKQSNNKYAASSYLSLTPEQWKSHRSYSCQVTHEGSTVEKTV
APTECSGGGGSGGGGSSMAQVQLVQSGGEVKKPGASVKVSCKASGYTLSS
HGITWVRQAPGQGlewmgwisahnghasnaqkvedrvtmttdtstntaym
elrsltaddtavyycarvhaalyygmdvwgqgtlvtvssggggsggggsg
gggssyeltqppsvsvapgqtaritcggnnignkgvhwyqqkpgqapvlv
vyddsdrpsgiperfsgsnsgntatltisrveagdeadyycqvwdsssdh
wvfgggtkltvlgsgkgra [symbol] (SEQ ID NO: 237)

TABLE 17A
Ab #E1-3H7 Constant Region nucleic acid
sequences - IgG1 LALA-aPD-L1 50-5B9

CH1
Same as wild type (see Table 12A)
Hinge
Same as wild type (see Table 12A)
CH2 (identical to CH2 in Table 13A)
GCACCTGAA [symbol] GGGGGACCGTCAGTCTTCCTCTTCCCCCCAAAACC
CAAGGACACCCTCATGATCTCCCGGACCCCTGAGGTCACATGCGTGGTGG
TGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGC
TGAATGGCAAGGAGTACAAGTGCAAGGTCTCCAACAAAGCCCTCCCAGCC
CCCATCGAGAAAACCATCTCCAAAGCCAAA (SEQ ID NO: 225)
CH3 (identical to CH3 in Table 14A)
GGGCAGCCCCGA [symbol] CCACAGGTGTACACCCTGCCCCCATCCCGGGATGA
GCTGACCAAGAACCAGGTCAGCCTGACCTGCCTGGTCAAAGGCTTCTATC
CCAGCGACATCGCCGTGGAGTGGGAGAGCAATGGGCAGCCGGAGAACAAC
TACAAGACCACGCCTCCCGTGCTGGACTCCGACGGCTCCTTCTTCCTCTA
CAGCAAGCTCACCGTGGACAAGAGCAGGTGGCAGCAGGGGAACGTCTTCT
CATGCTCCGTGATGCATGG4GCTCTGCACAACCACTACACGCAGAAGAGC
CTCTCCCTGTCTCCGGGTAAATGA (SEQ ID NO: 226)
C_{L} (CL in frame fusion with anti-PD-Ll scFv 50-5B9
(lowercase letters))
GGTCAGCCCAAGGCTGCCCCCTCGGTCACTCTGTTCCCGCCCTCCTCTGA
GGAGCTTCAAGCCAACAAGGCCACACTGGTGTGTCTCATAAGTGACTTCT
ACCCGGGAGCCGTGACAGTGGCCTGGAAGGCAGATGGCAGCCCCGTCAAG
GCGGGAGTGGAGACCACCACACCCTCCAAACAAAGCAACAACAAGTACGC
GGCCAGCAGCTATCTGAGCCTGACGCCTGAGCAGTGGAAGTCCCACAGAA
GCTACAGCTGCCAGGTCACGCATGAAGGGAGCACCGTGGAGAAGACAGTG
GCCCCTACAGAATGTTCAggtggcggcggttccggaggtggtggttcatc
gatggcccaggtgcagctggtgcagtctggaggtgaggtgaagaagccgg
gggcctcagtgaaggtctcctgcaaggcttctggttacaccttgagcagt
catggtataacctgggtgcgacaggcccctggacaagggcttgagtggat
gggatggatcagcgctcacaatggtcacgctagcaatgcacagaaggtgg
aggacagagtcactatgactactgacacatccacgaacacagcctacatg
gaactgaggagcctgacagctgacgacacggccgtgtattactgtgcgag
agtacatgctgccctctactatggtatggacgtctggggccaaggaaccc
tggtcaccgtctcctcaggtggcggcggttccggaggtggtggttctggc
ggtggtggcagcctgcctgtgctgactcagccaccctcggtgtcagtggc
cctaggacagacggccaggattacctgtaggggaaacaacattggtggta
aaggtgtgcactggtaccagcagaagccaggccaggcccctgtgctggtc
gtctatgatgattactcccggcgctcaggaatccctgagcgattctctgg
ctcccactctgggagcgcggccaccctgaccatcagcagggtcgaggccg
gggatgaggccgactattactgtcaggtgtgggatagtagtagtgatcat
tgggtgttcggcggagggaccaagctgaccgtcctaggatccggaaaggg
gcgcgccCATCATCATCATCATCAT (SEQ ID NO: 238)

TABLE 17B
Ab #E1-3H7 Constant Region amino acid sequences -
IgG1 LALA-aPD-L1 50-5B9

CH1
Same as wild type (see Table 12B)
Hinge
Same as wild type (see Table 12B)
CH2 (identical to CH2 in Table 13B)
APE [symbol] GGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK (SEQ ID NO: 228)
CH3 (identical to CH3 in Table 14B)
GQPR [symbol] PQVYTLPPSRDELTKNQVSLTCLVKGFYPSDIAVEWESNGQPENN
YKTTPPVLDSDGSFFLYSKLTVDKSRWQQGNVFSCSVMH [symbol] ALHNHYTQKS
LSLSPGK (SEQ ID NO: 229)
C_{L} (CL in frame fusion with anti-PD-L1 scFv 50-5B9
(lower case letters), underlined sequences denotes
linkers (1) between C_{L} and scFv, (2) between V_{H}
and V_{L} within the scFv, (3) between scFv and [symbol]
(SEQ ID NO: 232))
GQPKAAPSVTLFPPSSEELQANKATLVCLISDFYPGAVTVAWKADGSPVK
AGVETTTPSKQSNNKYAASSYLSLTPEOWKSHRSYSCOVTHEGSTVEKTV
APTECSGGGGSGGGGSSmaavqlvqsggevkkpgasvkvsckasgytlss
hgitwvrqapgqglewmgwisahnghasnaqkvedrvtmttdtstntaym
elrsltaddtavyycarvhaalyygmdvwgqgtlvtvssggggsggggsg
gggslpvltqppsvsvalgqtaritcrgnniggkgvhwyqqkpgqapvlv
vyddysrrsgiperfsgshsgsaatltisrveagdeadyycqvwdsssdh
wvfgggtkltvlGSGKGRA [symbol] (SEQ ID NO: 239)

Multispecific antibodies (e.g., bispecific antibodies and trispecific antibodies) of the present invention can be constructed using methods known art. In some embodiments, the bi-specific antibody is a single polypeptide wherein the two scFv fragments are joined by a long linker polypeptide, of sufficient length to allow intramolecular association between the two scFv units to form an antibody. In other embodiments, the bi-specific antibody is more than one polypeptide linked by covalent or non-covalent bonds. In some embodiments, the amino acid linker (GGGGSGGGGS; “(G4S)2” (SEQ ID NO: 240)) that can be used with scFv fusion constructs described herein can be generated with a longer G4S linker (SEQ ID NO: 262) to improve flexibility. For example, the linker can also be “(G45)3” (e.g., GGGGSGGGGSGGGGS) (SEQ ID NO: 241); “(G45)4” (e.g., GGGGSGGGGSGGGGSGGGGS) (SEQ ID NO: 242); “(G4S)5” (e.g., GGGGSGGGGSGGGGSGGGGSGGGGS) (SEQ ID NO: 243); “(G45)6” (e.g., GGGGSGGGGSGGGGSGGGGSGGGGSGGGGS) (SEQ ID NO: 244); “(G45)7” (e.g., GGGGSGGGGSGGGGSGGGGSGGGGSGGGGSGGGGS) (SEQ ID NO: 245); and the like. For example, use of the (G4S)5 linker (SEQ ID NO: 243) can provide more flexibility and can improve expression. In some embodiments, the linker can also be (GS)ₙ (SEQ ID NO: 246), (GGS)ₙ (SEQ ID NO: 247), (GGGS)ₙ (SEQ ID NO: 248), (GGSG)ₙ (SEQ ID NO: 249), (GGSGG)ₙ (SEQ ID NO: 250), or (GGGGS)ₙ (SEQ ID NO: 251), wherein n is 1, 2, 3, 4, 5, 6, 7, 8, 9, or 10. Non-limiting examples of linkers known to those skilled in the art that can be used are described in U.S. Pat. No. 9,708,412; U.S. Patent Application Publication Nos. US 20180134789 and US 20200148771; and PCT Publication No. WO2019051122 (each of which are incorporated by reference in their entireties).

In another embodiment, the multispecific antibodies (e.g., bispecific antibodies and trispecific antibodies) can be constructed using the “knob into hole” method (Ridgway et al, Protein Eng 7:617-621 (1996)). In this method, the Ig heavy chains of the two different variable domains are reduced to selectively break the heavy chain pairing while retaining the heavy-light chain pairing. The two heavy-light chain heterodimers that recognize two different antigens are mixed to promote heteroligation pairing, which is mediated through the engineered “knob into holes” of the CH3 domains.

In another embodiment, multispecific antibodies (e.g., bispecific antibodies and trispecific antibodies) can be constructed through exchange of heavy-light chain dimers from two or more different antibodies to generate a hybrid antibody where the first heavy-light chain dimer recognizes PD-L1 and the second heavy-light chain dimer recognizes a second antigen. In some embodiments, the bi-specific antibody can be constructed through exchange of heavy-light chain dimers from two or more different antibodies to generate a hybrid antibody where the first heavy-light chain dimer recognizes a second antigen and the second heavy-light chain dimer recognizes PD-L1. The mechanism for heavy-light chain dimer is similar to the formation of human IgG₄, which also functions as a bispecific molecule. Dimerization of IgG heavy chains is driven by intramolecular force, such as the pairing the CH3 domain of each heavy chain and disulfide bridges. Presence of a specific amino acid in the CH3 domain (R409) has been shown to promote dimer exchange and construction of the IgG₄ molecules. Heavy chain pairing is also stabilized further by interheavy chain disulfide bridges in the hinge region of the antibody. Specifically, in IgG₄, the hinge region contains the amino acid sequence Cys-Pro-Ser-Cys (SEQ ID NO: 260) (in comparison to the stable IgG1 hinge region which contains the sequence Cys-Pro-Pro-Cys (SEQ ID NO: 261)) at amino acids 226- 230. This sequence difference of Serine at position 229 has been linked to the tendency of IgG₄ to form intrachain disulfides in the hinge region (Van der Neut Kolfschoten, M. et al, 2007, Science 317: 1554-1557 and Labrijn, A. F. et al, 2011, Journal of Immunol 187:3238-3246).

Therefore, bi-specific antibodies of the present invention can be created through introduction of the R409 residue in the CH3 domain and the Cys-Pro-Ser-Cys (SEQ ID NO: 260) sequence in the hinge region of antibodies that recognize PD-L1 or a second antigen, so that the heavy-light chain dimers exchange to produce an antibody molecule with one heavy-light chain dimer recognizing PD-L1 and the second heavy-light chain dimer recognizing a second antigen, wherein the second antigen is any antigen disclosed herein. Known IgG₄ molecules can also be altered such that the heavy and light chains recognize PD-L1 or a second antigen, as disclosed herein. Use of this method for constructing the bi-specific antibodies of the present invention can be beneficial due to the intrinsic characteristic of IgG₄ molecules wherein the Fc region differs from other IgG subtypes in that it interacts poorly with effector systems of the immune response, such as complement and Fc receptors expressed by certain white blood cells. This specific property makes these IgG4-based bi-specific antibodies attractive for therapeutic applications, in which the antibody is required to bind the target(s) and functionally alter the signaling pathways associated with the target(s), however not trigger effector activities.

In some embodiments, mutations are introduced to the constant regions of the bsAb such that the antibody dependent cell-mediated cytotoxicity (ADCC) activity of the bsAb is altered. For example, the mutation is a LALA mutation in the CH2 domain. In one aspect, the bsAb contains mutations on one scFv unit of the heterodimeric bsAb, which reduces the ADCC activity. In another aspect, the bsAb contains mutations on both chains of the heterodimeric bsAb, which completely ablates the ADCC activity. For example, the mutations introduced one or both scFv units of the bsAb are LALA mutations in the CH2 domain. These bsAbs with variable ADCC activity can be optimized such that the bsAbs exhibits maximal selective killing towards cells that express one antigen that is recognized by the bsAb, however exhibits minimal killing towards the second antigen that is recognized by the bsAb.

The bi-specific antibodies disclosed herein can be useful in treatment of chronic infections, diseases, or medical conditions, for example, cancer.

### Use of Antibodies Against PD-L1

Antibodies of the invention specifically binding a PD-L1 protein, or a fragment thereof, can be administered for the treatment a PD-L1 associated disease or disorder. A “PD-L1-associated disease or disorder” includes disease states and/or symptoms associated with a disease state, where increased levels of PD-L1 and/or activation of cellular signaling pathways involving PD-L1 are found. Exemplary PD-L1-associated diseases or disorders include, but are not limited to, cancer and auto-immune diseases.

Antibodies of the invention, including bi-specific, polyclonal, monoclonal, humanized and fully human antibodies, can be used as therapeutic agents. Such agents will generally be employed to treat or prevent cancer in a subject, increase vaccine efficiency or augment a natural immune response. An antibody preparation, for example, one having high specificity and high affinity for its target antigen, is administered to the subject and will generally have an effect due to its binding with the target. Administration of the antibody can abrogate or inhibit or interfere with an activity of the PD-L1 protein.

Antibodies of the invention specifically binding a PD-L1 protein or fragment thereof can be administered for the treatment of a cancer in the form of pharmaceutical compositions. Principles and considerations involved in preparing therapeutic pharmaceutical compositions comprising the antibody, as well as guidance in the choice of components are provided, for example, in: Remington: The Science And Practice Of Pharmacy 20th ed. (Alfonso R. Gennaro, et al, editors) Mack Pub. Co., Easton, Pa., 2000; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

A therapeutically effective amount of an antibody of the invention can be the amount needed to achieve a therapeutic objective. As noted herein, this can be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. The dosage administered to a subject (e.g., a patient) of the antigen-binding polypeptides described herein is typically 0.1 mg/kg to 100 mg/kg of the patient's body weight, between 0.1 mg/kg and 20 mg/kg of the patient's body weight, or 1 mg/kg to 10 mg/kg of the patient's body weight. Human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation. Common ranges for therapeutically effective dosing of an antibody or antibody fragment of the invention can be, by way of nonlimiting example, from about 0.1 mg/kg body weight to about 50 mg/kg body weight. Common dosing frequencies can range, for example, from twice daily to once a week.

Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90: 7889-7893 (1993)). The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine (e.g. IL-15), chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g. , films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

An antibody according to the invention can be used as an agent for detecting the presence of PD-L1 (or a protein fragment thereof) in a sample. For example, the antibody can contain a detectable label. Antibodies can be polyclonal or monoclonal. An intact antibody, or a fragment thereof (e.g., F_{ab}, scFv, or F _{(ab)2}) can be used. The term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” can include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA includes Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations.

Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Antibodies directed against a PD-L1 protein (or a fragment thereof) can be used in methods known within the art relating to the localization and/or quantitation of a PD-L1 protein (e.g., for use in measuring levels of the PD-L1 protein within appropriate physiological samples, for use in diagnostic methods, for use in imaging the protein, and the like). In a given embodiment, antibodies specific to a PD-L1 protein, or derivative, fragment, analog or homolog thereof, that contain the antibody derived antigen binding domain, are utilized as pharmacologically active compounds (referred to herein as “therapeutics”).

An antibody of the invention specific for a PD-L1 protein can be used to isolate a PD-L1 polypeptide by standard techniques, such as immunoaffinity, chromatography or immunoprecipitation. Antibodies directed against a PD-L1 protein (or a fragment thereof) can be used diagnostically to monitor protein levels in tissue as part of a clinical testing procedure, e.g., to, for example, determine the efficacy of a given treatment regimen.

Detection can be facilitated by coupling (i.e., physically linking) the antibody to a detectable substance. Examples of detectable substances include, but are not limited to, various enzymes, prosthetic groups, fluorescent materials, luminescent materials, bioluminescent materials, and radioactive materials. Non-limiting examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³²P or ³H.

The antibodies or agents of the invention (also referred to herein as “active compounds”), and derivatives, fragments, analogs and homologs thereof, can be incorporated into pharmaceutical compositions suitable for administration. Such pharmaceutical compositions can comprise the antibody or agent and a pharmaceutically acceptable carrier. As used herein, the term “pharmaceutically acceptable carrier” can include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Non-limiting examples of such carriers or diluents include water, saline, ringer's solutions, dextrose solution, and 5% human serum albumin. Liposomes and non-aqueous vehicles such as fixed oils can also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In embodiments, the composition is sterile and is fluid to the extent that easy syringeability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. For example, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### Methods of Treatment

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can refer to prolonging survival as compared to expected survival if not receiving treatment. Those in need of treatment include those already with the condition or disorder as well as those prone to have the condition or disorder or those in which the condition or disorder is to be prevented.

The invention provides for both prophylactic and therapeutic methods of treating a subject at risk of (or susceptible to) a cancer, or other cell proliferation-related diseases or disorders. Such diseases or disorders include but are not limited to, e.g., those diseases or disorders associated with aberrant expression of PD-L1. For example, the methods are used to treat, prevent or alleviate a symptom of cancer. In an embodiment, the methods are used to treat, prevent or alleviate a symptom of a solid tumor. Non-limiting examples of other tumors that can be treated by embodiments herein comprise lung cancer, ovarian cancer, prostate cancer, colon cancer, cervical cancer, brain cancer, skin cancer, liver cancer, pancreatic cancer or stomach cancer. Additionally, the methods of the invention can be used to treat hematologic cancers such as leukemia and lymphoma. Alternatively, the methods can be used to treat, prevent or alleviate a symptom of a cancer that has metastasized.

Accordingly, in one aspect, the invention provides methods for preventing, treating or alleviating a symptom cancer or a cell proliferative disease or disorder in a subject by administering to the subject a monoclonal antibody, scFv antibody or bi- specific antibody of the invention. For example, an anti-PD-L1 antibody can be administered in therapeutically effective amounts.

Subjects at risk for cancer or cell proliferation-related diseases or disorders can include patients who have a family history of cancer or a subject exposed to a known or suspected cancer-causing agent. Administration of a prophylactic agent can occur prior to the manifestation of cancer such that the disease is prevented or, alternatively, delayed in its progression.

In another aspect, tumor cell growth is inhibited by contacting a cell with an anti-PD-L1 antibody of the invention. The cell can be any cell that expresses PD-L1.

The invention further provides for both prophylactic and therapeutic methods of treating a subject at risk of (or susceptible to) a chronic viral, bacterial or parasitic infection. The invention also provides for therapeutic methods for both prophylactic and therapeutic methods of treating a subject at risk of a disease or disorder or condition associated with T-cell exhaustion or a risk of developing T-cell exhaustion. The invention also provides for therapeutic methods for both prophylactic and therapeutic methods of treating a subject at risk of a disease or disorder or condition associated with T-cell exhaustion or a risk of developing T-cell exhaustion. Such diseases or disorder include, but are not limited to HIV, AIDS, and chronic bacterial, viral or parasitic infections. Other such chronic infections include those caused by, for example, hepatitis B virus (HBV), hepatitis C virus (HCV), herpes simplex virus 1 (HSV-1), H pylori, or Toxoplasma gondii.

Also included in the invention are methods of increasing or enhancing an immune response to an antigen. An immune response is increased or enhanced by administering to the subject a monoclonal antibody, scFv antibody, or bi-specific antibody of the invention. The immune response is augmented for example by augmenting antigen specific T effector function. The antigen is a viral (e.g. HIV), bacterial, parasitic or tumor antigen. The immune response is a natural immune response. By natural immune response is meant an immune response that is a result of an infection. The infection is a chronic infection. Increasing or enhancing an immune response to an antigen can be measured by a number of methods known in the art. For example, an immune response can be measured by measuring any one of the following: T cell activity, T cell proliferation, T cell activation, production of effector cytokines, and T cell transcriptional profile. Alternatively, the immune response is a response induced due to a vaccination.

Accordingly, in another aspect the invention provides a method of increasing vaccine efficiency by administering to the subject a monoclonal antibody or scFv antibody of the invention and a vaccine. The antibody and the vaccine are administered sequentially or concurrently. The vaccine is a tumor vaccine a bacterial vaccine or a viral vaccine.

### Combinatory Methods

Compositions of the invention as described herein can be administered in combination with a chemotherapeutic agent. Chemotherapeutic agents that can be administered with the compositions described herein include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In additional embodiments, the compositions of the invention as described herein can be administered in combination with cytokines. Cytokines that may be administered with the compositions include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions described herein can be administered in combination with other therapeutic or prophylactic regimens, such as, for example, radiation therapy.

In some embodiments, the compositions described herein can be administered in combination with other immunotherapeutic agents. Non-limiting examples of immunotherapeutic agents include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8.

The invention provides for methods of treating cancer in a patient by administering two antibodies that bind to the same epitope of the PD-L1 protein or, alternatively, two different epitopes of the PD-1 protein. Alternatively, the cancer can be treated by administering a first antibody that binds to PD-L1 and a second antibody that binds to a protein other than PD-L1. In other embodiments, the cancer can be treated by administering a bispecific antibody that binds to PD-L1 and that binds to a protein other than PD-L1. For example, the other protein other than PD-L1 can include, but is not limited to, GITR. For example, the other protein other than PD-L1 is a tumor-associated antigen; the other protein other than PD-L1 can also be a cytokine.

In some embodiments, the invention provides for the administration of an anti-PD-L1 antibody alone or in combination with an additional antibody that recognizes another protein other than PD-L1, with cells that are capable of effecting or augmenting an immune response. For example, these cells can be peripheral blood mononuclear cells (PBMC), or any cell type that is found in PBMC, e.g., cytotoxic T cells, macrophages, and natural killer (NK) cells.

Additionally, the invention provides administration of an antibody that binds to the PD-L1 protein and an anti-neoplastic agent, such a small molecule, a growth factor, a cytokine or other therapeutics including biomolecules such as peptides, peptidomimetics, peptoids, polynucleotides, lipid-derived mediators, small biogenic amines, hormones, neuropeptides, and proteases. Small molecules include, but are not limited to, inorganic molecules and small organic molecules. Suitable growth factors or cytokines include an IL-2, GM-CSF, IL-12, and TNF-alpha. Small molecule libraries are known in the art. (See, Lam, Anticancer Drug Des., 12: 145, 1997.)

### Chimeric Antigen Receptor (CAR) T-Cell Therapies

Cellular therapies, such as chimeric antigen receptor (CAR) T-cell therapies, are also provided herein. CAR T-cell therapies redirect a patient's T-cells to kill tumor cells by the exogenous expression of a CAR. A CAR can be a membrane spanning fusion protein that links the antigen recognition domain of an antibody to the intracellular signaling domains of the T-cell receptor and co-receptor. A suitable cell can be used, that is put in contact with an anti-PD-L1 antibody of the present invention (or alternatively engineered to express an anti-PD-L1 antibody as described herein). Solid tumors offer unique challenges for CAR-T therapies. Unlike blood cancers, tumor-associated target proteins are overexpressed between the tumor and healthy tissue resulting in on-target/off-tumor T-cell killing of healthy tissues. Furthermore, immune repression in the tumor microenvironment (TME) limits the activation of CAR-T cells towards killing the tumor. Upon such contact or engineering, the cell can then be introduced to a cancer patient in need of a treatment. The cancer patient may have a cancer of any of the types as disclosed herein. The cell (e.g., a T cell) can be, for instance, a tumor-infiltrating T lymphocyte, a CD4+ T cell, a CD8+ T cell, or the combination thereof, without limitation. Exemplary CARS useful in aspects of the invention include those disclosed in, for example, PCT/US2015/067225 and PCT/US2019/022272, each of which are hereby incorporated by reference in their entireties.

In one embodiment, the PD-L1 antibodies discussed herein can be used in the construction of multi-specific antibodies or as the payload for a CAR-T cell. For example, in one embodiment, the anti-PD-L1 antibodies discussed herein can be used for the targeting of the CARS (i.e., as the targeting moiety). In another embodiment, the anti-PD-L1 antibodies discussed herein can be used as the targeting moiety, and a different PD-L1 antibody that targets a different epitope can be used as the payload. In another embodiment, the payload can be an immunomodulatory antibody payload. In some embodiments, the PD-Llantibodies described herein can be used as targeting moieties in CARs (e.g., kill PDL-1⁺ tumor cells) or as a secreted checkpoint blockade antibody to reverse T cell exhaustion.

For example, embodiments of the invention comprise chimeric antigen receptor (CAR) comprising an intracellular signaling domain, a transmembrane domain and an extracellular domain. In embodiments, the extracellular domain is an isolated monoclonal antibody or antigen-binding fragment thereof that binds to human Programmed death-ligand 1 (PD-L1) protein. For example, the monoclonal antibody or fragment thereof comprises a heavy chain, light chain, or combination thereof, wherein the heavy chain comprises a CDR1 comprising G-(X₁)-T-(X₂)-SS-(X₃X₄) (SEQ ID NO: 47), G-(X₁)-T-(X₂)-(X₁₃X₁₄)-(X₃X₄) (SEQ ID NO: 205), G-(X1)-TF-(X₁₃X₁₄)-Y-(X₄) (SEQ ID NO: 206), a CDR2 comprising I-(X₈X₉X₁₀ X₁₁-G-(X₁₂)-A (SEQ ID NO: 51), or II-(X₁₅)-IFG-(X₁₆)-A (SEQ ID NO: 207), and/or a CDR3 comprising ARGRQMFGAGIDF (SEQ ID NO: 6) or ARVHAALYYGMDV (SEQ ID NO: 14), TTGGLGLVYPYYNYIDV (SEQ ID NO: 99), AKVHPVFSYALDV (SEQ ID NO: 100), AEEGAFNSLAI (SEQ ID NO: 101), ARDGSGYDSAGMDD (SEQ ID NO: 102), ARGFGGPDY (SEQ ID NO: 103), ARVHGALYYGMDV (SEQ ID NO: 104), ASGSIVGAAYAFDI (SEQ ID NO: 105), ARDRSEGGFDP (SEQ ID NO: 106), or AEEGAFNSLAI (SEQ ID NO: 107); and wherein the light chain comprises a CDR1 comprising S-(X₁₇X₁₈)I-(X₁₉)-SNY (SEQ ID NO: 208) or NIG-(X₅)-K-(X₂₀) (SEQ ID NO: 48), a CDR2 comprising (X₂₁)-DN (SEQ ID NO: 209), (X₂₂)-NN (SEQ ID NO: 210), or DD-X₆ (SEQ ID NO: 49), and/or a CDR3 comprising QSYDSNNRHVI (SEQ ID NO: 22), QVWDS-(X₇)-SDHWV (SEQ ID NO: 50), QVWDSSGDLWV (SEQ ID NO: 126), AAWDDSLNGLV (SEQ ID NO: 127), QSYDGITVI (SEQ ID NO: 128), QSYDSSNHWV (SEQ ID NO: 129), AVWDDSLSGVV (SEQ ID NO: 131), MIWHSSAYV (SEQ ID NO: 132), NSRDISDNQWQWI (SEQ ID NO: 134), or QSYDSSNHVV (SEQ ID NO: 135).

The CAR according to the invention can comprise at least one transmembrane polypeptide comprising at least one extracellular ligand-biding domain and; one transmembrane polypeptide comprising at least one intracellular signaling domain; such that the polypeptides assemble together to form a Chimeric Antigen Receptor.

The term “extracellular ligand-binding domain” as used herein is defined as an oligo- or polypeptide that is capable of binding a ligand. For example, the domain will be capable of interacting with a cell surface molecule. For example, the extracellular ligand-binding domain can be chosen to recognize a ligand that acts as a cell surface marker on target cells associated with a particular disease state.

In one embodiment, the extracellular ligand-binding domain can comprise an antigen binding domain derived from an antibody against an antigen of the target. For example, the target can be PD-L1. Thus, the CAR can be specific for PD-L1. In an embodiment, said extracellular ligand-binding domain is a single chain antibody fragment (scFv) comprising the light (VL) and the heavy (VH) variable fragment of a target antigen specific monoclonal antibody joined by a flexible linker. For example, said scFv antibody is specific for PD-L1. It is understood, however, that binding domains other than scFv can also be used for predefined targeting of lymphocytes, such as camelid single-domain antibody fragments or receptor ligands, antibody binding domains, antibody hypervariable loops or CDRs as non limiting examples.

In embodiments said transmembrane domain comprises a stalk region between said extracellular ligand-binding domain and said transmembrane domain. The term “stalk region” can refer to any oligo- or polypeptide that functions to link the transmembrane domain to the extracellular ligand-binding domain. In particular, stalk region(s) is/are used to provide more flexibility and accessibility for the extracellular ligand-binding domain. A stalk region can comprise up to 300 amino acids, such as 10 to 100 amino acids. In embodiments, the stalk region comprises 25 to 50 amino acids. Stalk region can be derived from all or part of naturally occurring molecules, such as from all or part of the extracellular region of CD8, CD4 or CD28, or from all or part of an antibody constant region. Alternatively the stalk region can be a synthetic sequence that corresponds to a naturally occurring stalk sequence, or may be an entirely synthetic stalk sequence. In a preferred embodiment said stalk region is a part of human CD8 alpha chain.

In embodiments, the transmembrane domain can comprise CD28.

The signal transducing domain or intracellular signaling domain of the CAR of the invention is responsible for intracellular signaling following the binding of extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” can refer to the portion of a protein which transduces the effector signal function signal and directs the cell to perform a specialized function.

Signal transduction domain can comprise two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non limiting examples those derived from TCR zeta, FcR gamma, FcR beta, FcR epsilon, CD3 gamma, CD3 delta, CD3 epsilon, CD5, CD22, CD79a, CD79b and CD66d. In a preferred embodiment, the signaling transducing domain of the CAR can comprise the CD3 zeta signaling domain, or the intracytoplasmic domain of the Fc epsilon RI beta or gamma chains. In another preferred embodiment, the signaling is provided by CD3 zeta together with co-stimulation provided by CD28 and a tumor necrosis factor receptor (TNFr), such as 4-1BB or OX40), for example.

In embodiments, the intracellular signaling domain of the CAR of the present invention comprises a co-stimulatory signal molecule. In some embodiments the intracellular signaling domain contains 2, 3, 4 or more co-stimulatory molecules in tandem. A co-stimulatory molecule is a cell surface molecule other than an antigen receptor or their ligands that is required for an efficient immune response.

“Co-stimulatory ligand” can refer to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-IBB, OX40, CD30, CD40, PD-1, ICOS, lymphocyte function-associated antigen-1 (LFA-1), CD2, CD7, LIGHT, NKG2C, B7-H3, a ligand that specifically binds with CD83.

A “co-stimulatory molecule” can refer to the cognate binding partner on a T-cell that specifically binds with a co-stimulatory ligand, thereby mediating a co-stimulatory response by the cell, such as, but not limited to proliferation. Co-stimulatory molecules include, but are not limited to an MHC class 1 molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

In embodiments, the choice of CD28 as a co-stimulatory domain for the CARs can be based in the fact that CD28 CARs direct an active proliferative response and enhance effector functions, whereas 4-1BB-based CARs induce a more progressive T cell accumulation that may counterweigh for less immediate effectiveness. In one embodiment, the CD28 is replaced by 41BB in the CAR constructs.

In another embodiment, said signal transducing domain is a TNFR-associated Factor 2 (TRAF2) binding motifs, intracytoplasmic tail of costimulatory TNFR member family. Cytoplasmic tail of costimulatory TNFR family member contains TRAF2 binding motifs consisting of the major conserved motif (P/S/A)X(Q/E)E) or the minor motif (PXQXXD), wherein X is any amino acid. TRAF proteins are recruited to the intracellular tails of many TNFRs in response to receptor trimerization.

The distinguishing features of appropriate transmembrane polypeptides comprise the ability to be expressed at the surface of an immune cell, in particular lymphocyte cells or Natural killer (NK) cells, and to interact together for directing cellular response of immune cell against a predefined target cell. The different transmembrane polypeptides of the CAR of the present invention comprising an extracellular ligand-biding domain and/or a signal transducing domain interact together to take part in signal transduction following the binding with a target ligand and induce an immune response. The transmembrane domain can be derived either from a natural or from a synthetic source. The transmembrane domain can be derived from any membrane-bound or transmembrane protein.

The term “a part of” used herein can refer to any subset of the molecule, that is a shorter peptide. Alternatively, amino acid sequence functional variants of the polypeptide can be prepared by mutations in the DNA which encodes the polypeptide. Such variants or functional variants include, for example, deletions from, or insertions or substitutions of, residues within the amino acid sequence. Any combination of deletion, insertion, and substitution may also be made to arrive at the final construct, provided that the final construct possesses the desired activity, especially to exhibit a specific anti-target cellular immune activity. The functionality of the CAR of the invention within a host cell is detectable in an assay suitable for demonstrating the signaling potential of said CAR upon binding of a particular target. Such assays are available to the skilled person in the art. For example, this assay allows the detection of a signaling pathway, triggered upon binding of the target, such as an assay involving measurement of the increase of calcium ion release, intracellular tyrosine phosphorylation, inositol phosphate turnover, or interleukin (IL) 2, interferon γ, GM-CSF, IL-3, IL-4 production thus effected.

### Cells that Express a CAR

Embodiments of the invention include cells that express a CAR (i.e, CARTS). The cell may be of any kind, including an immune cell capable of expressing the CAR for cancer therapy or a cell, such as a bacterial cell, that harbors an expression vector that encodes the CAR. As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid. In embodiments of the invention, a host cell is a T cell, including a cytotoxic T cell (also known as TC, Cytotoxic T Lymphocyte, CTL, T-Killer cell, cytolytic T cell, CD8+ T-cells or killer T cell); NK cells and NKT cells are also encompassed in the invention.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

The cells can be autologous cells, syngeneic cells, allogenic cells and even in some cases, xenogeneic cells.

In many situations one may wish to be able to kill the modified CTLs, where one wishes to terminate the treatment, the cells become neoplastic, in research where the absence of the cells after their presence is of interest, or other event. For this purpose one can provide for the expression of certain gene products in which one can kill the modified cells under controlled conditions, such as inducible suicide genes.

### Armed CARTS

The invention further includes CARTS that are modified to secrete one or more polypeptides. Armed CARTS have the advantage of simultaneously secreting a polypeptide at the targeted site, e.g. tumor site. The polypeptide can be for example be an antibody or cytokine. For example, the antibody is specific for PD-L1, such as antibodies and fragments described herein. In other embodiments, the secreted antibody can be an antibody specific for CAIX, GITR, PD-L2, PD-1, or CCR4 (See, for example, sequences described in PCT Publication No. WO2016/100985, the application which is incorporated by reference in its entirety).

Armed CART can be constructed by including a nucleic acid encoding the secreted polypeptide of interest after the intracellular signaling domain. In embodiments, there is an internal ribosome entry site, (IRES), positioned between the intracellular signaling domain and the polypeptide of interest. One skilled in the art can appreciate that more than one polypeptide can be expressed by employing multiple IRES sequences in tandem.

In embodiments, CART cells can be maintained with the use of cytokines such as, for example, IL-2, IL-4, IL-7, IL-9, IL-15 and IL-21.

Cytokines sharing the yc receptor, like IL-2, IL-4, IL-7, IL-9, IL-15 and IL-21 are important for the development and maintenance of memory T cells. Among them, IL-21 promote a less differentiated phenotype, associated with an enrichment of tumor-specific CD8 T cells, with increased anti-tumor effect in a mouse melanoma model when compared to IL-2 or IL-15.

In certain embodiments, CART cells are maintained with IL-21.

### Introduction of Constructs into CTLs

Expression vectors that encode the CARs can be introduced as one or more DNA molecules or constructs, where there may be at least one marker that will allow for selection of host cells that contain the construct(s).

The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be introduced into the CTL by any convenient means. The constructs may be integrated and packaged into non-replicating, defective viral genomes like Adenovirus, Adeno-associated virus (AAV), or Herpes simplex virus (HSV) or others, including retroviral vectors or lentiviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In some instances, one may have a target site for homologous recombination, where it is desired that a construct be integrated at a particular locus. For example,) can knock-out an endogenous gene and replace it (at the same locus or elsewhere) with the gene encoded for by the construct using materials and methods as are known in the art for homologous recombination. For homologous recombination, one may use either .OMEGA. or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs may be introduced as a single DNA molecule encoding at least the CAR and optionally another gene, or different DNA molecules having one or more genes. Other genes include genes that encode therapeutic molecules or suicide genes, for example. The constructs may be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

### Methods of Use of Cells that Express a CAR

The cells described herein can be used for treating a cancer, or other cell proliferation-related diseases or disorders. Such diseases or disorders include but are not limited to, e.g., those diseases or disorders associated with aberrant expression of PD-L1. In another embodiment, said isolated cell according to the invention can be used in the manufacture of a medicament for treatment a cancer, or other cell proliferation-related diseases or disorders. Such diseases or disorders include but are not limited to, e.g., those diseases or disorders associated with aberrant expression of PD-L1.

Embodiments described herein rely on methods for treating patients in need thereof, said method comprising at least one of the following steps: (a) providing a chimeric antigen receptor cells according to the invention and (b) administrating the cells to said patient.

Said treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

The invention is particularly suited for allogenic immunotherapy, insofar as it enables the transformation of T-cells, typically obtained from donors, into non-alloreactive cells. This may be done under standard protocols and reproduced as many times as needed. The resulted modified T cells may be pooled and administrated to one or several patients, being made available as an “off the shelf” therapeutic product.

Cancers that may be treated using the antibody or CAR compositions described herein include tumors that are not vascularized, or not yet substantially vascularized, as well as vascularized tumors. The cancers may comprise nonsolid tumors (such as hematological tumors, for example, leukemias and lymphomas) or may comprise solid tumors. Types of cancers to be treated with the antibodies and CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. For example, cancers of which a checkpoint blockade is a standard therapy for multiple malignancies (referred to herein as “Checkpoint Blockade Cancers”) can be treated with the antibody and/or CAR compositions described herein. Checkpoint Blockade Cancers include, but are not limited to, melanoma, non-small-cell lung cancer (NSCLC), small cell lung cancer (SCLC), renal cell carcinoma (RCC), chronic lymphocytic leukemia (CLL; such as B cell CLL or T cell CLL), classical Hodgkin lymphoma (cHL), head and neck squamous cell carcinoma (HNSCC), colorectal cancer (CRC), gastric cancer, hepatocellular carcinoma (HCC), primary mediastinal large B-cell lymphoma (PMLBCL), bladder cancer, urothelial cancer, endometrial cancer, cervical cancer, breast cancer (e.g., triple negative breast cancer), Merkel cell carcinoma (MCC), and microsatellite instability high (MSI-H) or DNA mismatch repair deficient (dMMR) adult and pediatric solid tumors (doi: 10.1016/j.csbj.2019.03.006). The treatments described herein can also include other cancers that are under investigation for checkpoint blockade therapies. Without wishing to be bound by theory, RCC and B-CLL mouse models can be used for treatment with CAR T factories, which are models correlated to the human disease.

For example, treatment can be antibody and/or CAR-T treatment in combination with one or more therapies against cancer selected from the group of antibodies therapy, chemotherapy, cytokines therapy, dendritic cell therapy, gene therapy, hormone therapy, laser light therapy and radiation therapy.

According to an embodiment of the invention, said treatment can be administrated into patients undergoing an immunosuppressive treatment. Indeed, the present invention can rely on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the patient.

In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAM PATH. In another embodiment, the cell compositions of the present invention are administered following B-cell ablative therapy such as agents that react with CD20, e.g., Rittman. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery. Said modified cells obtained by any one of the methods described here can be used in a particular aspect of the invention for treating patients in need thereof against Host versus Graft (HvG) rejection and Graft versus Host Disease (GvHD); therefore in the scope of the present invention is a method of treating patients in need thereof against Host versus Graft (HvG) rejection and Graft versus Host Disease (GvHD) comprising treating said patient by administering to said patient an effective amount of modified cells comprising inactivated TCR alpha and/or TCR beta genes.

### Administration of Cells

The invention is suited for allogenic immunotherapy, insofar as it enables the transformation of T-cells, typically obtained from donors, into non-alloreactive cells. This may be done under standard protocols and reproduced as many times as needed. The resulted modified T cells may be pooled and administrated to one or several patients, being made available as an “off the shelf” therapeutic product.

Depending upon the nature of the cells, the cells may be introduced into a host organism, e.g. a mammal, in a wide variety of ways. The cells may be introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, generally being injected at or near the site of interest. The cells may be in a physiologically-acceptable medium.

In some embodiments, the cells are encapsulated to inhibit immune recognition and placed at the site of the tumor.

The cells can be administered as desired. Depending upon the response desired, the manner of administration, the life of the cells, the number of cells present, various protocols may be employed. The number of administrations will depend upon the factors described above at least in part.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermaly, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, such as 10⁵ to 10⁶ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

It should be appreciated that the system is subject to many variables, such as the cellular response to the ligand, the efficiency of expression and, as appropriate, the level of secretion, the activity of the expression product, the particular need of the patient, which may vary with time and circumstances, the rate of loss of the cellular activity as a result of loss of cells or expression activity of individual cells, and the like. Therefore, it is expected that for each individual patient, even if there were universal cells which could be administered to the population at large, each patient would be monitored for the proper dosage for the individual, and such practices of monitoring a patient are routine in the art.

### Nucleic Acid-Based Expression Systems

The CARs of the present invention may be expressed from an expression vector. Recombinant techniques to generate such expression vectors are well known in the art.

The term “vector” can refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” can refer to any type of genetic construct comprising a nucleic acid coding for an RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

A “promoter” can refer to a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter can comprise a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter can be one naturally associated with a nucleic acid sequence, as may be obtained by isolating the 5 prime' non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR.TM., in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages, and these may be used in the invention.

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Splicing sites, termination signals, origins of replication, and selectable markers may also be employed.

In embodiments, a plasmid vector can be used to transform a host cell. Plasmid vectors containing replicon and control sequences which are derived from species compatible with the host cell can be used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM.TM. 11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Components of the present invention can be a viral vector that encodes one or more CARs of the invention. Non-limiting examples of virus vectors that may be used to deliver a nucleic acid of the present invention are described herein.

A method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the cells of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

Retroviruses are useful as delivery vectors because of their ability to integrate their genes into the host genome, transferring a large amount of foreign genetic material, infecting a broad spectrum of species and cell types and of being packaged in special cell lines (Miller, 1992).

In order to construct a retroviral vector, a nucleic acid (e.g., one encoding the desired sequence) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al.,

In embodiments, a nucleic acid to be delivered can be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

Suitable methods for nucleic acid delivery for transfection or transformation of cells are known to one of ordinary skill in the art. Such methods include, but are not limited to, direct delivery of DNA such as by ex vivo transfection, by injection, and so forth. Through the application of techniques known in the art, cells may be stably or transiently transformed.

### Ex Vivo Transformation

Methods for transfecting eukaryotic cells and tissues removed from an organism in an ex vivo setting are known to those of skill in the art. Thus, it is contemplated that cells or tissues may be removed and transfected ex vivo using nucleic acids of the present invention. In particular aspects, the transplanted cells or tissues may be placed into an organism. In preferred facets, a nucleic acid is expressed in the transplanted cells.

### Kits of the Invention

Any of the compositions described herein may be comprised in a kit.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In embodiments of the invention, cells that are to be used for cell therapy are provided in a kit, and in some cases the cells are essentially the sole component of the kit. The kit may comprise reagents and materials to make the desired cell. In specific embodiments, the reagents and materials include primers for amplifying desired sequences, nucleotides, suitable buffers or buffer reagents, salt, and so forth, and in some cases the reagents include vectors and/or DNA that encodes a CAR as described herein and/or regulatory elements therefor.

In particular embodiments, there are one or more apparatuses in the kit suitable for extracting one or more samples from an individual. The apparatus may be a syringe, scalpel, and so forth.

In some cases of the invention, the kit, in addition to cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

### Diagnostic Assays

The anti-PD-L1 antibodies can be used diagnostically to, for example, monitor the development or progression of cancer as part of a clinical testing procedure to, e.g., determine the efficacy of a given treatment and/or prevention regimen.

In some aspects, for diagnostic purposes, the anti-PD-L1 antibody of the invention is linked to a detectable moiety, for example, so as to provide a method for detecting a cancer cell in a subject at risk of or suffering from a cancer.

The detectable moieties can be conjugated directly to the antibodies or fragments, or indirectly by using, for example, a fluorescent secondary antibody. Direct conjugation can be accomplished by standard chemical coupling of, for example, a fluorophore to the antibody or antibody fragment, or through genetic engineering. Chimeras, or fusion proteins can be constructed which contain an antibody or antibody fragment coupled to a fluorescent or bioluminescent protein. For example, Casadei, et al, (Proc Natl Acad Sci USA. 1990 Mar; 87(6):2047-51) describe a method of making a vector construct capable of expressing a fusion protein of aequorin and an antibody gene in mammalian cells.

As used herein, the term “labeled”, with regard to the probe or antibody, can encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject (such as a biopsy), as well as tissues, cells and fluids present within a subject. That is, the detection method of the invention can be used to detect cells that express PD-L1 in a biological sample in vitro as well as in vivo. For example, in vitro techniques for detection of PD-L1 include enzyme linked immunosorbent assays (ELISAs), Western blots, immunoprecipitations, and immunofluorescence. Furthermore, in vivo techniques for detection of PD-L1 include introducing into a subject a labeled anti-PD-L1 antibody. For example, the antibody can be labeled with a radioactive marker whose presence and location in a subject can be detected by standard imaging techniques.

In the case of “targeted” conjugates, that is, conjugates which contain a targeting moiety—a molecule or feature designed to localize the conjugate within a subject or animal at a particular site or sites, localization can refer to a state when an equilibrium between bound, “localized”, and unbound, “free” entities within a subject has been essentially achieved. The rate at which such equilibrium is achieved depends upon the route of administration. For example, a conjugate administered by intravenous injection can achieve localization within minutes of injection. On the other hand, a conjugate administered orally can take hours to achieve localization. Alternatively, localization can simply refer to the location of the entity within the subject or animal at selected time periods after the entity is administered. By way of another example, localization is achieved when a moiety becomes distributed following administration.

It is understood that a reasonable estimate of the time to achieve localization can be made by one skilled in the art. Furthermore, the state of localization as a function of time can be followed by imaging the detectable moiety (e.g., a light-emitting conjugate) according to the methods of the invention, such as with a photodetector device. The “photodetector device” used should have a high enough sensitivity to enable the imaging of faint light from within a mammal in a reasonable amount of time, and to use the signal from such a device to construct an image.

In cases where it is possible to use light-generating moieties which are extremely bright, and/or to detect light-generating fusion proteins localized near the surface of the subject or animal being imaged, a pair of “night- vision” goggles or a standard high-sensitivity video camera, such as a Silicon Intensified Tube (SIT) camera (e.g., from Hammamatsu Photonic Systems, Bridgewater, N.J.), can be used. More typically, however, a more sensitive method of light detection is required.

In extremely low light levels the photon flux per unit area becomes so low that the scene being imaged no longer appears continuous. Instead, it is represented by individual photons which are both temporally and spatially distinct form one another. Viewed on a monitor, such an image appears as scintillating points of light, each representing a single detected photon. By accumulating these detected photons in a digital image processor over time, an image can be acquired and constructed. In contrast to conventional cameras where the signal at each image point is assigned an intensity value, in photon counting imaging the amplitude of the signal carries no significance. The objective is to simply detect the presence of a signal (photon) and to count the occurrence of the signal with respect to its position over time.

At least two types of photodetector devices, described below, can detect individual photons and generate a signal which can be analyzed by an image processor. Reduced-Noise Photodetection devices achieve sensitivity by reducing the background noise in the photon detector, as opposed to amplifying the photon signal. Noise is reduced primarily by cooling the detector array. The devices include charge coupled device (CCD) cameras referred to as “backthinned”, cooled CCD cameras. In the more sensitive instruments, the cooling is achieved using, for example, liquid nitrogen, which brings the temperature of the CCD array to approximately −120° C. “Backthinned” refers to an ultra- thin backplate that reduces the path length that a photon follows to be detected, thereby increasing the quantum efficiency. A particularly sensitive backthinned cryogenic CCD camera is the “TECH 512”, a series 200 camera available from Photometries, Ltd. (Tucson, Ariz.).

“Photon amplification devices” amplify photons before they hit the detection screen. This class includes CCD cameras with intensifiers, such as microchannel intensifiers. A microchannel intensifier typically contains a metal array of channels perpendicular to and co-extensive with the detection screen of the camera. The microchannel array is placed between the sample, subject, or animal to be imaged, and the camera. Most of the photons entering the channels of the array contact a side of a channel before exiting. A voltage applied across the array results in the release of many electrons from each photon collision. The electrons from such a collision exit their channel of origin in a “shotgun” pattern, and are detected by the camera.

Even greater sensitivity can be achieved by placing intensifying microchannel arrays in series, so that electrons generated in the first stage in turn result in an amplified signal of electrons at the second stage. Increases in sensitivity, however, are achieved at the expense of spatial resolution, which decreases with each additional stage of amplification. An exemplary microchannel intensifier-based single-photon detection device is the C2400 series, available from Hamamatsu.

Image processors process signals generated by photodetector devices which count photons in order to construct an image which can be, for example, displayed on a monitor or printed on a video printer. Such image processors are typically sold as part of systems which include the sensitive photon-counting cameras described above, and accordingly, are available from the same sources. The image processors are usually connected to a personal computer, such as an IBM-compatible PC or an Apple Macintosh (Apple Computer, Cupertino, Calif.), which may or may not be included as part of a purchased imaging system. Once the images are in the form of digital files, they can be manipulated by a variety of image processing programs (such as “ADOBE PHOTOSHOP”, Adobe Systems, Adobe Systems, Mt. View, Calif.) and printed.

In an embodiment, the biological sample contains protein molecules from the test subject. One preferred biological sample is a peripheral blood leukocyte sample isolated by conventional means from a subject.

The invention also encompasses kits for detecting the presence of PD-L1 or a PD-L1-expressing cell in a biological sample. For example, the kit can comprise: a labeled compound or agent capable of detecting a cancer or tumor cell (e.g., an anti-PD-L1 scFv or monoclonal antibody) in a biological sample; means for determining the amount of PD-L1 in the sample; and means for comparing the amount of PD-L1 in the sample with a standard. The standard is, in some embodiments, a non-cancer cell or cell extract thereof. The compound or agent can be packaged in a suitable container. The kit can further comprise instructions for using the kit to detect cancer in a sample.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Antibody Panning

PD-L1 antibodies of the invention were found via PMPL panning. Briefly, to increase the affinity to PD-L1, the heavy variable regions of anti-PD-L1 antibodies #42 and #50 were cloned into pFarber lambda and kappa light chain display libraries to create #42-light chain shuffling (LCS) library and #52 LCS library, respectively. Each library then panned against PD-L1-mouse Fc soluble antigen for 4 rounds with decreased antigen concentration (3 rounds at lug/ml PD-L1-mFc, 4th round was 0.5 and 0.1 ug/ml respectively; 1 ml to coat the tube). Single colonies were screened against soluble PD-L1-mFc with ELISA. Positive clones were enriched after the 2nd round of panning and the affinity increased. Eluted phage after 3rd round panning will also be cloned into yeast display library to screen for high affinity binders via flow cytometry, if necessary. The anti-PD-L1 #42 & #50 previously discovered from Dr. Marasco's lab have lower affinities to PD-L1 when compared with bench mark commercial antibodies. Knowing that the heavy chains of #42 and #50 contributing mainly to the PD-L1 binding specificity, new single chain Fv phage display libraries were created using the light chain shuffling technique where either #42 or #50 heavy chain variable regions were fused with random kappa and lambda light chain variable regions, resulting #42 LCS and #50 LCS libraries are each with ˜2×10E8 diversity. Panning the new #42 and #52 LCS libraries with decreased antigen concentration lead to discovery of high affinity anti-PD-L1 antibodies containing the original #42 or #50 heavy chain and novel light chain sequences. Other antibodies were discovered via panning with a naive phage library.

### Example 2—Dual Binding Assay

For each well in a 96 well plate, 2E5 CHO-GITR cells were washed with MACS buffer and resuspended in 100 ul MACS buffer. Three fold serial dilutions of bispecific GITR-PDL1 Lc fusion were made in a separate 96 well plate with a starting concentration of 9 ug/ml. Added 50 ul of Ab dilution to 100 ul of buffer with cells, resulting in a final starting concentration of 3 ug/ml. Ab dilutions were as follows: 3, 1, 0.33, 0.111, 0.037, 0.012, 0.004, 0.0014. Cells were incubated with Ab for 30 minute at 4° C., spun down and washed 2× with 250 ul MACS buffer. After final wash, cells were resuspended in MACS buffer with 10 ug/m1 PD-L1-rbFc fusion (extracellular domain of PD-L1) and incubated 4° C. for 30 min. Cells were washed 2× with 250 ul MACS buffer and resuspended in 100 ul MACS buffer with FITC Donkey anti-Rabbit IgG (minimal x-reactivity) antibody (BioLegend cat #406403) at 2 ug/ml. Cells were incubated at 4° C. for 20 minutes. Cells were washed 2× with 250 ul MACS buffer and resuspended in 200 ul MACS buffer. The plate was then read on a Fortessa HTS FACS plate reader.

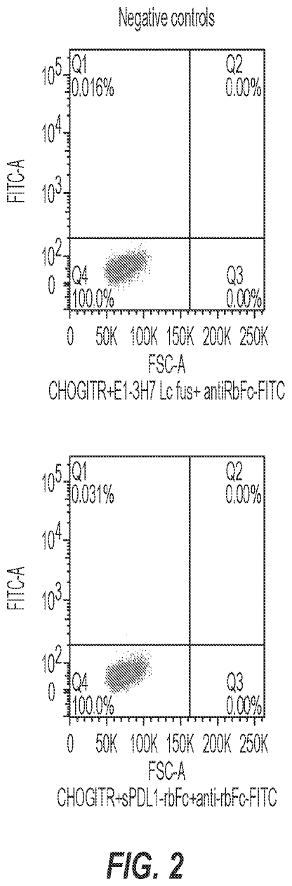

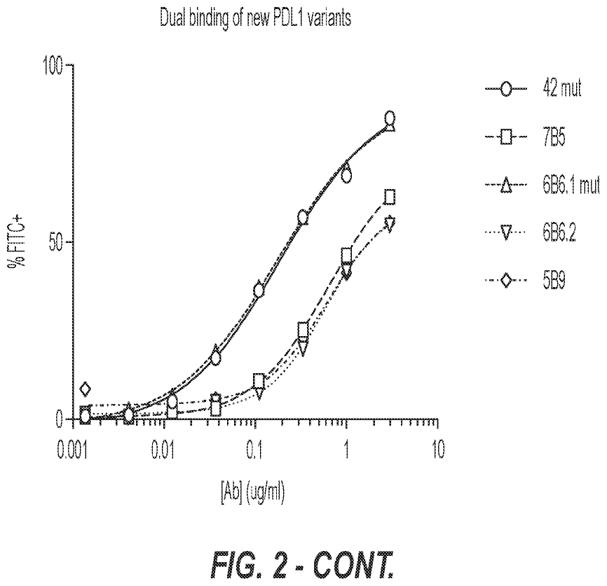

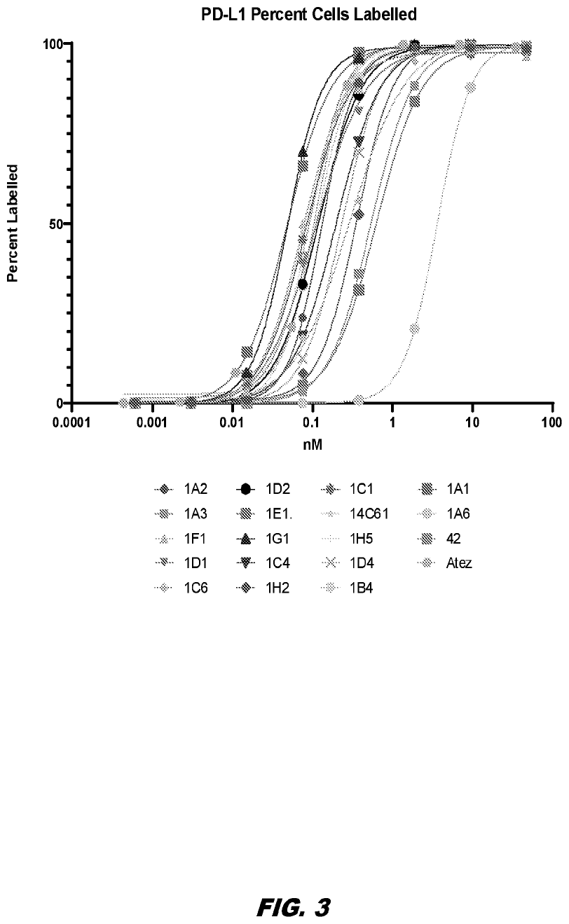

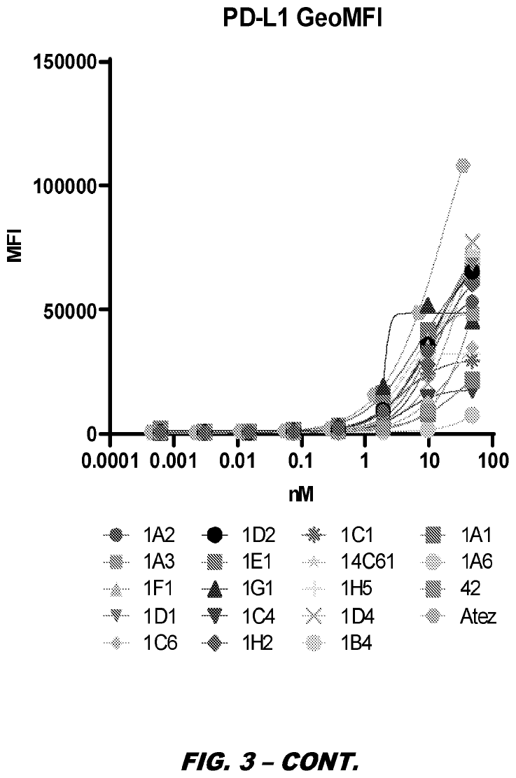

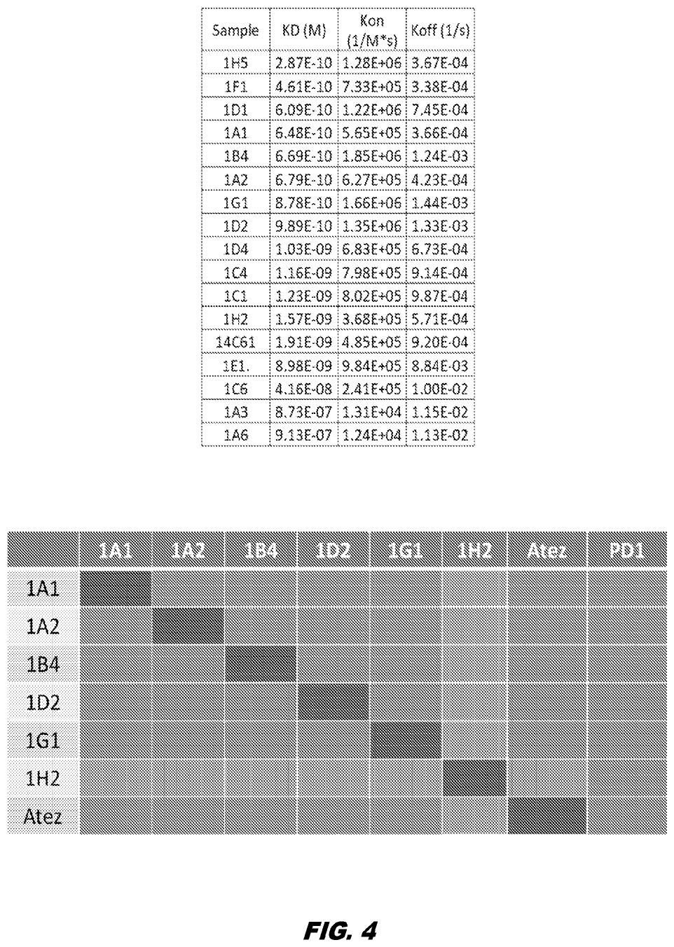

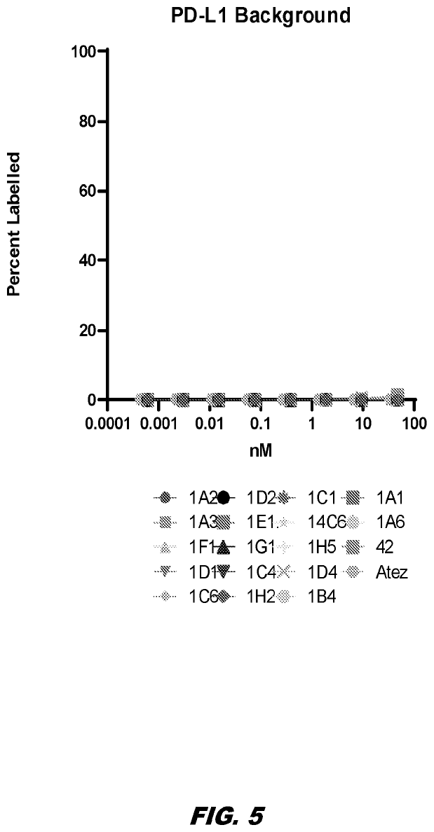

The GITR LC fusion antibodies are able to bind both GITR (membrane bound) and PD-Ll (soluble protein) simultaneously (FIG. 2). The PD-Ll antibodies, 42 mut and 50-6B6.1 mut, are able to bind soluble PD-L1 better than the 50-6B6.2, 50-7B5, and 50-5B9 antibodies as a Light Chain Fusion.

### Example 3—Mixed lymphocyte reaction (MLR) Protocol

CD14+monocytes were isolated using Miltenyi CD14+ microbeads. The cells were cultured in Miltenyi Mo-DC media (pre-prepared media with GM-CSF +IL4). The cells were cultured for 5 days, then the following was added: TNF-α (1000 U/ml), IL-β (5 ng/ml), IL-6 (10 ng/ml) and prostaglandin E2 (PGE2) (1 μM) and the cells were cultured for 2 days to mature DC. T cells were isolated the day of the MLR experiment (CD4+ negative selection kit StemCell). 100,000 T cells and 10,000 MoDC cells were used per well for MLR. Antibodies were added at various concentrations and the cultures were incubated for 5 days.

Supernatant was saved for ELISA screening (e.g., IFNγ).

MLR atezolizumab vs anti PDL1 abs. 1 T cell donors and 1 DC donors were used.

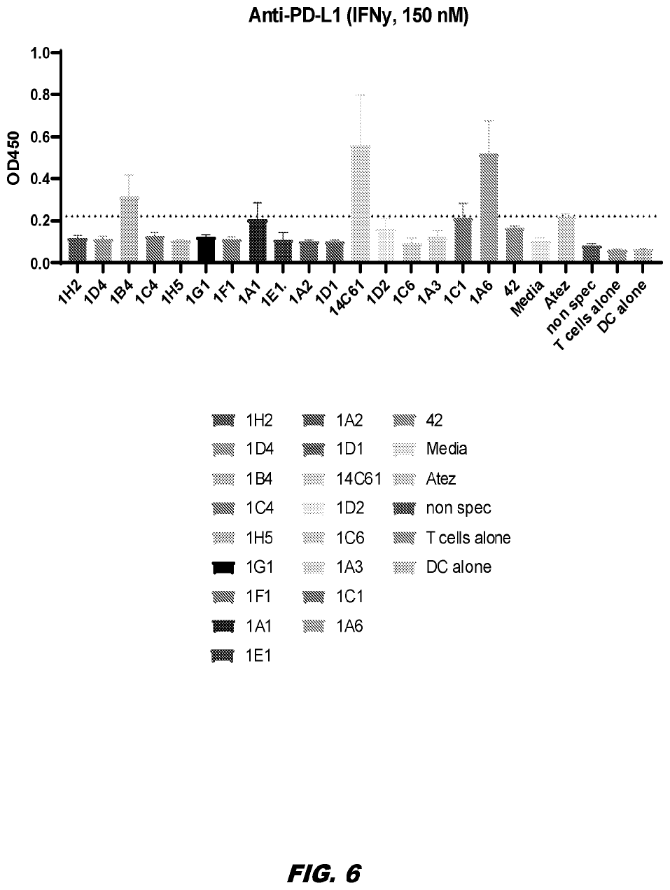

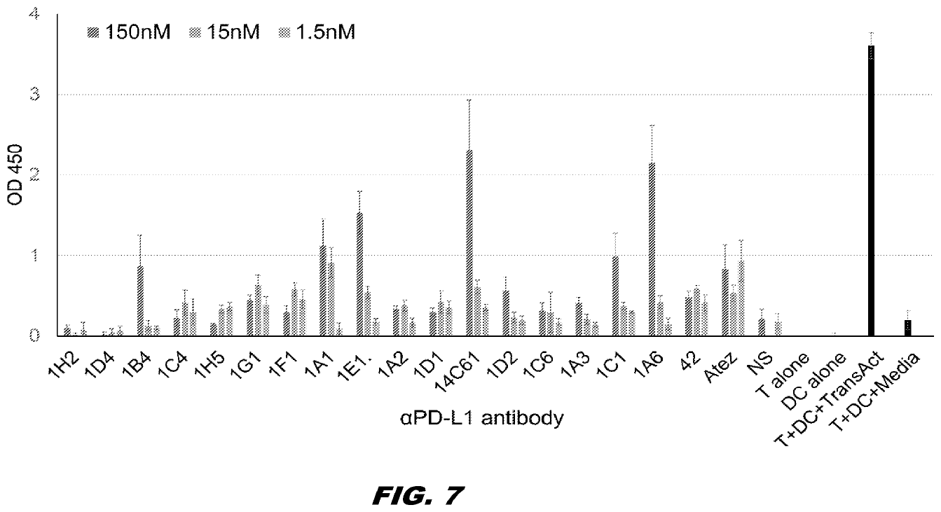

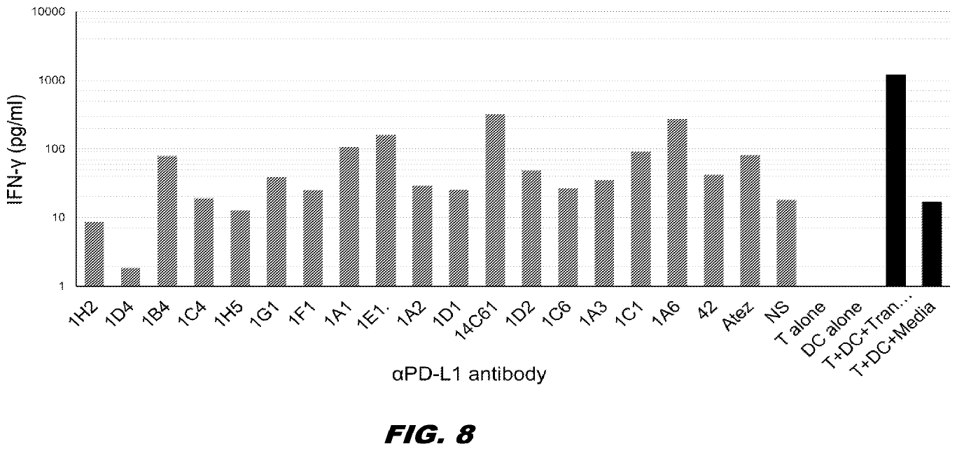

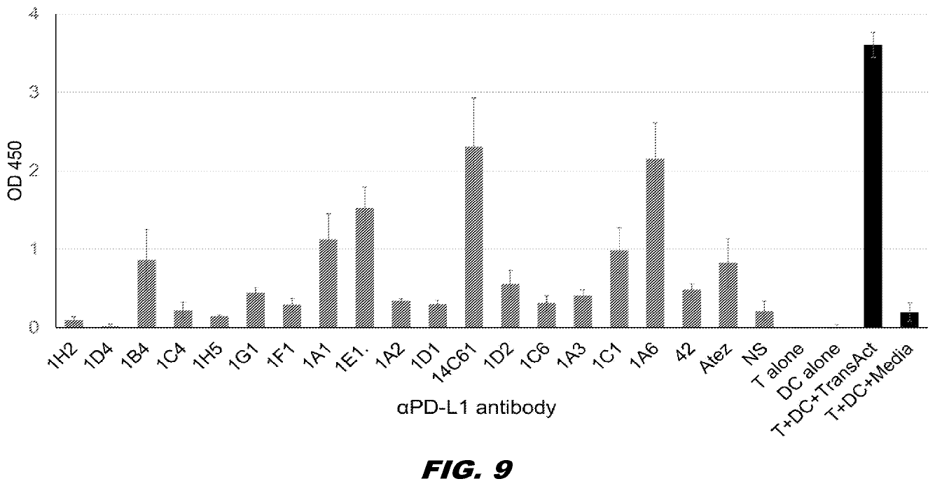

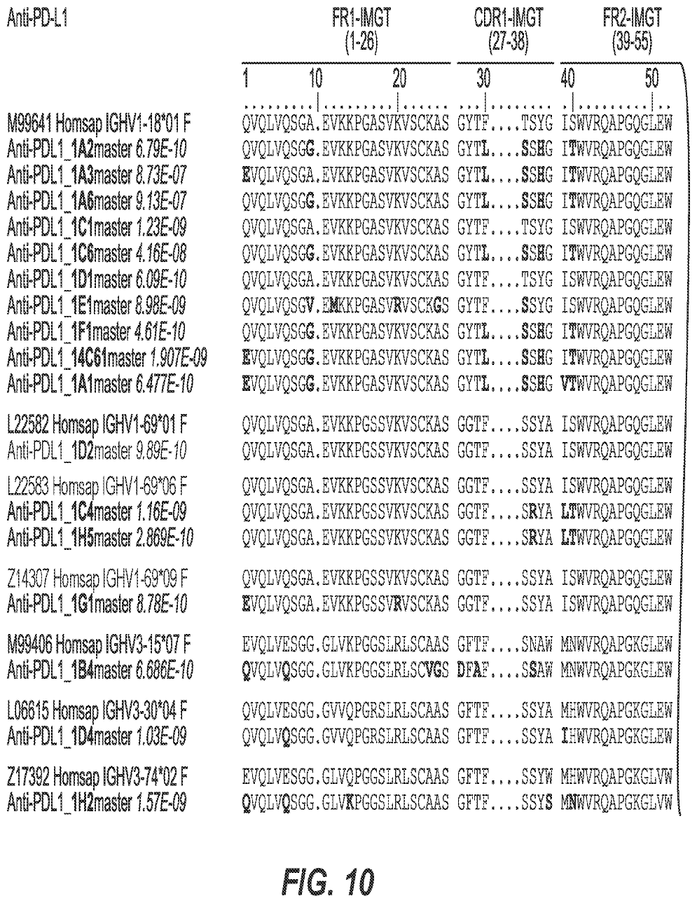

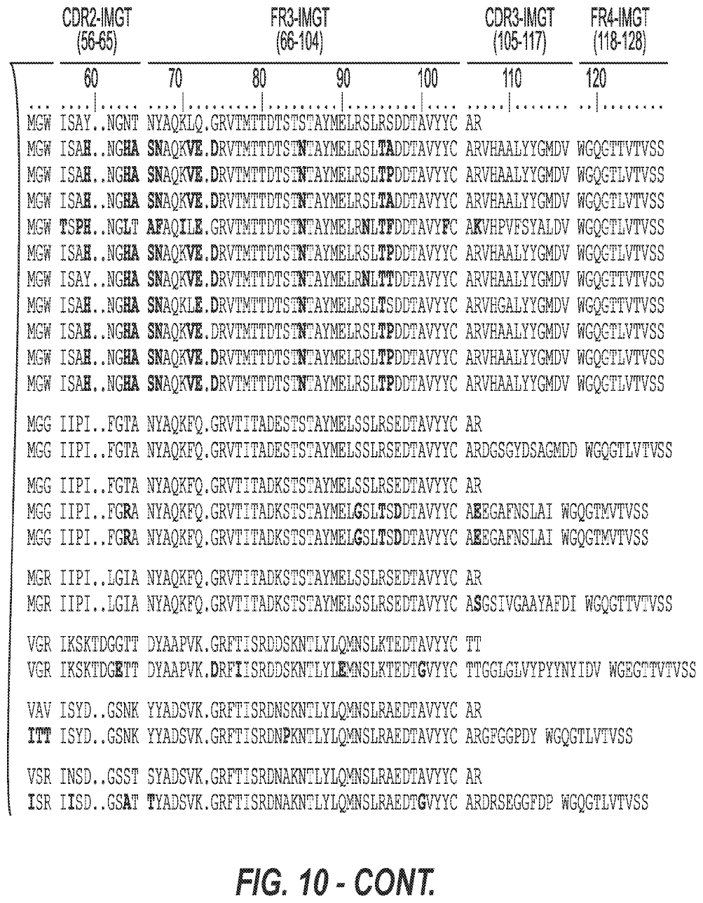

The anti-PDL1 abs in scFv-Fc format was tested against a commercial preparation of atezolizumab, anti-PDL1 #42 scFv-Fc and a nonspecific Ab control. As shown in FIGS. 6-9, addition of certain anti-PDL1 abs including atezolizumab and anti-PDL1 #42 lead to an increase in cytokine production compared to that of the nonspecific control.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1.-64. (canceled)

65. An isolated antibody or fragment thereof that binds to human PD-L1 protein and comprises a heavy chain variable region comprising an amino acid sequence selected from the group consisting of SEQ ID NO: 16, 52, 54, 56, 60, 62, 64, 66, 68, 70, 72, 74, 76, 78, 80, and 82, and
a light chain variable region comprising an amino acid sequence selected from the group consisting of SEQ ID NOS: 31, 38, 42, 46, 53, 55, 57, 59, 61, 63, 65, 67, 69, 71, 73, 75, 77, 79, 81, and 83.

66. An isolated antibody or fragment thereof that binds to human PD-L1 protein and comprises a heavy chain variable region comprising:
a VH-CDR1 having an amino acid sequence selected from the group consisting of SEQ ID NOS: 2, 10, 84, 85, 86, 87, 88, 89, and 90,
a VH-CDR2 having an amino acid sequence selected from the group consisting of SEQ ID NOS: 4, 12, 91, 92, 93, 94, 95, 96, 97, and 98, and/or
a VH-CDR3 having an amino acid sequence selected from the group consisting of SEQ ID NOS: 14, 99, 100, 101, 102, 103, 104, 105, 106, and 107; and/or
a light chain variable region comprising:
a VL-CDR1 having an amino acid sequence selected from the group consisting of SEQ ID NOS: 26, 33, 40, 44, 108, 109, 110, 111, 112, 113, 114, 115, 116, and 117,
a VL -CDR2 having an amino acid sequence selected from the group consisting of SEQ ID NOS: 20, 28, 35, 45, 118, 119, 120, 121, 122, 123, 124, and 125, and/or
a VL-CDR3 having an amino acid sequence selected from the group consisting of SEQ ID NOS: 30, 37, 126, 127, 128, 129, 130, 131, 132, 133, 134, and 135.

67. An isolated antibody or fragment thereof that binds to human PD-L1 protein and wherein the antibody comprises:
a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGSKG (SEQ ID NO:26), DDR (SEQ ID NO:28), and QVWDSGSDHWV (SEQ ID NO:30) respectively;
a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGDKG (SEQ ID NO:33), DDS (SEQ ID NO:35), and QVWDSSSDHWV (SEQ ID NO:37) respectively;
a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGNKG (SEQ ID NO:40), DDS (SEQ ID NO:35), and QVWDSSSDHWV (SEQ ID NO:37) respectively;
a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGGKG (SEQ ID NO:44), DDY (SEQ ID NO:45), and QVWDSSSDHWV (SEQ ID NO:37) respectively;
a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIESRS (SEQ ID NO: 108, DDT (SEQ ID NO: 118), and QVWDSSGDLWV (SEQ ID NO: 126) respectively;
a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGSKG (SEQ ID NO: 26, DDS (SEQ ID NO:35), and QVWDSSSDHWV (SEQ ID NO:37) respectively;
a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGSKS (SEQ ID NO: 109, DDS (SEQ ID NO:35), and QVWDSSSDHWV (SEQ ID NO:37);
a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGSKG (SEQ ID NO: 26, DDS (SEQ ID NO:35), and QVWDSSSDHWV (SEQ ID NO:37) respectively;
a heavy chain variable region with three CDRs comprising the amino acid sequences DFAFSSAW (SEQ ID NO: 84), IKSKTDGETT (SEQ ID NO: 91), and TTGGLGLVYPYYNYIDV (SEQ ID NO: 99) respectively, and/or a light chain variable region with three CDRs comprising the amino acid sequences SSNIGSNY (SEQ ID NO: 110, RNN (SEQ ID NO: 119), and AAWDDSLNGLV (SEQ ID NO: 127) respectively;
a heavy chain variable region with three CDRs comprising the amino acid sequences GYTFTSYG (SEQ ID NO: 85), TSPHNGLT (SEQ ID NO: 92), and AKVHPVFSYALDV (SEQ ID NO: 100) respectively, and/or a light chain variable region with three CDRs comprising the amino acid sequences SGSIASNY (SEQ ID NO: 111, EDN (SEQ ID NO: 20), and QSYDGITVI (SEQ ID NO: 128) respectively;
a heavy chain variable region with three CDRs comprising the amino acid sequences GGTFSRYA (SEQ ID NO: 86), IIPIFGRA (SEQ ID NO: 93), and AEEGAFNSLAI (SEQ ID NO: 101) respectively, and/or a light chain variable region with three CDRs comprising the amino acid sequences SGSIASNY (SEQ ID NO: 111), ADN (SEQ ID NO: 120), and QSYDSSNHWV (SEQ ID NO: 129) respectively;
a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGSKS (SEQ ID NO: 109, DDS (SEQ ID NO:35), and QVWDSSSDHWV (SEQ ID NO:37) respectively;
a heavy chain variable region with three CDRs comprising the amino acid sequences GYTFTSYG (SEQ ID NO: 85), ISAYNGHA (SEQ ID NO: 94), and ARVHAALYYGMDV (SEQ ID NO: 14) respectively, and/or a light chain variable region with three CDRs comprising the amino acid sequences NIGSKG (SEQ ID NO: 26, DDS (SEQ ID NO: 35), and QVWDSRSDHWV (SEQ ID NO: 130) respectively;
a heavy chain variable region with three CDRs comprising the amino acid sequences GGTFSSYA (SEQ ID NO: 87), IIPIFGTA (SEQ ID NO: 95), and ARDGSGYDSAGMDD (SEQ ID NO: 102) respectively, and/or a light chain variable region with three CDRs comprising the amino acid sequences RSNIGSNY (SEQ ID NO: 112, SNN (SEQ ID NO: 121), and AVWDDSLSGVV (SEQ ID NO: 131) respectively;
a heavy chain variable region with three CDRs comprising the amino acid sequences GFTFSSYA (SEQ ID NO: 88), ISYDGSNK (SEQ ID NO: 96), and ARGFGGPDY (SEQ ID NO: 103) respectively, and/or a light chain variable region with three CDRs comprising the amino acid sequences SGINVGTYR (SEQ ID NO: 113), YKSDSDK (SEQ ID NO: 122), and MIWHSSAYV (SEQ ID NO: 132) respectively;
a heavy chain variable region with three CDRs comprising the amino acid sequences GYTFSSYG (SEQ ID NO: 89), ISAHNGHA (SEQ ID NO: 12), and ARVHGALYYGMDV (SEQ ID NO: 104) respectively, and/or a light chain variable region with three CDRs comprising the amino acid sequences NIGGKS (SEQ ID NO: 114, DDR (SEQ ID NO: 28), and QVWDSSSDHWV (SEQ ID NO: 37) respectively;
a heavy chain variable region with three CDRs comprising the amino acid sequences GYTLSSHG (SEQ ID NO: 10), ISAHNGHA (SEQ ID NO: 12), and ARVHAALYYGMDV (SEQ ID NO: 14) respectively, and/or a light chain variable region with three CDRs comprising the amino acid sequences NIGSKG (SEQ ID NO: 26, DDR (SEQ ID NO: 28), and QVWDSSSDHWV (SEQ ID NO: 37) respectively;
a heavy chain variable region with three CDRs comprising the amino acid sequences GGTFSSYA (SEQ ID NO: 87), IlPILGIA (SEQ ID NO: 97), and ASGSIVGAAYAFDI (SEQ ID NO: 105) respectively, and/or a light chain variable region with three CDRs comprising the amino acid sequences NIGGRV (SEQ ID NO: 115), DDT (SEQ ID NO: 123, and QVWDSRSDHPV (SEQ ID NO: 133) respectively;
a heavy chain variable region with three CDRs comprising the amino acid sequences GFTFSSYS (SEQ ID NO: 90), IISDGSAT (SEQ ID NO: 98), and ARDRSEGGFDP (SEQ ID NO: 106) respectively, and/or a light chain variable region with three CDRs comprising the amino acid sequences SLRSYY (SEQ ID NO: 116), GKN (SEQ ID NO: 124, and NSRDISDNQWQWI (SEQ ID NO: 134) respectively; or
a heavy chain variable region with three CDRs comprising the amino acid sequences GGTFSRYA (SEQ ID NO: 86), IIPIFGRA (SEQ ID NO: 93), and AEEGAFNSLAI (SEQ ID NO: 107) respectively, and/or a light chain variable region with three CDRs comprising the amino acid sequences SGSIASHF (SEQ ID NO: 117), GDD (SEQ ID NO: 125), and QSYDSSNHVV (SEQ ID NO: 135) respectively.

68.-72. (canceled)

73. An isolated monoclonal antibody or antigen-binding fragment thereof that binds to PD-L1 comprising a heavy chain, a light chain, or a combination thereof, wherein
the heavy chain comprises an amino acid sequence about 95% identical to SEQ ID NO: 16, and the light chain comprises an amino acid sequence about 95% identical to SEQ ID NO: 31;
the heavy chain comprises an amino acid sequence about 95% identical to SEQ ID NO: 16, and the light chain comprises an amino acid sequence about 95% identical to SEQ ID NO: 38;
the heavy chain comprises an amino acid sequence about 95% identical to SEQ ID NO: 16, and the light chain comprises an amino acid sequence about 95% identical to SEQ ID NO: 42;
the heavy chain comprises an amino acid sequence about 95% identical to SEQ ID NO: 16, and the light chain comprises an amino acid sequence about 95% identical to SEQ ID NO: 46;
the heavy chain comprises an amino acid sequence about 95% identical to SEQ ID NO: 52, and the light chain comprises an amino acid sequence about 95% identical to SEQ ID NO: 53;
the heavy chain comprises an amino acid sequence about 95% identical to SEQ ID NO: 54, and the light chain comprises an amino acid sequence about 95% identical to SEQ ID NO: 55;
the heavy chain comprises an amino acid sequence about 95% identical to SEQ ID NO: 56, and the light chain comprises an amino acid sequence about 95% identical to SEQ ID NO: 57;
the heavy chain comprises an amino acid sequence about 95% identical to SEQ ID NO: 16, and the light chain comprises an amino acid sequence about 95% identical to SEQ ID NO: 59;
the heavy chain comprises an amino acid sequence about 95% identical to SEQ ID NO: 60, and the light chain comprises an amino acid sequence about 95% identical to SEQ ID NO: 61;
the heavy chain comprises an amino acid sequence about 95% identical to SEQ ID NO: 62, and the light chain comprises an amino acid sequence about 95% identical to SEQ ID NO: 63;
the heavy chain comprises an amino acid sequence about 95% identical to SEQ ID NO: 64, and the light chain comprises an amino acid sequence about 95% identical to SEQ ID NO: 65;
the heavy chain comprises an amino acid sequence about 95% identical to SEQ ID NO: 66, and the light chain comprises an amino acid sequence about 95% identical to SEQ ID NO: 67;
the heavy chain comprises an amino acid sequence about 95% identical to SEQ ID NO: 68, and the light chain comprises an amino acid sequence about 95% identical to SEQ ID NO: 69;
the heavy chain comprises an amino acid sequence about 95% identical to SEQ ID NO: 70, and the light chain comprises an amino acid sequence about 95% identical to SEQ ID NO: 71;
the heavy chain comprises an amino acid sequence about 95% identical to SEQ ID NO: 72, and the light chain comprises an amino acid sequence about 95% identical to SEQ ID NO: 73;
the heavy chain comprises an amino acid sequence about 95% identical to SEQ ID NO: 74, and the light chain comprises an amino acid sequence about 95% identical to SEQ ID NO: 75;
the heavy chain comprises an amino acid sequence about 95% identical to SEQ ID NO: 76, and the light chain comprises an amino acid sequence about 95% identical to SEQ ID NO: 77;
the heavy chain comprises an amino acid sequence about 95% identical to SEQ ID NO: 78, and the light chain comprises an amino acid sequence about 95% identical to SEQ ID NO: 79;
the heavy chain comprises an amino acid sequence about 95% identical to SEQ ID NO: 80, and the light chain comprises an amino acid sequence about 95% identical to SEQ ID NO: 81; or
the heavy chain comprises an amino acid sequence about 95% identical to SEQ ID NO: 82; and the light chain comprises an amino acid sequence about 95% identical to SEQ ID NO: 83.

74. (canceled)

75. An isolated bispecific antibody comprising a fragment of claim 67, and a second antigen-binding fragment having specificity to a molecule on an immune cell.

76. The bispecific antibody of claim 75, wherein the molecule is selected from the group consisting of B7H3, B7H4, CD27, CD28, CD40, CD40L, CD47, CD122, CTLA-4, GITR, GITRL, ICOS, ICOSL, LAG-3, LIGHT, OX-40, OX40L, PD-1, TIM3, 4-1BB, TIGIT, VISTA, HEVM, BTLA, and KIR.

77. The bispecific antibody of claim 75, wherein the fragment and the second fragment each is independently selected from a Fab fragment, a single-chain variable fragment (scFv), or a single-domain antibody.

78. The bispecific antibody of claim 75, further comprising a Fc fragment.

79. A nucleic acid encoding the antibody according to claim 67.

80. A nucleic acid encoding the bispecific antibody according to claim 75.

81. A pharmaceutical composition comprising the antibody or fragment thereof according to claim 67, and a pharmaceutically acceptable carrier or excipient.

82. The pharmaceutical composition of claim 81, further comprising at least one additional therapeutic agent.

83. The pharmaceutical composition of claim 82, wherein the therapeutic agent is a toxin, a radiolabel, a siRNA, a small molecule, or a cytokine.

84. A pharmaceutical composition comprising the bispecific antibody according to claim 75, and a pharmaceutically acceptable carrier or excipient.

85. The pharmaceutical composition of claim 84, further comprising at least one additional therapeutic agent.

86. The pharmaceutical composition of claim 85, wherein the therapeutic agent is a toxin, a radiolabel, a siRNA, a small molecule, or a cytokine.

87. An isolated cell comprising one or more polynucleotide(s) encoding the antibody or fragment thereof according to claim 67.

88. An isolated cell comprising one or more polynucleotide(s) encoding the bispecific antibody or fragment thereof according to claim 75.

89. A vector comprising the nucleic acid of claim 79.

90. A cell comprising the vector of claim 89.

91. A kit comprising: the at least one antibody according to claim 67; a syringe, needle, or applicator for administration of the at least one antibody to a subject; and instructions for use.

92. A method of treating cancer in a subject, the method comprising administering to a subject in need thereof a therapeutically effective amount of a composition comprising an antibody according to claim 67.

93. The method of claim 92, further comprising administering to the subject a chemotherapeutic agent.

94. The method of claim 92, wherein the cancer is a checkpoint blockade cancer.

95. A chimeric antigen receptor (CAR) comprising an intracellular signaling domain, a transmembrane domain and an extracellular domain, wherein the extracellular domain is an isolated monoclonal antibody or antigen-binding fragment thereof that binds to human Programmed death-ligand 1 (PD-L1) protein, wherein the monoclonal antibody or fragment thereof comprises an antibody according to claim 67.

96. The CAR of claim 95, wherein the transmembrane domain further comprises a stalk region positioned between the extracellular domain and the transmembrane domain.

97. The CAR or claim 95, wherein the transmembrane domain comprises CD28.

98. The CAR of claim 95, further comprising one or more additional costimulatory molecules positioned between the transmembrane domain and the intracellular signaling domain

99. The CAR of claim 98, wherein the costimulatory molecules is CD28, 4-1BB, ICOS, or OX40.

100. The CAR of claim 95, wherein the intracellular signaling domain comprises a CD3 zeta chain.

101. The CAR of claim 95, wherein the antibody is a Fab or a scFV.

102. A nucleic acid encoding the CAR according to claim 95.

103. The nucleic acid of claim 102, further comprising a nucleic acid encoding a polypeptide positioned after the intracellular signaling domain.

104. The nucleic acid of claim 103, wherein the polypeptide is an antibody a cytokine.

105. The nucleic acid of claim 104, wherein the antibody is a scFV.

106. (canceled)

107. A vector comprising the nucleic acid according to claim 102.

108. A cell comprising the vector of claim 107.

109. A genetically engineered cell which expresses and bears on the cell surface membrane the chimeric antigen receptor according to claim 95.

110. The genetically engineered cell of claim 109, wherein the cell is a T-cell or an NK cell.

111. The genetically engineered cell of claim 110, wherein the T cell is CD4⁺ or CD8⁺.

112. The genetically engineered cell of claim 111, which comprises a mixed population of CD4⁺ and CD8 cells⁺.

113. (canceled)

114. A vector comprising the nucleic acid of claim 80.

115. A cell comprising the vector of claim 114.
